SEQUENCE LISTING (64 sequences)

SEQ ID NO: 1 - DNA, 1131 bp, Oryza sativa
atgctgacggataggtggggagggaggagggaggcgatggagttcgggaacggcgggtcctcctcgtcggagcgcagggcggcggcggagggggcgacgctggcgagacagggttcggtgtattcgctgacgttcgacgagttccagagcgcgctcgctggtggcggcggcggcggcggtggtggaagcgggttcgggaaggacttcggctccatgaacatggacgagctgctccggagcatctggaccgcggaggagagccaggccatggcctccgcctctggctccgccgcgggggtgggcgtggccgtgggggcgccgccgacgtcgctgcagcgccagggctcgctcaccctgccccgcacgctcagcgccaagacggtggacgaggtatggcgcaaccttgtgcgcgacgagccaccgccggtgggggcggcggatggcggcgatatgccaccccagcggcagtcgaccctcggggagatgaccctggaggagttcttggtcagagccggcgtggtccgagaaaatccacccgctgcgccgccccccgttcccccgccaatgccgccgcggccagtcccggtggtccccaagaccactgccttcttgggaaacttccccggtgccaacgatgccggcgcggcggcgctgggctttgcgccgctcggcatgggggatccagccttgggcaatggcctgatgcccagggcagtgccagtgggcttgcctggcgccgccgtcgctatgcaaacagcggtgaaccagtttgattctggcgataaggggaacagcgacctgtcatcgccgacggagccaatgccttattccttcgaagggttggtgagggggagaaggaacggtggcggagtagagaaagtggtggagaggaggcagaggaggatgatcaagaacagggagtccgcggcgagatcccgcgcgcgcaagcaggcttacacattggaattggaagctgaagttcagaaactcaaggagatgaacaaggaattggagaggaaacaggcagatatcatggaaatgcagaaaaatgaggtagaagaaatgataaaggatccatttggaagaaggaagagactttgcttgcgaagaacactgactggtccctggtga

SEQ ID NO: 2 - protein, 376 aa, Oryza sativa
MLTDRWGGRREAMEFGNGGSSSSERRAAAEGATLARQGSVYSLTFDEFQSALAGGGGGGGGGSGFGKDFGSMNMDELLRSIWTAEESQAMASASGSAAGVGVAVGAPPTSLQRQGSLTLPRTLSAKTVDEVWRNLVRDEPPPVGAADGGDMPPQRQSTLGEMTLEEFLVRAGVVRENPPAAPPPVPPPMPPRPVPVVPKTTAFLGNFPGANDAGAAALGFAPLGMGDPALGNGLMPRAVPVGLPGAAVAMQTAVNQFDSGDKGNSDLSSPTEPMPYSFEGLVRGRRNGGGVEKVVERRQRRMIKNRESAARSRARKQAYTLELEAEVQKLKEMNKELERKQADIMEMQKNEVEEMIKDPFGRRKRLCLRRTLTGPW

SEQ ID NO: 3 - DNA, 1113 bp, Oryza sativa
atggagaaatacgagccagttcgggagatcggggcgggcaacttcggggtagcgaagctgatgcggaacaaggagacgcgggagctggtggcgatgaagttcatcgagagagggaacaggatcgacgagaacgtgttccgggagatcgtgaatcatcgttcgctgcgtcacccgaacataataaggttcaaggaggtggtggtgacggggaggcatctggcgatcgtgatggagtacgcggcgggaggggagctgttcgagaggatatgcgaggcggggaggttccacgaggacgaggcgcgctacttcttccagcagctggtgtgcggggtgagctactgccacgccatgcagatctgccaccgcgacctcaagctggagaatacgctgctggacggcagcccggccccgcgcctcaagatctgcgacttcggctactccaagtcctccctcctccactcccgccccaaatccaccgtcggcacccccgcctacatcgcccccgaggtcctctcccgccgcgagtacgacggcaagctcgccgacgtctggtcctgcggcgtcaccctctacgtcatgctcgtcggcgcttaccctttcgaggatcccaaggaccccaagaacttcagaaagaccatctcgcgcatcatgtccgtccagtacaagatccccgagtacgtccacgtctcccagccctgccgccacctcctctcccgcatcttcgtcgccaacccctacaagcgcatcagcatgggcgagatcaagagccacccctggttcctcaagaacctgccgcgcgagctcaaggaggaggcgcaggccgtctactacaaccgccggggagccgatcacgcggcttccagcgcaagtagtgcggctgctgcagctgccttctcgccgcagagcgtggaggacatcatgaggatcgtgcaggaggcgcagaccgtccccaagcccgacaagcccgtctctggctacggctggggcaccgacgacgacgacgacgaccaacaaccagctgaggaggaggacgaagaagacgactacgacaggacggtgcgcgaggttcacgccagcgtcgacctcgacatgtcaaacctccaaatctcctga

SEQ ID NO: 4 - protein, 370 aa, Oryza sativa
MEKYEPVREIGAGNFGVAKLMRNKETRELVAMKFIERGNRIDENVFREIVNHRSLRHPNIIRFKEVVVTGRHLAIVMEYAAGGELFERICEAGRFHEDEARYFFQQLVCGVSYCHAMQICHRDLKLENTLLDGSPAPRLKICDFGYSKSSLLHSRPKSTVGTPAYIAPEVLSRREYDGKLADVWSCGVTLYVMLVGAYPFEDPKDPKNFRKTISRIMSVQYKIPEYVHVSQPCRHLLSRIFVANPYKRISMGEIKSHPWFLKNLPRELKEEAQAVYYNRRGADHAASSASSAAAAAAFSPQSVEDIMRIVQEAQTVPKPDKPVSGYGWGTDDDDDDQQPAEEEDEEDDYDRTVREVHASVDLDMSNLQIS

SEQ ID NO: 5 - DNA, 1386 bp, Oryza sativa
atgccacacagagcaggagaggtgtcgtcgtcgtcttctccggggagcatgtcgaaggcgatgcagtgcttcgggttcgccggctgggagagggatgagcggcgtggccggtcgtcggcggtggcctcggcggcggcggcgacgacgcgttcgctgtcggcgcggtccaacagcagcacgtcgacggaccgcgacgcgcggcggtcggggtcggagtgctccctgaacgtgtcgtcggagatcagcgccgagtcgttcggccggtaccggcagctgtcgctgccgcagcgcgcgagcaacaacctccgcatcttcaccttccaggagctcaagagcgccacccgcgggttcagccgctcgctggtgctcggcgagggcggcttcggctgcgtctaccgcggcaccatccgcagcgtcctcgagcctcgccggagcgtcgaggtcgccatcaagcagctcggccgcaaaggcctacaggggcataaggagtgggtgacggaggtgaacgttcttggggtggtggatcatccgaacctggtgaagctcatcgggtactgcgccgaggacgacgagagggggatgcagctgctgctcgtctacgagttcatgcccaacgggagcctggcagatcacctgtcgtcgaggtcgccgaggccggcgtcgtgggcgatgcggctcagggtggcgctcgacaccgcgcgcggcttgaagtatctccatgaagaatctgaaatcaagataatatttcgtgatctgaagccttccaacattctgatcgacgagaactggaacgcgaagctgtcagacttcggattggctagactggtatctcaggatggcagccatgtctccactgcggtggtgggaactattgggtatgcagctccagagtacatccacacagggcgcctcagcagcaagaacgacatatggagctacggcgtggtgctctacgaactccttaccggtcggcgacctctggacaggaacaggccgagaggcgaacagaacctcatagagtgggtgaagccctactccactgactcaaagaagcttgaaatcataatggatccaaggcttgaagggagttacagcttgaagtcggcggccaagctcgcctcggtggcaaacaagtgcctcgtgcgccacgccaggcaccggccaaagatgagcgaggtgctggagatggtgcagaagatcgtcgatagcactgacctcggaacgccggagcatcccctcatcagcaagtcaagagagctgacgcgtgatgagaagaaacgaaaagggcttgacctgaagagaagatttgctgatattaaagctggaggggatcagagatggtttacatggcagagatggagacccaaacttgtcagaacacaatga

SEQ ID NO: 6 - protein, 461 aa, Oryza sativa
MPHRAGEVSSSSSPGSMSKAMQCFGFAGWERDERRGRSSAVASAAAATTRSLSARSNSSTSTDRDARRSGSECSLNVSSEISAESFGRYRQLSLPQRASNNLRIFTFQELKSATRGFSRSLVLGEGGFGCVYRGTIRSVLEPRRSVEVAIKQLGRKGLQGHKEWVTEVNVLGVVDHPNLVKLIGYCAEDDERGMQLLLVYEFMPNGSLADHLSSRSPRPASWAMRLRVALDTARGLKYLHEESEIKIIFRDLKPSNILIDENWNAKLSDFGLARLVSQDGSHVSTAVVGTIGYAAPEYIHTGRLSSKNDIWSYGVVLYELLTGRRPLDRNRPRGEQNLIEWVKPYSTDSKKLEIIMDPRLEGSYSLKSAAKLASVANKCLVRHARHRPKMSEVLEMVQKIVDSTDLGTPEHPLISKSRELTRDEKKRKGLDLKRRFADIKAGGDQRWFTWQRWRPKLVRTQ

SEQ ID NO: 7 - DNA, 1242 bp, Oryza sativa
atggcccggctgctactctccggcgtcgcgccgctccccctcctcccctgccgccgtcgcgccattgcatttgcgctcccacttgggaatgtccgcctccgcctccgtgtggccgcacccaccagccgcgtggccaccgtggaggaggacgacaatgaaaataatgctcctcctcctccttgtgaggacttcgacccgggcgcggcgcccccgtttggtctggccgacatccgcgccgctatccccaagcactgttgggtgaaggacccctggcgatccatggggtacgtgctgcgcgacgtggtggtggtgttcgccctcgctgccgccgccgcgcgcctccacagctgcctcgcctggccgctctactgggcggcgcagggaaccatgttctgggcgctcttcgtcctcggccacgactgcgggcacgggagcttctccaacaactcgaggctcaacagcgtgatgggccacatactccactcctccatcctcgtaccctaccatggctggaggattagccacaggacgcatcatcagaaccatggccatgtcgacaaggatgagtcctggcatcccctccccgagcggctgtacaggagccttaacagagccacccggatgctccgcttctccatacccttccccatgctcgcctacccattctacctgtggtctcggagcccaggaaagtctggttcgcatttccatcccagcagcgacctgttccagcccaacgaaaggaacgatgtgctgacatccacagcgtgctgggtggccatggctgccctcctcgcaggcctcaccttcctcatgggacccctcctcatgctcaacctctactttgtcccttactggatttttgttatgtggctggacttcgtcacctacttgcaccatcacggccacaacgacaagctgccctggtaccgtggcaaggaatggagctatttgcggggaggactgacgacagtggacagggactatgggtggatcaacaacatccaccacgacatcgggacacatgtcattcaccatcttttcccccaaatcccgcattaccatctaattgaggcgacggaagcagcaaagggtgtgatggggaaatactacagggagccggacaagtctgggccttttcccttacacctgtttggagcgctgtcccggagcttgaaacgcgaccactatgtcagcgacaccggagatgtggtctactaccagaccgaccctgctaactaa

SEQ ID NO: 8 - protein, 413 aa, Oryza sativa
MARLLLSGVAPLPLLPCRRRAIAFALPLGNVRLRLRVAAPTSRVATVEEDDNENNAPPPPCEDFDPGAAPPFGLADIRAAIPKHCWVKDPWRSMGYVLRDVVVVFALAAAAARLHSCLAWPLYWAAQGTMFWALFVLGHDCGHGSFSNNSRLNSVMGHILHSSILVPYHGWRISHRTHHQNHGHVDKDESWHPLPERLYRSLNRATRMLRFSIPFPMLAYPFYLWSRSPGKSGSHFHPSSDLFQPNERNDVLTSTACWVAMAALLAGLTFLMGPLLMLNLYFVPYWIFVMWLDFVTYLHHHGHNDKLPWYRGKEWSYLRGGLTTVDRDYGWINNIHHDIGTHVIHHLFPQIPHYHLIEATEAAKGVMGKYYREPDKSGPFPLHLFGALSRSLKRDHYVSDTGDVVYYQTDPAN

SEQ ID NO: 9 - DNA, 1077 bp, Oryza sativa
atgtcgcaggccgccgggaacccctacgccgctgagctcgccgccgccaagaaggccgtcaccctcgccgcccgcctctgccaggcggtgcaaaaggacattctgcagtctggtgttcagtctaaggcggatcaaagtccggtgacagttgccgattatgggtctcaaatattggtaagccttgtcttgaaaatggaagcaccagcttcttcttccttctctatggtggctgaggaggactcggaagaattgaggaaagaaggcgcagaagaaattttagaaaatatcaccgagctcgtaaacgaaactatcgtagatgatggtacatacagcatttacttctctaaggaaggtatcctctctgcaattgacgatggcaagtctgagggaggtccatctgggcgacactgggtgctagatccaattgatgggactaaaggtttcttaaggggagaccaatatgctattgccctggctctgcttgatgagggtaaagttgttttgggtgtattggcttgtcccaacctttctttgggatcaataggcaaccttaatggtggctcctcgggagatcaagttggtgctctcttttctgctactattggttgtggagctgaagtagagtctttacagggctctccagcacaaaagattagtgtctgttccatcgacaatccagtcgaagcttcattctttgagtcctacgaaggggcacactccttgcgtgatttaacaggctccattgcggagaaacttggtgtccaagctcctccagttagaattgatagccaagcaaaatacggtgccctagcccgaggtgacggtgccatttacttgcgttttccacacaaaggttacagagagaagatctgggatcatgcagctgggtcaatcgtcgtgacagaagctggaggtctggtgacagatgcatcaggaaacgatttggatttctccaaagggagatttcttgatctcgacacagggatcatcgcgacgaacaagcagctgatgccttcactcctgaaggctgtgcaagatgccatcaaggagcaaaaccaggctgcttccccgttgtag

SEQ ID NO: 10 - protein, 358 aa, Oryza sativa
MSQAAGNPYAAELAAAKKAVTLAARLCQAVQKDILQSGVQSKADQSPVTVADYGSQILVSLVLKMEAPASSSFSMVAEEDSEELRKEGAEEILENITELVNETIVDDGTYSIYFSKEGILSAIDDGKSEGGPSGRHWVLDPIDGTKGFLRGDQYAIALALLDEGKVVLGVLACPNLSLGSIGNLNGGSSGDQVGALFSATIGCGAEVESLQGSPAQKISVCSIDNPVEASFFESYEGAHSLRDLTGSIAEKLGVQAPPVRIDSQAKYGALARGDGAIYLRFPHKGYREKIWDHAAGSIVVTEAGGLVTDASGNDLDFSKGRFLDLDTGIIATNKQLMPSLLKAVQDAIKEQNQAASPL

SEQ ID NO: 11 - DNA, 1257 bp, Oryza sativa
atgccgcccgggccgttcgtggcgcgcgacccgcgggtggtggcgctgctcgacgccgcctccttccccgtcctgttcgacacgtcgctcgtcgacaagaccgacctcttcaccggcaccttcatgccgtccaccgacctcaccggcgggtaccgcgtcctctcctaccttgacccctccgagcccaaccacgcgccgctcaagaccctcctcttctacctcctctcccaccggcggcagcaggtgatccccaagttccgcgaggtgtacggcgacctgttcggcctcatggagaacgacctcgcccgcgtcggcaaggccgacttcggcgtccacaacgacgccgccgcgttcggcttcctctgccaggggctcctcggccgcgacccggccaagtcggcgctggggcgcgacgggcccaagctgatcaccaagtgggtgctgttccagctcagcccgctgctcagcctcggcctccccaccctcgtcgaggacacgctcctccactcgctccgcctcccgccggcgctggtgaagaaggactacgaccgcctcgccgacttcttccgggacgcggccaaggccgtcgtcgacgagggggagcgcctcggcattgcacgggaggaggccgtgcacaacatcctcttcgcgctctgcttcaactcgttcggcgggatgaagatcctgttcccgacgctggtgaagtggctgggccgcgccggggcgcgtgtgcacgggcggctcgccaccgaggtgcgcggcgccgtgcgggacaacggcggggaggtgacgatgaaggcgctggcggagatgccgctggtgaagtcggcggtgtacgaggcgctgcggatcgagccgccggtggcgatgcagtacgggagggcgaagcgggacatggtggtggagagccacgactacgggtacgaggtgagggaaggggagatgctgttcgggtaccagcccatggcgaccaaggacccgcgggtgttcgcgcggccggaggagtacgtgccggacaggttcctcggcgaggacggcgcgcggctgctccgccacgtcgtgtggtccaacgggcccgagacggcggcgcccaccctgcacgacaagcagtgcgccggcaaggacttcgtcgtgctcgtcgcgcgcctcctcctcgtcgagctcttcctccgatacgactccttcgacgtcgaggtcggcacctccacgctcggctcatctgtcaccgtcacctcgctcaagaaggccaccttctga

SEQ ID NO: 12 - protein, 418 aa, Oryza sativa
MPPGPFVARDPRVVALLDAASFPVLFDTSLVDKTDLFTGTFMPSTDLTGGYRVLSYLDPSEPNHAPLKTLLFYLLSHRRQQVIPKFREVYGDLFGLMENDLARVGKADFGVHNDAAAFGFLCQGLLGRDPAKSALGRDGPKLITKWVLFQLSPLLSLGLPTLVEDTLLHSLRLPPALVKKDYDRLADFFRDAAKAVVDEGERLGIAREEAVHNILFALCFNSFGGMKILFPTLVKWLGRAGARVHGRLATEVRGAVRDNGGEVTMKALAEMPLVKSAVYEALRIEPPVAMQYGRAKRDMVVESHDYGYEVREGEMLFGYQPMATKDPRVFARPEEYVPDRFLGEDGARLLRHVVWSNGPETAAPTLHDKQCAGKDFVVLVARLLLVELFLRYDSFDVEVGTSTLGSSVTVTSLKKATF

SEQ ID NO: 13 - DNA, 1521 bp, Oryza sativa
atggagttcttcactgagtatggagaagcaagccagtaccagatccaggaagtcattggcaaaggaagttatggagtagttgctgctgcagtagatacccgcacgggtgagcgggttgcaatcaagaagatcaatgatgtgtttgagcatgtatcagacgctacgcgcatattgcgtgagatcaagctccttcgtctgctccgtcacccagacatagttgagatcaaacacattatgcttcccccttctcgaagggagttccaagatatttatgttgtttttgagctcatggaatcagatctccatcaagtcatcagagcgaacgatgacctcaccccggagcactaccagtttttcctgtaccaacttctccgtgctctcaagtacatccatgcagctaatgtatttcatcgcgatctaaagcccaagaatatactggcaaactcagactgcaaattgaaaatatgtgatttcggacttgcccgagcatcattcaatgatgccccttcagcaatattttggacggattatgttgcaacgaggtggtaccgagcacctgaattatgtggctcatttttctccaaatacactcctgcaattgatatttggagtattgggtgcatatttgctgaacttctcactgggagaccactatttcctgggaagaatgttgtgcaccaattagatattataacagatcttcttggaactccatcatcagaaaccttatccaggattcgaaatgagaaggccaggagatacttgagcaccatgcagaaaaaacatgctgtccccttctctcagaagttccgcaatactgaccccttggctcttcgtctgctagagcgtttactggcatttgatcctaaagatcggtcttcagctgaagaagctttggctgatccgtacttcgcaagtcttgctaatgtggaacgtgagccctcaagacatccaatctcaaaacttgagtttgaattcgagagacggaagctgacaaaagatgatgttagagaattaatttatcgagagattttggagtatcacccacagatgctgcaagagtatatgaaaggtggagagcagattagcttcctctatccaagtggggttgatcgcttcaaacgacagtttgcacaccttgaggagaactacagcaaaggagaaagaggttctccactgcagaggaagcatgcttctttaccgagggagagagtaggtgtatcaaaggatggttataaccaacaaaacaccaatgaccaagagaggagtgcagattccgttgcccgcactacagtaagccctccaatgtcacaagatgcacaacaacatggatctgctggccaaaatggtgtgacatccacagacttgagttcgaggagctatctgaagagtgcaagcattagtgcttccaagtgtgtcgctgtcaaggacaataaagaaccagaggatgattacatctctgaagaaatggaagggtcggtcgatggattgtctgaacaagtctccaggatgcactcctag

SEQ ID NO: 14 - protein, 506 aa, Oryza sativa
MEFFTEYGEASQYQIQEVIGKGSYGVVAAAVDTRTGERVAIKKINDVFEHVSDATRILREIKLLRLLRHPDIVEIKHIMLPPSRREFQDIYVVFELMESDLHQVIRANDDLTPEHYQFFLYQLLRALKYIHAANVFHRDLKPKNILANSDCKLKICDFGLARASFNDAPSAIFWTDYVATRWYRAPELCGSFFSKYTPAIDIWSIGCIFAELLTGRPLFPGKNVVHQLDIITDLLGTPSSETLSRIRNEKARRYLSTMQKKHAVPFSQKFRNTDPLALRLLERLLAFDPKDRSSAEEALADPYFASLANVEREPSRHPISKLEFEFERRKLTKDDVRELIYREILEYHPQMLQEYMKGGEQISFLYPSGVDRFKRQFAHLEENYSKGERGSPLQRKHASLPRERVGVSKDGYNQQNTNDQERSADSVARTTVSPPMSQDAQQHGSAGQNGVTSTDLSSRSYLKSASISASKCVAVKDNKEPEDDYISEEMEGSVDGLSEQVSRMHS

SEQ ID NO: 15 - DNA, 1185 bp, Oryza sativa
atggatcggccgccgccggatcagcagcggcagaagcaggcgccgctcttctcgccgtaccagatgccccgcttccgcctcaaccaccgggtggtgctggcgccgatgacgcggtgcagggcgatcggcggggtgcccggcccggcgctggcggagtactacgcgcagcggaccacccagggtggcctgctcatctccgagggcaccgtcgtctcgcccgctggcccggggtttcctcatgtccctgggatatacaatcaagagcagactgatgcatggaagaaggtggtggatgctgttcatgccaagggaggcatctttttctgccagttatggcatgtaggcagagcttctcaccaagtataccagccaaacggtgctgcaccaatatcctcaactgataagccaatatcagcaagatggagaatactgatgcctgatggctcctatggcaagtatcctaaacctaggcgcctggcagcatcggaaatacctgaaattgtcgaacaatatcgtcaagccgccattaatgccattgaagcaggttttgatggcattgagatccatggtgctcatggctatatcattgatcaattcctaaaggatggaatcaatgaccgcactgacgagtatggtggctcactttccaaccgctgccggttcctacttgaggtaactagggctgtggtttctgccattggagcagaccgagtcgcggtgaggatatcaccagccattgatcaccttgacgcctatgattcagaccccattaagctcggcatggccgttgttgagcggctgaatgctctccagcagcagtcagggcggctcgcctacctccacgtcacgcagccacggtacaccgcctacgggcagaccgagtctgggcagcatggcagtgccgaggaggagagccgcctgatgcgcaccctccggggcacgtaccagggcacattcatgtgcagtggcggctacacgcgggagcttgggttggaagcagtggagagcggcgatgccgacctggtgtcgtacgggcggctcttcatatcaaacccggacctggtcgagcggttcaggctgaacgccgggctgaacaagtacgtgcgtaagacattctacacgcccgatcctgtcgtgggttacacggactatccgttcctcggacagcctaagtcgcggatgtaa

SEQ ID NO: 16 - protein, 394 aa, Oryza sativa
MDRPPPDQQRQKQAPLFSPYQMPRFRLNHRVVLAPMTRCRAIGGVPGPALAEYYAQRTTQGGLLISEGTVVSPAGPGFPHVPGIYNQEQTDAWKKVVDAVHAKGGIFFCQLWHVGRASHQVYQPNGAAPISSTDKPISARWRILMPDGSYGKYPKPRRLAASEIPEIVEQYRQAAINAIEAGFDGIEIHGAHGYIIDQFLKDGINDRTDEYGGSLSNRCRFLLEVTRAVVSAIGADRVAVRISPAIDHLDAYDSDPIKLGMAVVERLNALQQQSGRLAYLHVTQPRYTAYGQTESGQHGSAEEESRLMRTLRGTYQGTFMCSGGYTRELGLEAVESGDADLVSYGRLFISNPDLVERFRLNAGLNKYVRKTFYTPDPVVGYTDYPFLGQPKSRM

SEQ ID NO: 17 - DNA, 678 bp, Oryza sativa
atgcattgctgcatgtcgcttcatcctcaccgccgccacggcgacggcgacgtcgacggatcagcatcaggatcaggatcagcgcgcctcaccgccggcctcatcaacttcctcgaatcgcgtcgcgccggcgccatgagcaccaccaacagctcatcctctgtctctgtcccagccatggacgcccatggacaggaggaggaggaggagccgatgcaggtgcagcaacagcaggcgttccgcggggtgcgcaagcggccatggggcaagtttgcggcggagatccgcgactcgacgcgcaacggcgtgcgcgtgtggctgggcacgttcgacagcgcggaggaggcggcgctggcctacgaccaggcggcgttcgccatgcgcgggtcggcggcggtgctcaacttccccatggagcaggtgaggcgttccatggacatgtccctcctgcaggaaggggcgtcgccggtggtggcgctgaagcggcggcactccatgcgagcggcagcagcggggcggcggcgcaagagcgctgcacctgcaccggcggatcaggaaggcggaggaggggtgatggagctggaggacctgggacctgactacctggaggagctgctagccgcctctcagcccatcgatatcacctgctgcacaagcccaagccaccactccatctga

SEQ ID NO: 18 - protein, 225 aa, Oryza sativa
MHCCMSLHPHRRHGDGDVDGSASGSGSARLTAGLINFLESRRAGAMSTTNSSSSVSVPAMDAHGQEEEEEPMQVQQQQAFRGVRKRPWGKFAAEIRDSTRNGVRVWLGTFDSAEEAALAYDQAAFAMRGSAAVLNFPMEQVRRSMDMSLLQEGASPVVALKRRHSMRAAAAGRRRKSAAPAPADQEGGGGVMELEDLGPDYLEELLAASQPIDITCCTSPSHHSI

SEQ ID NO: 19 - DNA, 588 bp, Oryza sativa
atgatctacccgccactgtaccgctgcaacgacgaggtgaagcagtgcgccgccgcctgcaaggagtgcgtggaggcgcccggcggcgacttcaacggcggcgccttcgtctgcagtgactggttctcgacggtggaccccggccccaagtgcacggcggcgctggatgggctgtcgatggagaggccgtggaagtgctgcgacaacatcaagcggctgccgacgaagcccgacccgccgcagtggcgctgcaacgacgagctggagcccagccagtgcaccgccgcgtgcaagtcgtgccgggaggcgccggggccattcccggggaagctcatctgcgaggacatctactggggcgccgacccgggccccttctgcacgccgcggccatggggagattgctacgacaaggccttctgcaacaagatgaacccgccgacctgccgctgcatggacgaggtgaaggagtgcgccgacgcgtgcaaggattgccagcgcgtggagtcgtcggagccgcctcgctacgtctgcaaggaccgcttcaccggccatcccggccccgtgtgcaaaccccgagcggagaactag

SEQ ID NO: 20 - protein, 195 aa, Oryza sativa
MIYPPLYRCNDEVKQCAAACKECVEAPGGDFNGGAFVCSDWFSTVDPGPKCTAALDGLSMERPWKCCDNIKRLPTKPDPPQWRCNDELEPSQCTAACKSCREAPGPFPGKLICEDIYWGADPGPFCTPRPWGDCYDKAFCNKMNPPTCRCMDEVKECADACKDCQRVESSEPPRYVCKDRFTGHPGPVCKPRAEN

SEQ ID NO: 21 - DNA, 2775 bp, Oryza sativa
atgttgcgtcctcagctcaatccatctagccacacgacgacgacgagcagcagcagcagcacgcagctgttcgcatcctcgtcgtgcatcgctagccttcgccggccgtcgtcgtcgtcgtcgtcggtggtcgccgccgcacgccggacgcgggggcaaggtagcagtcgggttgttgttgtgtgcgcgtcgtcgtcggcgacggcgagcaggggagatagttcttcggacatggcggcggcggcggcggtgcgggtgaaggcggtggcgacgatcaaggtcaccgtcggcgagttgatcaacaggtcgatcgacatcagggatctcatcggcaggtcgctctccctcgagctcgtcagctccgagcttgacgcgaagaccgggaaggagaaagcaactgtgcggagctacgcgcacaatgtggacgacgacgatcatagcgtcgtcacctacgaggccgacttcgacgtgccgagtggattcggccccatcggcgccatcatcgtcaccaacgaactccggcaggagatgttcctcgaggacatcaacctcaccgccagcgatggcgccggcaactccactgtcctccccatccgctgcaactcctgggtccaacccaagtccgtcggcgatgagggcacgcctagcaaacgcatcttcttcgccaacaagacttacttgccgggacagacgccggcggggctccggagctaccggaagaatgacctccagcagaagcgcggtgacggcactggcgagagggaggccgacgaccgtgtctacgactacgacgtttacaacgacctcggtaacccggacagcaacggcgatctcgcccgccccgtccttggcggcaacaagcagttcccctaccctcgccgctgccgcaccggccgccccccctccaaaaaagaccctaagtcggagacgaggaagggcaacgtgtacgtgccgagggacgaggagttctcaccggagaaggaggactacttcctccgcaagacggtggggtcggtgctccaggccgccgtgccggcggcgcagtcgctgctcctcgacaagctgaaatggaaccttccgttcccgtccttcttcgtcatcgacaagctgttcgaggacggcgtcgagcttcccggcgtcgacaagctcaacttcctcgagagcgtcgtgccccgcctgctcgaacacctccgcgacacccccgccgagaagatcctccgcttcgaaactccggccaacatccaaaaggacaagttcgcatggctcagagacgaggagttcgcgagggaaacgctcgctggcatcaacccgtacgccatcgagctcgtcagggaatttccgctgaagagcaagctcgacccggcggtgtacggtccggcggagtcggcgatcaccgccgatttgctggaggagcagatgaggcgcgtgatgacggtggaggaggcgatcagccagaagaggctgttcatgctcgacttccatgacctcttcttgccgtacgtgcacaagatccggtcgctggatcacaccaccatgtacggctcgcgcaccgtcttcttcctcaccgacgacggcacgctgcagctgctcgccatcgagctcacccggccggcctcgccgtcgcagccgcagtggcggcaggtgttcacgccgtccacggacgccaccatgtcgtggctgtggcggatggccaaggcccacgtccgcgcccacgacgccggccaccacgagctcatcacccactggctgcgcacgcactgcgcggtggagccatacatcatcgcggcgaaccggcagctcagcgagatgcaccccatctaccagctgctgcgcccgcacttccgctacacgatgcggatcaacgcgcgcgcccgctcggcgctgatcagcgccggcggcatcatcgagcgatccttctcgccgcagaagtactccatggagctcagctccgtcgcctacgacaagctctggcgcttcgacacggaggcgctccccgccgacctcgtccgccgcggcatggccgaggaggaccccacggcggagcacggcctcaagctcgccatcgaggactacccgttcgccaacgacggcctcctcatctgggacgccatcaagacctgggtccaggcgtacgtcgcgcggttctaccccgacgccgacagcgtcgccggcgacgaggagctccaggcgttctggaccgaggtgcgcaccaaggggcacggcgacaagaaggacgccccgtggtggccgaagttggactcgccggagagcctcgcccacacgctgaccaccatcgtctgggtggcggcggcgcaccacgccgccgtcaacttcgggcagtacgacttcggcggctacttccccaaccggccgtccatcgcgcgcacggtcatgccggtggaggagcccgtggacggcgccgccatggagaggttcctggacaacccggaccaggcgctccgcgagtgcttcccgtcacaggtgcaggcgacggtggtgatggcggtgctcgacgtgctgtccagccactccaccgacgaggagtacctcggcggcgagcagacgaggccgtggaacagcgacgcggcggtgcaggcggcgtacgacgggttcgcagcccggctcaaggagatcgagggcgtcatcgatggccggaacaaggatagaaagctcaagaacaggtgcggcgccggcatcctgccgtaccagctgatgaagcccttctccgactccggcgtcaccggcatgggcatccccaacagcacatccatctga

SEQ ID NO: 22 - protein, 924 aa, Oryza sativa
MLRPQLNPSSHTTTTSSSSSTQLFASSSCIASLRRPSSSSSSVVAAARRTRGQGSSRVVVVCASSSATASRGDSSSDMAAAAAVRVKAVATIKVTVGELINRSIDIRDLIGRSLSLELVSSELDAKTGKEKATVRSYAHNVDDDDHSVVTYEADFDVPSGFGPIGAIIVTNELRQEMFLEDINLTASDGAGNSTVLPIRCNSWVQPKSVGDEGTPSKRIFFANKTYLPGQTPAGLRSYRKNDLQQKRGDGTGEREADDRVYDYDVYNDLGNPDSNGDLARPVLGGNKQFPYPRRCRTGRPPSKKDPKSETRKGNVYVPRDEEFSPEKEDYFLRKTVGSVLQAAVPAAQSLLLDKLKWNLPFPSFFVIDKLFEDGVELPGVDKLNFLESVVPRLLEHLRDTPAEKILRFETPANIQKDKFAWLRDEEFARETLAGINPYAIELVREFPLKSKLDPAVYGPAESAITADLLEEQMRRVMTVEEAISQKRLFMLDFHDLFLPYVHKIRSLDHTTMYGSRTVFFLTDDGTLQLLAIELTRPASPSQPQWRQVFTPSTDATMSWLWRMAKAHVRAHDAGHHELITHWLRTHCAVEPYIIAANRQLSEMHPIYQLLRPHFRYTMRINARARSALISAGGIIERSFSPQKYSMELSSVAYDKLWRFDTEALPADLVRRGMAEEDPTAEHGLKLAIEDYPFANDGLLIWDAIKTWVQAYVARFYPDADSVAGDEELQAFWTEVRTKGHGDKKDAPWWPKLDSPESLAHTLTTIVWVAAAHHAAVNFGQYDFGGYFPNRPSIARTVMPVEEPVDGAAMERFLDNPDQALRECFPSQVQATVVMAVLDVLSSHSTDEEYLGGEQTRPWNSDAAVQAAYDGFAARLKEIEGVIDGRNKDRKLKNRCGAGILPYQLMKPFSDSGVTGMGIPNSTSI

SEQ ID NO: 23 - DNA, 1797 bp, Oryza sativa
atgggcacgtacaagtgctgcctcatcttcaagcgccgcttccgctggaacgacgcgccgccgcccgacgatgtccgcgccctcttcgccaaccactccgccggcggtggcccccacatggccgccgacggcctccgcgcctacctccaggccaccggccaggacggcgacgtggacatggagcggctggtggagcagatccggcagctgcaggggcgcggcgggcgcatcccgcgggtggggcgggcactcccactcctgacggtggacgacttccaccgattcctcttctcccacgagctgaacccacccatccggcacgggcaggggcaggtgcaccacgacatggccgccccgctctcccactacttcatctacaccggccacaactcctacctcaccggcaaccagctcagcagcgactgcagcgacctccccatcatcagggctctccagaggggcgtccgcgtcatcgagctcgacatgtggcccaactcctccaaggatgacatcagcatcctccatggcaggacgctcaccaccccggtctccctcctcaaatgcctcctctccatcaagcaacacgcctttgaggcctccccttacccggttatcatcacgctcgaagaccacctcacccccgatctccaggacaaagcagccaagatggttcttgaagttttcggcgacatcctctactaccctgacaaagatcacctcaaagagttcccttcgcctcaagacctcaagggccgtgtcctcctctccaccaagccccccagggagtaccttcaagccaaggatggtaatgctgccaccatcaaagaggacgccaaggccgccgccactgacgatgccgcatggggaaaagaagtcccagatattcactctcaaatccactctgccactaaacatgaccaaagagaagatgacgacgacaccgatgaagacgaagatgacgaggaggaggagcagaaaatgcaacagcatctagctccacagtacaaacaccttattaccatcaaagcaggaaagccaaaaggtactctacttgatgccttacagagtgacccagaaaaggttagaaggctcagtttgagcgagcaacaacttgccaaattggcagatcatcatggtaccgaaattgtaaggttcacacagagaaacctactgaggatatacccaaagggcactcgggtcacatcatccaactataatccatttcttggttgggtgcatggtgctcagatggtagcgttcaatatgcagggatatggaagagctctttggttgatgcatggattttataaagctaatggtggctgtggttatgtgaagaaaccagatttcttaatgcaaactgatccagaggtttttgacccaaaaaaatccctatctcccaagaaaaccttgaaggtgaaagtatacatgggggatggttggcggatggacttcacgcagacccactttgatcaatattctcctccagacttttatgcacgggtggggatagcgggagtaccagcggactcggtgatgaagagaacgagggcgatagaggataactgggtgccggtgtgggaggaggatttcaccttcaaactgaccgtgccggagatcgcgttgctgcgggtggaggtgcacgagtacgacatgtcggagaaggacgacttcggcggccagacggtgctgccggtgtcggatctcatcccggggatccgagcggtggcactccacgaccgcaaagggatcaagttgaacaacgtcaagcttctcatgcgcttcgagtttgaatga

SEQ ID NO: 24 - protein, 598 aa, Oryza sativa
MGTYKCCLIFKRRFRWNDAPPPDDVRALFANHSAGGGPHMAADGLRAYLQATGQDGDVDMERLVEQIRQLQGRGGRIPRVGRALPLLTVDDFHRFLFSHELNPPIRHGQGQVHHDMAAPLSHYFIYTGHNSYLTGNQLSSDCSDLPIIRALQRGVRVIELDMWPNSSKDDISILHGRTLTTPVSLLKCLLSIKQHAFEASPYPVIITLEDHLTPDLQDKAAKMVLEVFGDILYYPDKDHLKEFPSPQDLKGRVLLSTKPPREYLQAKDGNAATIKEDAKAAATDDAAWGKEVPDIHSQIHSATKHDQREDDDDTDEDEDDEEEEQKMQQHLAPQYKHLITIKAGKPKGTLLDALQSDPEKVRRLSLSEQQLAKLADHHGTEIVRFTQRNLLRIYPKGTRVTSSNYNPFLGWVHGAQMVAFNMQGYGRALWLMHGFYKANGGCGYVKKPDFLMQTDPEVFDPKKSLSPKKTLKVKVYMGDGWRMDFTQTHFDQYSPPDFYARVGIAGVPADSVMKRTRAIEDNWVPVWEEDFTFKLTVPEIALLRVEVHEYDMSEKDDFGGQTVLPVSDLIPGIRAVALHDRKGIKLNNVKLLMRFEFE

SEQ ID NO: 25 - DNA, 1140 bp, Oryza sativa
atggcagcgacgatggtggcgatgtcggccaagagcaagaacagcgtgctgacattggagaagaagcagggctggtctgtcccacaacttccggagctccggttcccttgggatttgcatgaagacaagggcttctctttgagcttacatggctctgcctcccctcatggtgggctgtttgctagcgtcggtctcaaagtgtccacagctgcaccagcagtggcacccagtcctgccgagcatgacttcaagattccgttcgctgatcattgcataaagtatgtctcctcagcagtggggtaccaagttcctgggactgaggctgaatctgtcaatgaggaagaggtggtggatggcaaggctgtgaagaaagccaagaaacgtgggctgaagctgaaaattaagattgggaacccacatttgaggcggctggttagcggagccgttgcgggagctgtctcgaggacttgtgtggcacctctggagacgattaggacccatttgatggttgggagcaatggggactctatgacagaggtattccagtcaatcatgaagaccgaggggtggacagggctgttccgtgggaactttgtcaatgtcatccgagttgcaccaagcaaggctattgagctatttgctttcgatacagccaagaaattcttaactccaaaggctgatgagtcccctaagacacccttccctccatcgcttattgctggagcacttgctggggttagctcaacattgtgcacataccccttggaattgatcaagacccgattgactattgagaaagatgtctataacaacttcctccatgctttcgtcaagatactacgagaggaaggcccctcagctctaccgcgggctgacaccgagtctgatcggcgtggtgccatacgctgcaaccaattactatgcctacgacaccctgaagaagctctacaggaagacattcaagcaggaggagatcagcaacatcgcgactcttctcatcggttcagccgcgggtgccatctcgagcaccgccaccttccctctcgaggtagctcgcaagcaaatgcaggtcggagcggtaggcggcaggcaggtctacaagaatgtcttccatgctctgtattgcataatggagaatga

SEQ ID NO: 26 - protein, 379 aa, Oryza sativa
MAATMVAMSAKSKNSVLTLEKKQGWSVPQLPELRFPWDLHEDKGFSLSLHGSASPHGGLFASVGLKVSTAAPAVAPSPAEHDFKIPFADHCIKYVSSAVGYQVPGTEAESVNEEEVVDGKAVKKAKKRGLKLKIKIGNPHLRRLVSGAVAGAVSRTCVAPLETIRTHLMVGSNGDSMTEVFQSIMKTEGWTGLFRGNFVNVIRVAPSKAIELFAFDTAKKFLTPKADESPKTPFPPSLIAGALAGVSSTLCTYPLELIKTRLTIEKDVYNNFLHAFVKILREEGPSALPRADTESDRRGAIRCNQLLCLRHPEEALQEDIQAGGDQQHRDSSHRFSRGCHLEHRHLPSRGSSQANAGRSGRRQAGLQECLPCSVLHNGE

SEQ ID NO: 27 - DNA, 1488 bp, Oryza sativa
atgccgtggccgcacgtgctgacggtggccggctccgactccggcggcggcgccggcatccaggccgacatcaaggcctgcgccgcgctgggagcctactgctcctccgtcgtcaccgccgtcactgcccagaacaccgccggcgtccaggggactcatgtggtgccagaggagttcatccgggagcagctcaactcggttctttcggacatgtcggtggatgtggtcaagacaggaatgctcccttcaatcggagtagtcggagttttatgtgaaagtctaaagaagtttccagtcaaagctttagtggtggatccagttatggtgtccacaagtggagatactctttcagaatcatctactctctctgtatatagggatgaattatttgctatggctgatatagtcaccccaaatgtgaaagaagcgtcgagattacttgggggtgtatctttgcgcactgtttctgacatgcgcaatgcagcggagtccatttacaaatttggtccaaaacatgtacttgtgaaaggtggggatatgctagaatcctcagatgcaactgatgtattttttgatggtaaggagttcattgagctccatgcgcatcgcataaagacccataacacacatggaactggttgcactttagcttcatgtatagcttccgaattagcgaaaggtgcaacgatgctgcatgctgttcaggtggctaagaattttgtagaatctgctcttcatcacagtaaagatcttgtcgttggaaatggacctcaaggcccttttgatcaccttttcaagctcaagtgtccgccatacaatgttggctcacagccaagctttaagccagaccaactcttcctgtatgcagtgacagactctgggatgaacaaaaagtggggtcgttcaatcaaagaagctgtgcaagctgccattgaaggtggcgctaccattgtacaactgagagaaaaagattccgaaacaagagagtttttggaagcagccaaagcatgcatggagatttgcaagtccagtggagtaccactgctgatcaacgaccgggtagacattgccctggcacgcaatgcagatggtgtccatgttggtcaattggacatgtcggcacacgaagtgcgggagctcctagggccgggtaaaatcatcggcgtctcgtgcaagacccctgctcaagcccagcaggcctggaatgatggagcagactacattggctgtggcggtgttttccctacctccacgaaggcgaacaatcctacattggggtttgatggcttgaagacagtttgcctggcatctaagctgcctgtggtcgctattggcggcatcaacgcttcaaatgcaggttcagtgatggagcttggtctcccaaacctcaaaggcgtcgcagtagtctccgctctgtttgaccgtccgagtgtcgtagccgaaacaagaaacatgaaatccatcttgaccaacacctctagaacctag

SEQ ID NO: 28 - protein, 495 aa, Oryza sativa
MPWPHVLTVAGSDSGGGAGIQADIKACAALGAYCSSVVTAVTAQNTAGVQGTHVVPEEFIREQLNSVLSDMSVDVVKTGMLPSIGVVGVLCESLKKFPVKALVVDPVMVSTSGDTLSESSTLSVYRDELFAMADIVTPNVKEASRLLGGVSLRTVSDMRNAAESIYKFGPKHVLVKGGDMLESSDATDVFFDGKEFIELHAHRIKTHNTHGTGCTLASCIASELAKGATMLHAVQVAKNFVESALHHSKDLVVGNGPQGPFDHLFKLKCPPYNVGSQPSFKPDQLFLYAVTDSGMNKKWGRSIKEAVQAAIEGGATIVQLREKDSETREFLEAAKACMEICKSSGVPLLINDRVDIALARNADGVHVGQLDMSAHEVRELLGPGKIIGVSCKTPAQAQQAWNDGADYIGCGGVFPTSTKANNPTLGFDGLKTVCLASKLPVVAIGGINASNAGSVMELGLPNLKGVAVVSALFDRPSVVAETRNMKSILTNTSRT

SEQ ID NO: 29 - DNA, 1143 bp, Oryza sativa
atggcgtcgctgctgatcgccagcacggggggcgcccaccgcctcgcgtggaaggacgccgccgccctgggacccgctccgcggctggcgcgaccttggcccgccgccgtggctgcaccggcgccgctgctcaggattagcagaggaaagtttgcattgcaggccatcacccttgatgattatcttccaatgcgaagtactgaagtgaaaaatcggacatcaacagctgatatcactagtctcagagtaattacagcggtcaaaaccccatatctgcctgatggaagatttgatcttgaagcatatgattcactgataaatatgcagatagatggtggtgctgaaggtgtaatagttggaggaacaacaggagagggccaccttatgagctgggatgaacacatcatgcttattggacatactgttaactgctttggtgctaaagttaaagtggtaggcaacacaggtagtaactcaacaagagaggctattcatgcaacagagcagggatttgctgtaggtatgcatgcggctctccatatcaatccttactatgggaagacctctatcgaagggttgatatctcattttgaggctgtcctcccaatgggtccaaccattatttacaatgttccatctaggactggccaggatattcctcctgcagttattgaggctgtttcaagtttcacaaacttggcaggtgtgaaagaatgtgttggacatgagagggttaagtgctacactgacaaaggtataaccatatggagtggtaatgatgatgaatgccatgattctaggtggaaatatggtgccactggagttatttctgtggctagcaaccttattcctggtctcatgcacgatctcatgtatgaaggggagaataagacgctaaatgagaagctctttcccctgatgaaatggttgttttgccagccaaatccaattgctctcaacactgccctggctcagcttggagtggtaaggcctgttttcagattaccatatgtacctcttcctcttgaaaagagggtagagtttgtccgaatcgttgaatctattggacgggaaaactttgtgggtgagaacgaggcacgggttcttgacgacgatgattttgtgttggtcagtaggtactaa

SEQ ID NO: 30 - protein, 380 aa, Oryza sativa
MASLLIASTGGAHRLAWKDAAALGPAPRLARPWPAAVAAPAPLLRISRGKFALQAITLDDYLPMRSTEVKNRTSTADITSLRVITAVKTPYLPDGRFDLEAYDSLINMQIDGGAEGVIVGGTTGEGHLMSWDEHIMLIGHTVNCFGAKVKVVGNTGSNSTREAIHATEQGFAVGMHAALHINPYYGKTSIEGLISHFEAVLPMGPTIIYNVPSRTGQDIPPAVIEAVSSFTNLAGVKECVGHERVKCYTDKGITIWSGNDDECHDSRWKYGATGVISVASNLIPGLMHDLMYEGENKTLNEKLFPLMKWLFCQPNPIALNTALAQLGVVRPVFRLPYVPLPLEKRVEFVRIVESIGRENFVGENEARVLDDDDFVLVSRY

SEQ ID NO: 31 - DNA, 768 bp, Oryza sativa
atgcttcctcctagggttgccccctcctctctggccgccgccgccgccgcggcgcctacctatctcgccgccgcggcctcgacccctgcttccgtctggctgcctgtgccgcgtggagccggagccgtggcagtgtgcagggccgccgggaaagggaaggaggtgctcagcggcgtggtcttccagccattcgaggagctcaagggggagctctccctcgtcccccaggccaaggaccagtctcttgctaggcaaaagttcgtcgacgagtgcgaggccgccatcagcgagcagatcaatgtggagttcaatgcatcgtacgcgtaccactcccttttcgcctactttgaccgtgacaacgttgctctcaagggattcgccaaattcttcaaagaatccagcgatgaggagagggatcacgcagagaaactcatgaagtaccagaacatgcgtggaggcagggtgcggctccagtccatcgtcacacccttgacagagttcgaccatcctgagaaaggggatgccttgtatgctatggagttggccttggctctcgaaaagcttgtaaatgagaagttgcacaacctgcacagtgtggcatcaaggtgcaatgatccacagctgaccgacttcgttgagagtgaattccttgaggagcaggttgaagccatcaagaagatctctgagtatgtcgcccagctgagaagagtgggaaaggggcatggggtgtggcactttgatcagaagctgcttgaggaagaagcttga

SEQ ID NO: 32 - protein, 255 aa, Oryza sativa
MLPPRVAPSSLAAAAAAAPTYLAAAASTPASVWLPVPRGAGAVAVCRAAGKGKEVLSGVVFQPFEELKGELSLVPQAKDQSLARQKFVDECEAAISEQINVEFNASYAYHSLFAYFDRDNVALKGFAKFFKESSDEERDHAEKLMKYQNMRGGRVRLQSIVTPLTEFDHPEKGDALYAMELALALEKLVNEKLHNLHSVASRCNDPQLTDFVESEFLEEQVEAIKKISEYVAQLRRVGKGHGVWHFDQKLLEEEA

SEQ ID NO: 33 - DNA, 756 bp, Oryza sativa
atgcttcctcctagggttgccccggccgccgccgccgccgcgcctacctatctcgccgccgcggcctcgacccctgcttccgtctggctgcctgtgccgcgtggtgccggacccggggcagtgtgcagggccgccgggaaagggaaggaggtgctcagcggcgtggtcttccagccattcgaggagctcaagggggagctctccctcgtcccccaggccaaggaccagtctctcgctaggcaaaagttcgtcgacgagtgcgaggccgccatcaacgagcagatcaatgtggagtacaatgcatcgtacgcgtaccactcccttttcgcctactttgatcgtgacaacgttgctctcaagggattcgccaaattcttcaaagaatccagcgatgaggagagggatcacgcagagaaactcatcaagtaccagaacatgcgtggaggcagggtgcggctccagtccatcgtcacacctttgacagagttcgaccatcctgagaaaggggatgccttgtatgctatggagttggccttggctctcgaaaagcttgtaaatgagaagttgcacaacctgcacagtgtggcatcaaggtgcaatgatccacagctgaccgacttcgttgagagcgaattccttgaggagcaggttgaagccatcaagaagatctctgagtatgtcgcccagctgagaagagtgggaaaggggcatggggtgtggcactttgatcagaagctgcttgaggaagaagcttga

SEQ ID NO: 34 - protein, 251 aa, Oryza sativa
MLPPRVAPAAAAAAPTYLAAAASTPASVWLPVPRGAGPGAVCRAAGKGKEVLSGVVFQPFEELKGELSLVPQAKDQSLARQKFVDECEAAINEQINVEYNASYAYHSLFAYFDRDNVALKGFAKFFKESSDEERDHAEKLIKYQNMRGGRVRLQSIVTPLTEFDHPEKGDALYAMELALALEKLVNEKLHNLHSVASRCNDPQLTDFVESEFLEEQVEAIKKISEYVAQLRRVGKGHGVWHFDQKLLEEEA

SEQ ID NO: 35 - DNA, 1662 bp, Oryza sativa
atgtatcgggtgaagagcgagagcgactgcgatatgatccatcaggagcagatggactcgccggtggccgacgacggcagcagcggggggtcgccgcaccgcggcggcgggcccccgctgaagaaggggccatggacgtcggcggaggacgccatcctggtggactacgtgaagaagcacggcgaggggaactggaacgcggtgcagaagaacaccgggctgttccggtgcggcaagagctgccgcctccggtgggcgaaccacctgaggcccaacctcaagaagggggccttcaccgccgaggaggagaggctcatcatccagctccactccaagatggggaacaagtgggctcggatggccgctcatttgccagggcgcactgataatgaaataaagaattactggaatactcgaataaagagatgccagcgagctggcctacccatctatcctaccagcgtatgcaatcaatcctcaaatgaagatcagcagtgctccagtgattttgactgtggcgagaatttgtcaaacgatcttctgaatgcaaatggtctttacctaccagattttacctgtgacaatttcattgctaattcagaggctttaccttatgcaccacatctttcagccgtttctataagcaatctccttggccagagctttgcatcaaaaagctgtagcttcatggatcaggtaaaccagacagggatgctaaaacagtctgatggtgtgcttcctggattgagcgataccatcaacggtgtgatttcctcggtggatcaattctcaaatgactctgagaagctcaagcaggctgtgggttttgactatctccatgaagccaactctaccagcaagattattgcacctttcgggggtgcacttaatggcagccatgcctttttaaatggcaatttctctgcttctaggcccacaagtggtcctttgaagatggagctcccttcactccaagatactgaatctgatccaaacagctggctcaagtacactgtagctcctgcgttgcagcctactgagttagttgatccctacctgcagtctccagcagcaaccccttcagtgaaatcagagtgcgcgtcgccaaggaatagtggccttttggaagagttgattcatgaagctcggaccctaagatccgggaagaaccaacagacatctgtgataagttctagttcttctgtcggtacgccatgtaatactacggttcttagcccagagtttgatatgtgtcaggaatactggggagaacaacatcctggtccattcctcaatgactgtgctcctttcagtggcaattcattcactgaatccacccctcctgttagcgctgcatcgcctgacatctttcagctctccaaagtttccccagcacaaagcacttcaatgggatctggagagcaagtaatggggcctaaatatgaacctggggacacttcacctcatcctgaaaacttcaggccagatgcattgttttctgggaatacagctgatccatcagttttcaacaatgccatagcaatgcttctgggcaatgacttgagtatcgattgcagacctgttcttggcgacggtatcatgttcaattcttcctcgtggagcaacatgccacacgcctgtgaaatgtcagaattcaaatga

SEQ ID NO: 36 - protein, 553 aa, Oryza sativa
MYRVKSESDCDMIHQEQMDSPVADDGSSGGSPHRGGGPPLKKGPWTSAEDAILVDYVKKHGEGNWNAVQKNTGLFRCGKSCRLRWANHLRPNLKKGAFTAEEERLIIQLHSKMGNKWARMAAHLPGRTDNEIKNYWNTRIKRCQRAGLPIYPTSVCNQSSNEDQQCSSDFDCGENLSNDLLNANGLYLPDFTCDNFIANSEALPYAPHLSAVSISNLLGQSFASKSCSFMDQVNQTGMLKQSDGVLPGLSDTINGVISSVDQFSNDSEKLKQAVGFDYLHEANSTSKIIAPFGGALNGSHAFLNGNFSASRPTSGPLKMELPSLQDTESDPNSWLKYTVAPALQPTELVDPYLQSPAATPSVKSECASPRNSGLLEELIHEARTLRSGKNQQTSVISSSSSVGTPCNTTVLSPEFDMCQEYWGEQHPGPFLNDCAPFSGNSFTESTPPVSAASPDIFQLSKVSPAQSTSMGSGEQVMGPKYEPGDTSPHPENFRPDALFSGNTADPSVFNNAIAMLLGNDLSIDCRPVLGDGIMFNSSSWSNMPHACEMSEFK

SEQ ID NO: 37 - DNA, 1089 bp, Oryza sativa
atggcccacgccgccgcggccacgggcgcactggcaccgctgcatccactgctccgctgcacgagccgtcatctctgcgcctcggcttcccctcgcgccggcctctgcctccaccaccaccgccgccgccgccgcagcagccggaggacgaaactcgccgtgcgcgcgatggcaccgacgttgtcctcgtcgtcgacggcggcggcagctcccccggggctgaaggagggcatcgcggggctctacgacgagtcgtccggcgtgtgggagagcatctggggcgagcacatgcaccacggcttctacgacgccggcgaggccgcctccatgtccgaccaccgccgcgcccagatccgcatgatcgaggaatccctcgccttcgccgccgtccccgatgatgcggagaagaaacccaaaagtgtagttgatgttggctgtggcattggtggtagctcaagatacttggcgaacaaatacggagcgcaatgctacggcatcacgttgagtccggtgcaggctgaaagaggaaatgccctcgcggcagagcaagggttatcagacaaggtctcctttcaagttggtgatgcattggagcagccttttcctgatgggcagtttgatcttgtctggtccatggagagtggcgagcacatgccagacaaacggcagtttgtaagcgagctggcacgcgtcgcagctcctggggcgagaataatcattgtgacctggtgccataggaacctcgagccatccgaagagtccctgaaacctgatgagctgaatctcctgaaaaggatatgcgatgcatattatctcccagactggtgctctccttctgattatgtcaaaattgccgagtcactgtctcttgaggatataaggacagctgattggtcagagaacgtcgccccattctggcctgcggttataaaatcagcattgacatggaaaggtttaacttctctgctaagaagtgggtggaagacgataagaggtgcaatggtgatgcctctgatgatcgaaggatacaagaaagggctcatcaaattcaccatcatcacctgtcgcaagcccgaaacaacgcagtag

SEQ ID NO: 38 - protein, 362 aa, Oryza sativa
MAHAAAATGALAPLHPLLRCTSRHLCASASPRAGLCLHHHRRRRRSSRRTKLAVRAMAPTLSSSSTAAAAPPGLKEGIAGLYDESSGVWESIWGEHMHHGFYDAGEAASMSDHRRAQIRMIEESLAFAAVPDDAEKKPKSVVDVGCGIGGSSRYLANKYGAQCYGITLSPVQAERGNALAAEQGLSDKVSFQVGDALEQPFPDGQFDLVWSMESGEHMPDKRQFVSELARVAAPGARIIIVTWCHRNLEPSEESLKPDELNLLKRICDAYYLPDWCSPSDYVKIAESLSLEDIRTADWSENVAPFWPAVIKSALTWKGLTSLLRSGWKTIRGAMVMPLMIEGYKKGLIKFTIITCRKPETTQ

SEQ ID NO: 39 - DNA, 1161 bp, Oryza sativa
atggcttgctccacccacctcctgtcccagagcctctacccgctcaaccgcgctaacccggcagccgcgcgcgggcacctccggttccaggcttctcccagtgtgaggctcggctccggcaccagtcgccgccgggcgctgggcctgagagtcgccgcgtcggcggagcaagggaggaggcaagtggaggttgagtatgatctacaagcaaagttcaacaagctagcggaccaaattgaccagaatgctgggattacacggctgaacctattctcaccttgcaaaattaatgtgttcttgaggataacgggtaagagaccagatgggttccatgacctggcttctctgtttcatgtgataagtttgggtgatactatcaaattctcattgtcaccaagtaagagcaaagatcgcttgtcaaccaatgtagcaggtgtcccagttgatgaaagcaatttgatcatcaaggcactcaatctttaccgcaagaaaactggaactgacaactttttttggattcatcttgataagaaggtccctactggtgctggtcttggtggtggaagcagtaatgctgcaactgcgctgtgggccgccaaccagtttagtggctgcattgcttcagaaaaggagcttcaggagtggtctggagagataggatcggatattcccttcttcttttcacaaggagcagcgtattgtaccggtagaggagagattgttgaagatattcggaatccattgccagcaaatttgccgatggtactagtaaagccacctgaagcatgctcaacagctgaagtttacaagcggctcaggttagagcacacaagtcaaactgatcccttggtattgctcaaggaaattactgaaaatgggatatcacaggatgcctgtgttaatgatctagaacctccagcatttgaggtgttgccatcactaaagaggttgaagaaacgtataattgctgctaaccggggagattatgatgctgtttttatgtcaggaagtggaagcacaattgttgggattggttcaccagatccgcctgcatttgtgtatgatgatgatgactacaaggatacttttgtgtcagaggcctgcttcctcactcgtaatgagaacgagtggtacagaaccaatctcatcgaaaatcactag

SEQ ID NO: 40 - protein, 386 aa, Oryza sativa
MACSTHLLSQSLYPLNRANPAAARGHLRFQASPSVRLGSGTSRRRALGLRVAASAEQGRRQVEVEYDLQAKFNKLADQIDQNAGITRLNLFSPCKINVFLRITGKRPDGFHDLASLFHVISLGDTIKFSLSPSKSKDRLSTNVAGVPVDESNLIIKALNLYRKKTGTDNFFWIHLDKKVPTGAGLGGGSSNAATALWAANQFSGCIASEKELQEWSGEIGSDIPFFFSQGAAYCTGRGEIVEDIRNPLPANLPMVLVKPPEACSTAEVYKRLRLEHTSQTDPLVLLKEITENGISQDACVNDLEPPAFEVLPSLKRLKKRIIAANRGDYDAVFMSGSGSTIVGIGSPDPPAFVYDDDDYKDTFVSEACFLTRNENEWYRTNLIENH

SEQ ID NO: 41 - DNA, 1278 bp, Oryza sativa
atggagcgggtgttctccgtggaggagatctccgacccattctgggtcccgcctccgccgccgcagtcggcggcggcggcccagcagcagggcggcggcggcgtggcttcgggaggtggtggtggtgtagcggggggcggcggcggcgggaacgcgatgaaccggtgcccgtcggagtggtacttccagaagtttctggaggaggcggtgctcgatagccccgtcccgaaccctagcccgagggccgaagcgggagggatcaggggcgcaggaggggtggtgccggtcgatgttaagcagccgcagctctcggcggcggcggcggcggcggcgacgacgagcgcggtggtggaccccgtggagtacaacgcgatgctgaagcagaagctggagaaggacctcgccgcggtcgccatgtggagggcctctggcacagttccacctgagcgtcctggagctggttcatccttgctgaatgcagatgtttcacacataggcgctcctaattccatcggaggcaatgctactccagttcaaaacatgctaagtggcccaagtgggggatcgggctcacagttggtacagaatgttgatgtccttgtaaagcagcccaccagctcttcatcaagggagcagtcagatgatgatgacatggagggagaagctgagaccactggaactgcaagacctgctgatcaaagattacaacgaaggaagcaatccaatcgggagtcagccaggcgctcaagaagcagaaaggcagctcacttgaatgagctggaggcacaggtatcgcaattaagagtcgagaactcctcgctgttaaggcgtcttgctgatgttaaccagaagtacaatgatgctgctgttgacaatagagtgctaaaagcagatgttgagaccttgagagcaaaggtgaagatggcagaggactcggtgaagcgggtgacaggcatgaacgcgttgtttcccgccgcttctgatatgtcatccctcagcatgccattcaacagctccccatctgaagcaacgtcagacgctgctgttcccatccaagatgacccgaacaattacttcgctactaacaacgacatcggaggtaacaacaactacatgcccgacataccttcttcggctcaggaggacgaggacttcgtcaatggcgctctggctgccggcaagattggccggacagcctcgctgcagcgggtggcgagcctggagcatctccagaagaggatgtgcggtgggccggcttcgtctgggtcgacgtcctga

SEQ ID NO: 42 - protein, 425 aa, Oryza sativa
MERVFSVEEISDPFWVPPPPPQSAAAAQQQGGGGVASGGGGGVAGGGGGGNAMNRCPSEWYFQKFLEEAVLDSPVPNPSPRAEAGGIRGAGGVVPVDVKQPQLSAAAAAAATTSAVVDPVEYNAMLKQKLEKDLAAVAMWRASGTVPPERPGAGSSLLNADVSHIGAPNSIGGNATPVQNMLSGPSGGSGSQLVQNVDVLVKQPTSSSSREQSDDDDMEGEAETTGTARPADQRLQRRKQSNRESARRSRSRKAAHLNELEAQVSQLRVENSSLLRRLADVNQKYNDAAVDNRVLKADVETLRAKVKMAEDSVKRVTGMNALFPAASDMSSLSMPFNSSPSEATSDAAVPIQDDPNNYFATNNDIGGNNNYMPDIPSSAQEDEDFVNGALAAGKIGRTASLQRVASLEHLQKRMCGGPASSGSTS

SEQ ID NO: 43 - DNA, 831 bp, Oryza sativa
atgacgggcagtagttgtccggccgattcttcccagctgctgtaccctcgccggggtgcccccaccaccaccaccacctcccgtcctcctctccatccgctcatcgctcatgcgccctacgacgtcgtcctccaccgatctgtcgtcctctccatcagctcagctcgtgatcaggctgagctcgcgagcttctggtgctatataaggctgggtggtggtgcaagtgcgaagcgagcggccggtgaggacgatcgatcgagatcgagcttgacggcggcgagaggaggaggaggggagacgatgagcgggcgggggaggggggacccgctggtgctggggagggtggtgggggacgtggtggacccgttcgtgaggagggtggcgctgcgggtggcgtacggagcgcgggaggtggccaacggctgcgagctccgcccctccgccgtcgccgaccagccccgcgtcgccgtcggcggccccgacatgcgcaccttctacaccctggtgatggtggatccggacgcgccgagcccgagcgatccaaacctcagggagtacctgcactggctggtcaccggcatcccggctaccacaggagtctcttttgggacagaggtggtgtgctacgagagcccgcggccggtgctggggatccacaggctggtgttcctgctgttcgagcagctggggcggcagacggtgtacgcaccggggtggcgccagaacttcagcacccgcgacttcgccgagctctacaacctcggcctccctgtcgccgccgtctacttcaactgccagagggagtctggaaccgaaggaaaaagaatgtga

SEQ ID NO: 44 - protein, 276 aa, Oryza sativa
MTGSSCPADSSQLLYPRRGAPTTTTTSRPPLHPLIAHAPYDVVLHRSVVLSISSARDQAELASFWCYIRLGGGASAKRAAGEDDRSRSSLTAARGGGGETMSGRGRGDPLVLGRVVGDVVDPFVRRVALRVAYGAREVANGCELRPSAVADQPRVAVGGPDMRTFYTLVMVDPDAPSPSDPNLREYLHWLVTGIPATTGVSFGTEVVCYESPRPVLGIHRLVFLLFEQLGRQTVYAPGWRQNFSTRDFAELYNLGLPVAAVYFNCQRESGTEGKRM

SEQ ID NO: 45 - DNA, 630 bp, Oryza sativa
atgggccaacttcttggcaagaagtacatataccttgggctctttgacaccgaagttgaagctgcaagagcatatgacagggcagctattcgcttcaatgggagggaagctgttaccaacttcgagcctgcatcctacaatgtggatgctttaccagacgccggaaatgaggcaattgttgatggcgatcttgatttggatttgcggatttcgcaacctaatgcgcgtgactccaaaagcgatgtcgccacaactggcctccagttaacttgtgattcccctgaatcttcaaatattacagtccaccagccaatgggctcgtctccccaatggactgtgcatcaccaaagcacaccactgccccctcagcatcaacgtttgtacccatctcattgtcttggcttcctcccgaacctacaggagaggccaatggacagaaggcctgagctgggtcccatgccgttcccaacacaggcttggcaaatgcaggccccttctcacttgccattgctccacgctgcagcatcatcaggattctctgccggcgccggcgccggcgtcgccgccgccacccgccggcagccgccgttcccggcggatcaccccttctacttcccgccaaccgcctga

SEQ ID NO: 46 - protein, 209 aa, Oryza sativa
MGQLLGKKYIYLGLFDTEVEAARAYDRAAIRFNGREAVTNFEPASYNVDALPDAGNEAIVDGDLDLDLRISQPNARDSKSDVATTGLQLTCDSPESSNITVHQPMGSSPQWTVHHQSTPLPPQHQRLYPSHCLGFLPNLQERPMDRRPELGPMPFPTQAWQMQAPSHLPLLHAAASSGFSAGAGAGVAAATRRQPPFPADHPFYFPPTA

SEQ ID NO: 47 - DNA, 1110 bp, Oryza sativa
atggagaagttgctttcctcctccggcgccgccgccgccgtggcgtcgcagggccagctcccggactgcttcgtgttcccggccgaccggcgcccaccggcctccaccgcggccgtgtcgctccccgtcatcgacctctccggcccccgcgacgccgtccgccgcgccgtcctcgacgccggcaaggagctcggcttcttccaggtcagaaaacaacagaatccccaagcctgtaggtatacacacagcttctctctcaatcgcttgacatggcggtgccgtgcgtgcaaggtggtgaaccacggcgtgccgccggagacgatgcgggagatggcggcggtgtgcgaggagttcttccggctgccggcggaggacaaggcggcgttctactccgacgcggaggagaaccccaaccgcctcttctccagcaccatctacgaggtcggcgaccagcgctactggcgcgactgcctccgcctcgcctgcggcttccccgtcgccgacgacaccaacacccactggcccgacaagccccaccatctccgggatgtcacggagaagttcttcgtggcgacgaggggattggggatcgagctgctgcggctgctgtgcgaggggatggggctcaggccggactacttcgagcgcgacctcaccgccggcgatgtcatcatcaacgtcaaccactaccctccatgcccggatccgagcctgacgctgggcttgccgccgcactgcgaccgcaacctcatcaccctgctcctccagggcgacgtcttcggcctccaggtctcctacaatggcgactggatcaacgtcgaccccgtccccgacgccttcgtcgtcaactttggccacctcctcgagattgcgacgaacggagtgctgaagagcattgagcacagggcgatgacgaactcggcggtggcgaggacgtcggtggcgacgttcatgatgccgccgatggactgcctcgtcggaccggcgaaggagctcgtcggagacggcggccagccgcagtatcgcaccgtcacgttccgcgagttcatgcgcatctacaagaccgtcggcgcgcgccgcgacagcgtcgagaaggccttcaaaatctga

SEQ ID NO: 48 - protein, 369 aa, Oryza sativa
MEKLLSSSGAAAAVASQGQLPDCFVFPADRRPPASTAAVSLPVIDLSGPRDAVRRAVLDAGKELGFFQVRKQQNPQACRYTHSFSLNRLTWRCRACKVVNHGVPPETMREMAAVCEEFFRLPAEDKAAFYSDAEENPNRLFSSTIYEVGDQRYWRDCLRLACGFPVADDTNTHWPDKPHHLRDVTEKFFVATRGLGIELLRLLCEGMGLRPDYFERDLTAGDVIINVNHYPPCPDPSLTLGLPPHCDRNLITLLLQGDVFGLQVSYNGDWINVDPVPDAFVVNFGHLLEIATNGVLKSIEHRAMTNSAVARTSVATFMMPPMDCLVGPAKELVGDGGQPQYRTVTFREFMRIYKTVGARRDSVEKAFKI

SEQ ID NO: 49 - DNA, 1023 bp, Oryza sativa
atggagaagttgctttcctcctccggcgccgccgccgccgtggcgtcgcagggccagctcccggactgcttcgtgttcccggccgaccggcgcccaccggcctccaccgcggccgtgtcgctccccgtcatcgacctctccggcccccgcgacgccgtccgccgcgccgtcctcgacgccggcaaggagctcggcttcttccaggtggtgaaccacggcgtgccgccggagacgatgcgggagatggcggcggtgtgcgaggagttcttccggctgccggcggaggacaaggcggcgttctactccgacgcggaggagaaccccaaccgcctcttctccagcaccatctacgaggtcggcgaccagcgctactggcgcgactgcctccgcctcgcctgcggcttccccgtcgccgacgacaccaacacccactggcccgacaagccccaccatctccgggatgtcacggagaagttcttcgtggcgacgaggggattggggatcgagctgctgcggctgctgtgcgaggggatggggctcaggccggactacttcgagcgcgacctcaccgccggcgatgtcatcatcaacgtcaaccactaccctccatgcccggatccgagcctgacgctgggcttgccgccgcactgcgaccgcaacctcatcaccctgctcctccagggcgacgtcttcggcctccaggtctcctacaatggcgactggatcaacgtcgaccccgtccccgacgccttcgtcgtcaactttggccacctcctcgagattgcgacgaacggagtgctgaagagcatcgagcacagggcgatgacgaactcggcggtggcgaggacgtcggtggcgacgttcatgatgccgccgatggactgcctcgtcggaccggcgaaggagctcgtcggagacggcggccagccgcagtatcgcaccgtcacgttccgcgagttcatgcgcatctacaagaccgtcggcgcgcgccgcgacagcgtcgagaaggccttcaaaatctga

SEQ ID NO: 50 - protein, 340 aa, Oryza sativa
MEKLLSSSGAAAAVASQGQLPDCFVFPADRRPPASTAAVSLPVIDLSGPRDAVRRAVLDAGKELGFFQVVNHGVPPETMREMAAVCEEFFRLPAEDKAAFYSDAEENPNRLFSSTIYEVGDQRYWRDCLRLACGFPVADDTNTHWPDKPHHLRDVTEKFFVATRGLGIELLRLLCEGMGLRPDYFERDLTAGDVIINVNHYPPCPDPSLTLGLPPHCDRNLITLLLQGDVFGLQVSYNGDWINVDPVPDAFVVNFGHLLEIATNGVLKSIEHRAMTNSAVARTSVATFMMPPMDCLVGPAKELVGDGGQPQYRTVTFREFMRIYKTVGARRDSVEKAFKI

SEQ ID NO: 51 - DNA, 2580 bp, Oryza sativa
atggcggcggcgatggtggcggcggtgcatggagtggggaggcaggacaggtcgtcgcctggcggcggaggggcgccgcaagtggacacgggcaagtacgtgcgctacaccccggagcaggtggaggcgctggagcgggtctacggcgagtgccccaagcccagctcgctccgccgccagcagctcatccgcgagtgccccatactcagcaacatcgagcccaagcagatcaaggtctggttccagaaccgcaggtgccgcgagaagcagcgcaaggaggcatctcgcctgcaaactgtgaaccggaagttgactgcgatgaacaagctgttgatggaggagaatgacaggctgcagaagcaggtgtcccgcctcgtttacgagaacgggtacatgcggcagcagctccataatccttctgtggcaactacagacacgagctgtgagtctgtggttacaagtggtcaacaccaccaacagcaaaacccagcagccacgcgtccgcaaagggacgcgaacaacccagctggtctactcgctatagctgaggagaccttggcagagttcctgtcgaaagcgacgggtactgctgtcgattgggtgcaaatggttgggatgaagcctggtccggattccattggaatcatcgctgtttcgcacaattgtagtggcgtagcagcacgagcttgcggccttgtgagccttgagcccacaaaggttgctgagatccttaaggatcgcccatcttggtatcgcgattgccggtgtgtggatgtgctccatgttatccctacgggtaatggtggaactattgagcttatctacatgcagacttatgcaccgacaactttggcggcaccacgtgacttctggatactccgttacactagtggtcttgaggatggaagtcttgtgatctgtgagagatcattgactcaatccactggtggcccatcaggacctaacactccaaactttgtcagagccgaggtgcttcctagcggctatttgatccgcccatgtgaggggggtggttccatgattcacattgtggatcatgttgatttggatgcttggagtgtgcctgaggtccttagaccactttatgaatctcctaagatccttgcgcagaagatgacaattgctgcactgcgtcacattaggcaaattgcacatgaatcaagtggggaaatgccctatggagggggccgtcagccagcagttctgagaacatttagtcagaggctaagcagaggcttcaatgatgctgtcaatggattcccggatgatggctggtcactgatgagcagcgatggtgctgaggatgtgacaattgcttttaactcatctccaaacaagcttgttggatctcatgtcaactcctcccagctgttttctgcaattggaggcggcatcttgtgtgcaaaggcatctatgctgttacagaatgtaccacctgctctcctagtgcgatttttgagggagcaccgctctgaatgggctgatcctggtgttgatgcttattctgctgctgcgttgagggctagtccatatgcagttcctggtctgcgagctggtggttttatgggcagtcaggttatattgccacttgcgcacaccttagagcatgaagagttcctggaggtcattaggcttgagggacacagcctctgccatgatgaggtcgttctatcaagagatatgtaccttctgcagctgtgcagcggtgtagatgaaaatgctgcgggtgcatgtgcccagcttgtctttgcacccattgacgaatcttttgctgatgatgcaccactgctaccctcaggcttccgtgtgataccactggatggcaagacggatgcaccatctgcgacacgcacacttgacctggcatctactcttgaggttggatcgggtgggactacacgggcttctagtgacacctcgagcacctgcaacacaagatcggtcctgaccatagctttccaattctcatatgaaaaccaccttcgtgaaagcgtagcggcaatggccaggcagtatgtccggaccgtggtggcatcagtgcagagagtggccatggcaatagctccttcacgtcttggtggacagattgaaacaaagaaccctccaggatctcctgaggcccatacgcttgcaaggtggattgggaggagctaccgattccacactggagcggatccccttcgcacagactcacaaagcacggattcttccttgaaagcaatgtggcaacactctgattcaatcatgtgctgctccctgaaggctgctcctgtgttcaccttcgccaaccaagccggcctcgacatgctggagacgacgctgattgctctccaggacatctcgctcgagaagatccttgatgatgatggccggaaggcactatgtacagagttccccaagatcatgcagcagggtttcgcctacctcccgggcggcgtgtgcgtgtcgagcatggggcggccggtgtcgtacgagcaggcggtggcgtggaaggtcctgagcgacgacgacacgccgcactgcctcgccttcatgttcgtcaactggtcattcgtctga

SEQ ID NO: 52 - protein, 859 aa, Oryza sativa
MAAAMVAAVHGVGRQDRSSPGGGGAPQVDTGKYVRYTPEQVEALERVYGECPKPSSLRRQQLIRECPILSNIEPKQIKVWFQNRRCREKQRKEASRLQTVNRKLTAMNKLLMEENDRLQKQVSRLVYENGYMRQQLHNPSVATTDTSCESVVTSGQHHQQQNPAATRPQRDANNPAGLLAIAEETLAEFLSKATGTAVDWVQMVGMKPGPDSIGIIAVSHNCSGVAARACGLVSLEPTKVAEILKDRPSWYRDCRCVDVLHVIPTGNGGTIELIYMQTYAPTTLAAPRDFWILRYTSGLEDGSLVICERSLTQSTGGPSGPNTPNFVRAEVLPSGYLIRPCEGGGSMIHIVDHVDLDAWSVPEVLRPLYESPKILAQKMTIAALRHIRQIAHESSGEMPYGGGRQPAVLRTFSQRLSRGFNDAVNGFPDDGWSLMSSDGAEDVTIAFNSSPNKLVGSHVNSSQLFSAIGGGILCAKASMLLQNVPPALLVRFLREHRSEWADPGVDAYSAAALRASPYAVPGLRAGGFMGSQVILPLAHTLEHEEFLEVIRLEGHSLCHDEVVLSRDMYLLQLCSGVDENAAGACAQLVFAPIDESFADDAPLLPSGFRVIPLDGKTDAPSATRTLDLASTLEVGSGGTTRASSDTSSTCNTRSVLTIAFQFSYENHLRESVAAMARQYVRTVVASVQRVAMAIAPSRLGGQIETKNPPGSPEAHTLARWIGRSYRFHTGADPLRTDSQSTDSSLKAMWQHSDSIMCCSLKAAPVFTFANQAGLDMLETTLIALQDISLEKILDDDGRKALCTEFPKIMQQGFAYLPGGVCVSSMGRPVSYEQAVAWKVLSDDDTPHCLAFMFVNWSFV

SEQ ID NO: 53 - DNA, 1113 bp, Oryza sativa
atgatgatgtcttcttcgcaaacaccagtacggatcgccttcgttttcctcgtcatcctcgccgcgactgatgcgcacagcgaccaccgaactccgccgccggcgtgcggaggcgcggccgtgggaggggaatgccacagcgtggccagggcgctccgcctgaagctgatcgccatcccggcgatcctcgccgccagcgtggccggcgtgtgcctgccgatcttcgcccggtccgtgccggcgctccgccccgacggcggcctcttcgccgtcgtgaaggcgttcgcgtcgggcgtcatcctcggcaccggctacatgcacgtgctcccggactcgttcaacgacctcacctcgccgtgcctgcccaggaagccatggccggagttcccgttcgcggcgttcgtcgccatgctcgccgccgtgttcacgctcatggtggactcgctcatgctcacgttccacacgcggggcagcaagggacgggccagcagcgccgtcgcgcaccacggcgaccacgggcactgtcacgctcacgcgctggggcaagcagacgtcgctgcgctgtcgacgacggaggcggcggatcagggcagcggcgacgtcgaggccggtaacaccaccaaggcgcagcttctcaggaatcgcgtcattgtgcaggttctcgagatgggcatcgtggtgcactcagtggtgatcgggctgggcatgggggcgtcgcagaacgtgtgcacgatccggccgctggtggcggcgctgtgcttccaccagatgttcgaggggatggggctcggcggctgcatcctgcaggcggggtacggcgggaggacgaggtcggcgctggtcttcttcttctccaccacgacgccgttcgggatcgcgctggggctcgcgctgaccagggtgtacagcgacagcagcccgacggcgctggtcgtcgtcggcctgctcaacgcggcgtcggcggggctgctgcactacatggcgctggtggagctcctcgccgccgatttcatggggcccaagctgcagggcaacgtccgtctccagctcgccgcgtccctcgccatcctcctcggcgccggcggcatgtccgtcatggccaagtgggcgtga

SEQ ID NO: 54 - protein, 370 aa, Oryza sativa
MMMSSSQTPVRIAFVFLVILAATDAHSDHRTPPPACGGAAVGGECHSVARALRLKLIAIPAILAASVAGVCLPIFARSVPALRPDGGLFAVVKAFASGVILGTGYMHVLPDSFNDLTSPCLPRKPWPEFPFAAFVAMLAAVFTLMVDSLMLTFHTRGSKGRASSAVAHHGDHGHCHAHALGQADVAALSTTEAADQGSGDVEAGNTTKAQLLRNRVIVQVLEMGIVVHSVVIGLGMGASQNVCTIRPLVAALCFHQMFEGMGLGGCILQAGYGGRTRSALVFFFSTTTPFGIALGLALTRVYSDSSPTALVVVGLLNAASAGLLHYMALVELLAADFMGPKLQGNVRLQLAASLAILLGAGGMSVMAKWA

SEQ ID NO: 55 - DNA, 1200 bp, Oryza sativa
atggcggcgggcggcggcggagatggtggtggtggtggtgggcgggcggtgatcccgatgggacacggcgacccgtcggtgttcccgtgcttccggacgacggcggacgccgtggacgccgtggcggcggcgctccggtccggggagcacaactcctactcctcctgcgtcggcctcgagcctgcacggaggtctatcgcgcggtacttatcgcgagacttgccatatgagctatcagctgatgatgtgtacctgacaagtggctgtgctcaagcgattgagatcatctgctctgtcctagctcgccctggtgccaacatcctgtgcccaaggccagggtacctgttccacgaggcacgcgcagtgttcaatggcatggaggtcaggtactttgatcttctcccagagagtggctgggaggttgatcttgatggagtgcaggaacttgctgacaagaacacggttgcaatggtcattatcaatccaggaaatccctgtggcaatgtctacacttctgagcatttggccaaggttgccgagaccgcgaaaaagcttggcatctttgtcattgcagatgaagtgtatgcacatttgacgtttgggcagaataaatttgtgccgatgggggtgtttggctcagtagctccagttcttacgttaggatcgatatcgaagagatgggttgtgcctggatggcgacttggatggattgtgacgagcgatcctaatggcgtatttcaaaggaccaaggtagtggaaagcatccagagttaccttgatatctcggctgatcctgcaacatttatccagggagcaattccacaactcatagagaacacaaaggaagaattcttcgaaaagaccgtcgatgtcctaaggcagaccgcagatatttgttgggagaagctgaagggcatcagttgcatcacttgcccaagcaaacctgagggttccatgtttgtcatggtgaaactggatctgtcctgcctgcaaggcatcaaagatgacatggacttctgctgccagctagcaaaggaagaattggtgattcttttgccaggatgtgctgttggatacaagaattggctccgaatcaccttcgccatcgaaccgtcttctcttgaagatggtattgataggctcaagtccttctgctcgcgacatagcaagccgaaagttcaccggtctcttgaaacatga

SEQ ID NO: 56 - protein, 399 aa, Oryza sativa
MAAGGGGDGGGGGGRAVIPMGHGDPSVFPCFRTTADAVDAVAAALRSGEHNSYSSCVGLEPARRSIARYLSRDLPYELSADDVYLTSGCAQAIEIICSVLARPGANILCPRPGYLFHEARAVFNGMEVRYFDLLPESGWEVDLDGVQELADKNTVAMVIINPGNPCGNVYTSEHLAKVAETAKKLGIFVIADEVYAHLTFGQNKFVPMGVFGSVAPVLTLGSISKRWVVPGWRLGWIVTSDPNGVFQRTKVVESIQSYLDISADPATFIQGAIPQLIENTKEEFFEKTVDVLRQTADICWEKLKGISCITCPSKPEGSMFVMVKLDLSCLQGIKDDMDFCCQLAKEELVILLPGCAVGYKNWLRITFAIEPSSLEDGIDRLKSFCSRHSKPKVHRSLET

SEQ ID NO: 57 - DNA, 999 bp, Oryza sativa
atggaggctcagaaccaagaggtcgctgccctggtcgagaagatcgccggcctccacgccgccatctccaagctgccgtcgctgagcccatccgccgaggtggacgcgctcttcaccgacctcgtcacggcgtgcgtcccggcgagccccgtcgacgtggccaagctcggcccggaggcgcaggcgatgcgggaggagctcatccgcctctgctccgccgccgagggccacctcgaggcgcactacgccgacatgctcgccgccttcgacaacccgctcgaccacctcgcccgcttcccgtactacggcaactacgtcaacctgagcaagctggagtacgacctcctcgtccgctacgtccccggcattgcccccacccgcgtcgccttcgtcgggtcgggcccgctgccgttcagctccctcgtgctcgccgcgcaccacctgccggacgcggtgttcgacaactacgaccggtgcggcgcggccaacgagcgggcgaggaggctgttccgcggcgccgacgagggcctcggcgcgcgcatggcgttccacaccgccgacgtggcgaccctgacgggggagctcggcgcgtacgacgtcgtgttcctggcggcgctcgtgggcatggcggccgaggagaaggccggggtgatcgcgcacctgggcgcgcacatggcggacggcgcggcgctcgtcgtgcggagcgcgcacggggcgcgcgggttcctgtacccgatcgtcgatcccgaggacgtcaggcgtggcgggttcgacgttctggcggtgtgccacccggaggacgaggtgatcaactccgtcatcgtcgcccgcaaggtcggtgccgccgccgccgccgccgcggcgcgcagagacgagctcgcggactcgcgcggcgtggttctgccggtggtcgggccgccgtccacgtgctgcaaggtggaggcgagcgcggttgagaaggcagaagagtttgccgccaacaaggagctgtccgtctaa

SEQ ID NO: 58 - protein, 332 aa, Oryza sativa
MEAQNQEVAALVEKIAGLHAAISKLPSLSPSAEVDALFTDLVTACVPASPVDVAKLGPEAQAMREELIRLCSAAEGHLEAHYADMLAAFDNPLDHLARFPYYGNYVNLSKLEYDLLVRYVPGIAPTRVAFVGSGPLPFSSLVLAAHHLPDAVFDNYDRCGAANERARRLFRGADEGLGARMAFHTADVATLTGELGAYDVVFLAALVGMAAEEKAGVIAHLGAHMADGAALVVRSAHGARGFLYPIVDPEDVRRGGFDVLAVCHPEDEVINSVIVARKVGAAAAAAAARRDELADSRGVVLPVVGPPSTCCKVEASAVEKAEEFAANKELSV

SEQ ID NO: 59 - DNA, 1557 bp, Oryza sativa
atgggggtgacgaaggcggaggcggttgccggcgacggcgggaaggtggtggacgacatcgaggcgctcgccgatctcagaaaggagccagcatggaaaaggtttttgtcccacattggaccaggctttatggtctgtttggcttacctcgatcctggaaatatggagactgatctacaagctggtgccaatcacaaatatgagcttctttgggtgattttgattggcctgatctttgcactgattatacaatcattatcggctaatcttggagtggtgacagggcgccatcttgctgagctgtgcaagactgaatatccagtatgggtgaaaacctgcttatggctgctagcagaactagctgtgattgcttccgatattccagaagttataggtacaggatttgctttcaaccttttgttccacataccagtgtggaccggggttctcattgctggctctagcacgcttcttcttcttggactgcaaagatatggggtaaggaaactggaggttgtggtcgcgctattggtgtttgtcatggcagggtgcttcttcgtggagatgagcatagtcaagcctccggttaatgaggtccttcaaggattattcatccccaggctcagtgggcccggtgccacaggagactccattgcccttcttggggctcttgtaatgccgcacaatctattcttgcactctgccctggtgctgtcgaggaatacacccgcatcagcaaaaggaatgaaggatgtctgtaggttcttcctctttgagagtgggatagctcttttcgtggctctgcttgtcaacattgcaatcatctctgtctccggcactgtatgtaatgcaaccaacctttcaccagaagatgctgtaaaatgcagcgaccttacattggactcctcatccttcctcctcaggaatgtgctaggaaagtcaagtgcgacagtgtacggtgtagcactcttggcttctggacagagctcgaccattactggcacttatgctgggcaatatgttatgcaggggttcctagacatcaaaatgaaacagtggcttcggaacctgatgacacgcagcattgccattgtgcctagcttaatcgtctccatcattggagggtctagtggcgccggtcgtctcatcgtcattgcatcgatgattctatcctttgagctaccatttgctctgatccctcttctcaagttcagcagcagcagcaacaagatgggtgaaaacaagaactccatctatattgttggattctcctgggtgctggggttcgtcatcatcgggataaacatctacttcctcagcacaaagctggtcggctggatcctccacaacgcgctcccgacattcgccaacgtgctcatcggcatcgtgctgttcccgctcatgctgctctacgtcgtcgccgtgatctacctgacgttcaggaaggacaccgtcaagttcgtgtctcgtcgtgagctgcaggccggcgacgacaccgagaaagcacaggtagccacctgcgtcgccgacgagcacagcaaagagccacccgtgtag

SEQ ID NO: 60 - protein, 518 aa, Oryza sativa
MGVTKAEAVAGDGGKVVDDIEALADLRKEPAWKRFLSHIGPGFMVCLAYLDPGNMETDLQAGANHKYELLWVILIGLIFALIIQSLSANLGVVTGRHLAELCKTEYPVWVKTCLWLLAELAVIASDIPEVIGTGFAFNLLFHIPVWTGVLIAGSSTLLLLGLQRYGVRKLEVVVALLVFVMAGCFFVEMSIVKPPVNEVLQGLFIPRLSGPGATGDSIALLGALVMPHNLFLHSALVLSRNTPASAKGMKDVCRFFLFESGIALFVALLVNIAIISVSGTVCNATNLSPEDAVKCSDLTLDSSSFLLRNVLGKSSATVYGVALLASGQSSTITGTYAGQYVMQGFLDIKMKQWLRNLMTRSIAIVPSLIVSIIGGSSGAGRLIVIASMILSFELPFALIPLLKFSSSSNKMGENKNSIYIVGFSWVLGFVIIGINIYFLSTKLVGWILHNALPTFANVLIGIVLFPLMLLYVVAVIYLTFRKDTVKFVSRRELQAGDDTEKAQVATCVADEHSKEPPV

SEQ ID NO: 61 - DNA, 1395 bp, Oryza sativa
atgcgcccggccttctcgtggcgcaagctgtggcggttcacggggcccgggttcctcatgtgcatcgcgttcctcgacccggggaacctggagggcgacctgcaggccggcgcggcggcggggtaccagctgctgtggctgctgctgtgggcgacggtcatgggcgccctggtgcagctgctctccgcgcggctcggggtcgccacggggaagcacctcgccgagctctgcagggaggagtacccgccctgggccacgcgcgcgctctgggccatgaccgagctcgcgctcgtcggcgcggacatccaggaggtgattggcagcgcgattgccatcaagatcctctccgctggcaccgtcccgctctggggcggcgtcgtcatcaccgcgttcgattgcttcatctttttattcctggagaactatggagtgagaaaattggaagcatttttcggagtcctgattgcagtcatggcagtatcatttgcaattatgtttggtgaaacaaagccaagtggcaaggagcttctgattggtttggtggttccaaagttgagttcaaggacaatcaaacaagcagttggaattgtgggctgcataatcatgccccacaatgtcttcttgcactcagcactagtgcagtcaaggaagattgacacaaacaagaaatcccgtgttcaagaagcagtgttctattacaacattgagtccattcttgccctcattgtctcgttctttattaacatctgtgtcacaacagtttttgcgaaaggattttatggatctgaacaagctgatggtataggtcttgagaatgctggacagtacttacagcagaaatatgggactgcattctttcctatactgtatatctgggctattgggctgttagcatctggacagagtagcactattactggcacatatgcaggccaatttgttatgggaggcttccttaatcttcggttgaagaagtggttaagagcaatgattactcgaagctttgcaattattccaactatgattgtggctttattttttgacacggaggatcctacaatggacattctgaatgaggcactcaatgttcttcaatccatacagataccatttgcactgattcctctcatcacacttgtctcaaaggagcaagtcatgggatcatttgtggttggtcctatcacaaaagtgattagctggattgttacagtattcttgatgctcatcaatgggtatcttatactgtccttctatgccactgaagtccggggagcattggttcggtcaagcttgtgcgttgtattggcagtttaccttgcattcatcgtctatcttatcatgcgaaatacctcactgtattctcgcctccgctcagcaatgacaaagagcacatga

SEQ ID NO: 62 - protein, 464 aa, Oryza sativa
MRPAFSWRKLWRFTGPGFLMCIAFLDPGNLEGDLQAGAAAGYQLLWLLLWATVMGALVQLLSARLGVATGKHLAELCREEYPPWATRALWAMTELALVGADIQEVIGSAIAIKILSAGTVPLWGGVVITAFDCFIFLFLENYGVRKLEAFFGVLIAVMAVSFAIMFGETKPSGKELLIGLVVPKLSSRTIKQAVGIVGCIIMPHNVFLHSALVQSRKIDTNKKSRVQEAVFYYNIESILALIVSFFINICVTTVFAKGFYGSEQADGIGLENAGQYLQQKYGTAFFPILYIWAIGLLASGQSSTITGTYAGQFVMGGFLNLRLKKWLRAMITRSFAIIPTMIVALFFDTEDPTMDILNEALNVLQSIQIPFALIPLITLVSKEQVMGSFVVGPITKVISWIVTVFLMLINGYLILSFYATEVRGALVRSSLCVVLAVYLAFIVYLIMRNTSLYSRLRSAMTKST

SEQ ID NO: 63 - DNA, 1620 bp, Oryza sativa
atggcgcggcaggagcagcagcagcacctgcaggtgctgagcgcgctggacgcggcgaagacgcagtggtaccacttcacggcgatcgtcgtcgccggcatgggcttcttcaccgacgcctacgacctcttctgcatctccctcgtcaccaagctgctcggccgcatctactacaccgacctcgccaaggagaaccccggcagcctgccgcccaacgtcgccgcggcggtgaacggagtcgcgttctgcggcacgctggcggggcagctcttcttcgggtggctcggcgacaagctcggccggaagagcgtgtacgggatgacgctgctgatgatggtcatctgctccatcgcgtcggggctctcgttctcgcacacgcccaccagcgtcatggcgacgctctgcttcttccggttctggctcggattcggcatcggcggcgactacccgctgtcggcgacgatcatgtcggagtacgccaacaagaagacccgcggcgcgttcatcgccgccgtgttcgcgatgcaggggttcggcatcctcgccggcggcatcgtcaccctcatcatctcctccgcgttccgcgccgggttcccggcgccggcgtaccaggacgaccgcgcgggctccaccgtccgccaggccgactacgtgtggcggatcatcctcatgctcggcgccatgccggcgctgctcacctactactggcggatgaagatgccggagacggcgcgctacaccgccctcgtcgccaagaacgccaagcaggccgccgccgacatgtccaaggtgctccaggtcgagatccaggaggagcaggacaagctggagcagatggtgacccggaacagcagcagcttcggcctcttctcccgccagttcgcgcgccgccacggcctccacctcgtcggcaccgccacgacatggttcctcctcgacatcgccttctacagccagaacctgttccagaaggacatcttcaccagcatcaactggatccccaaggccaagaccatgtcggcgctggaggaggtgttccgcatcgcgcgcgcccagacgctcatcgccctgtgcggcaccgtcccgggctactggttcaccgtcttcctcatcgacatcgtcggccgcttcgccatccagctgctagggtttttcatgatgaccgtgttcatgctcggcctcgccgtgccgtaccaccactggacgacgaaggggaaccacatcggcttcgtcgtcatgtacgccttcaccttcttcttcgccaacttcggccccaactccaccaccttcatcgtgccggcggagatcttcccggcgaggctgcgttccacctgccacggcatctcggcggcggcggggaaggccggcgccatcatcggatcgttcgggttcctgtacgcggcgcaggacccgcacaagcccgacgccgggtacaaacccgggatcggggtgaggaactcgctgttcgtgctcgccggatgcaacctgctcgggttcatctgcacgttcctcgtgccggagtcgaaggggaagtcgctggaggagatgtccggcgaggcggaggacgacgacgacgaggtggccgccgccggcggtggcgccgccgtgcggccgctctag

SEQ ID NO: 64 - protein, 539 aa, Oryza sativa
MARQEQQQHLQVLSALDAAKTQWYHFTAIVVAGMGFFTDAYDLFCISLVTKLLGRIYYTDLAKENPGSLPPNVAAAVNGVAFCGTLAGQLFFGWLGDKLGRKSVYGMTLLMMVICSIASGLSFSHTPTSVMATLCFFRFWLGFGIGGDYPLSATIMSEYANKKTRGAFIAAVFAMQGFGILAGGIVTLIISSAFRAGFPAPAYQDDRAGSTVRQADYVWRIILMLGAMPALLTYYWRMKMPETARYTALVAKNAKQAAADMSKVLQVEIQEEQDKLEQMVTRNSSSFGLFSRQFARRHGLHLVGTATTWFLLDIAFYSQNLFQKDIFTSINWIPKAKTMSALEEVFRIARAQTLIALCGTVPGYWFTVFLIDIVGRFAIQLLGFFMMTVFMLGLAVPYHHWTTKGNHIGFVVMYAFTFFFANFGPNSTTFIVPAEIFPARLRSTCHGISAAAGKAGAIIGSFGFLYAAQDPHKPDAGYKPGIGVRNSLFVLAGCNLLGFICTFLVPESKGKSLEEMSGEAEDDDDEVAAAGGGAAVRPL

(19) United States
(12) Patent Application Publication
(10) Pub. No.: US 2004/0219675 A1
(43) Pub. Date: Nov. 4, 2004

(54) Nucleic acid molecules from rice encoding proteins for abiotic stress tolerance, enhanced yeild, disease resistance and altered nutritional quality and uses thereof

(76) Inventors: Manuel B Sainz, Durham, NC (US); John Salmeron, Hillsborough, NC (US); Laura Weislo, Raleigh, NC (US)

Correspondence Address: Mary Kakefuda, Syngenta Biotechnology, 3054 Cornwallis Road, Research Triangle Park, NC, 27709, US

(21) Appl. No.: 10/491,733

(22) PCT Filed: Nov. 27, 2002
(86) PCT No.: PCT/US02/38359

Related U.S. Application Data
(60) Provisional application No. 60/334,501, filed on Nov. 30, 2001.

(51) Int. Cl. C12N 15/82; C07H 21/04; C12N 15/29; A01H 5/00; C12N 15/09; C07K 1/00; C12N 5/04; C12N 5/10; C12N 5/14; C07K 14/00; C12N 15/63; C12N 15/00; C12N 15/87; A61K 39/395

(52) U.S. Cl. 435/468; 536/23.6; 435/320.1; 435/419; 800/301; 800/320.2; 800/320; 800/278; 800/284; 530/350; 435/69.1; 800/285; 800/286; 424/130.1; 800/287; 800/295

(57) ABSTRACT

The present invention encompasses nucleic acid molecules isolated from Oryza sativa that encode proteins for conferring abiotic stress tolerance, enhanced yield, disease resistance, or altered nutritional composition in plants. The invention further relates to expression of these molecules in microorganisms and transgenic plants for altering these characteristics of the organism.

This application claims the benefit of U.S. Provisional Application No. 60/334,501 filed Nov. 30, 2001, which is incorporated by reference herein in its entirety.

### FIELD OF THE INVENTION

The present invention pertains to nucleic acid molecules isolated from Oryza sativa comprising nucleotide sequences that encode proteins for abiotic stress tolerance, enhanced yield, disease resistance or altered nutritional quality. The invention particularly relates to methods of using nucleic acid molecules and/or proteins from Rice in transgenic plants to conferthe above-identified agronomic traits.

### BACKGROUND OF THE INVENTION

Improvement of the agronomic characteristics of crop plants has been ongoing since the beginning of agriculture. Most of the land suitable for crop production is currently being used. As human populations continue to increase, improved crop varieties will be required to adequately provide our food and feed (Trewavas (2001) Plant Physiol. 125: 174-179). To avoid catastrophic famines and malnutrition, future crop cultivars will need to have improved yields with equivalent farm inputs. These cultivars will need to more effectively withstand adverse conditions such as drought, soil salinity or disease, which will be especially important as marginal lands are brought into cultivation. Finally, we will need cultivars with altered nutrient composition to enhance human and animal nutrition, and to enable more efficient food and feed processing, by designing cultivars for specific end-uses.

Recent scientific advances have together identified many candidate genes associated with traits of agronomic interest. Scientific approaches used to identify genes involved in a pathway or response of interest associated with an agronomic trait include genetics, genomics, bioinformatics, and functional genomics. Genetics is the scientific study of the mechanisms of inheritance. By identifying mutations which alter the pathway or response of interest, classical (or forward) genetics can help to identify the genes involved in these pathways or responses. For example, a mutant with enhanced susceptibility to disease may identify an important component of the plant signal transduction pathway leading from pathogen recognition to disease resistance. Genomics is the system-level study of an organism's genome, including genes and corresponding gene products—RNA and proteins. Genomic approaches have provided sequence information from diverse plant species, including full-length and partial cDNA sequences; more recently the complete genomic sequence of Arabidopsis thaliana became available. A unique proprietary resource used for purposes of this invention is the Syngenta draft genomic sequence of rice (Oryza sativa). As part of genomics, bioinformatics approaches process raw sequence information and can be used, for example, to help identify genes in a genomic sequence.

Functional genomics is the assignment of function to genes and their products. Functional genomics makes use of a variety of approaches to identify genes in a particular pathway or response of interest. The use of genetics to help assign function is described in the paragraph above. Using, for example, similarity searches, alignments and phylogenetic analyses, bioinformatics can often identify homologs of a gene product of interest. Very similar homologs (eg. >˜90% amino acid identity over the entire length of the protein) are very likely orthologs, i.e. share the same function in different organisms. Thus bioinformatics is another approach to assigning function to genes identified through genomics.

Functional genomics can make use of additional approaches. Expression analysis uses high density DNA microarrays to monitor the mRNA expression of thousands of genes in a single experiment. Experimental treatments can include those eliciting a response of interest, such as the disease resistance response in plants infected with a pathogen. To give additional examples of the use of microarrays, mRNA expression levels can be monitored in distinct tissues over a developmental time course, or in mutants affected in a response of interest. Proteomics can also help to assign function, by assaying the expression and post-translational modifications of hundreds of proteins in a single experiment. Proteomics approaches are in many cases analogous to the approaches taken for monitoring mRNA expression in microarray experiments. Protein-protein interactions can also help to assign proteins to a given pathway or response, by identifying proteins which interact with known components of the pathway or response. For functional genomics, protein-protein interactions are often studied using large-scale yeast two-hybrid assays. Another approach to assigning gene function is to express the corresponding protein in a heterologous host, for example the bacterium Escherichia coli, followed by purification and enzymatic assays.

The generation and analysis of plants transgenic for a gene of interest can be used for plant functional genomics, with several advantages. The gene can often be both overexpressed and underexpressed (“knocked out”), thereby increasing the chances of observing a phenotype linking the gene to a pathway or response of interest. Two aspects of transgenic functional genomics help lend a high level of confidence to functional assignment by this approach. First, phenotypic observations are carried out in the context of the living plant. Second, the range of phenotypes observed often correlates well with observed expression levels.

Transgenic functional genomics is especially valuable in improved cultivar development. Only genes that function in a pathway or response of interest, and that in addition are able to confer a desired trait-based phenotype, are promoted to candidate genes for crop improvement efforts. Such efforts can take various forms, for example the generation of transgenic crops or marker-assisted breeding using desirable alleles of the gene.

### SUMMARY OF THE INVENTION

This Summary of Invention lists several embodiments of the invention, and in many cases lists variations and permutations of these embodiments. This Summary is merely exemplary of the numerous and varied embodiments. Mention of one or more preferred features of a given embodiment is likewise exemplary. Such embodiment can typically exist with or without the feature(s) mentioned; likewise, those features can be applied to other embodiments of the invention, whether listed in this Summary or not. To avoid excessive repetition, this Summary does not list or suggest all possible combinations of such features.

Embodiments of the present invention provide nucleotide and amino acid sequences known as cDNAs from rice.

Embodiments of the present invention relate to an isolated nucleic acid comprising or consisting of a nucleotide sequence including:

(a) a nucleotide sequence listed in odd numbered sequences of SEQ ID NO:1-63, fragment, domain, or feature thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c).

In a preferred embodiment, the substantial similarity is at least about 65% identity, preferably about 80% identity, preferably 90%, and more preferably at least about 95% identity to the nucleotide sequence listed in odd numbered sequences of SEQ ID Nos:1-63, fragment, domain, or feature thereof.

In a preferred embodiment, the sequence having substantial similarity to the nucleotide sequence listed in odd numbered sequences of SEQ ID Nos:1-63, fragment, domain, or feature thereof, is from a plant. In a preferred embodiment, the plant is a dicot. In another preferred embodiment, the plant is a gymnosperm. In a more preferred embodiment, the plant is a monocot. In another preferred embodiment, the plant is rice, wheat, barley, rye, corn, potato, canola, soybean, sunflower, carrot, sweet potato, sugarbeet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum or sugarcane. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum sp., or teosinte. In a most preferred embodiment, the cereal is rice.

In a preferred embodiment, the nucleic acid is expressed in a specific location or tissue of a plant. In a more preferred embodiment, the location or tissue is for example, but not limited to, epidermis, vascular tissue, meristem, cambium, cortex, pith, leaf, and flower. In a most preferred embodiment, the location or tissue is a leaf, sheath, flower, root or seed. In another preferred embodiment, the nucleic acid encodes a polypeptide involved in a function such as, for example, but not limited to, carbon metabolism, photosynthesis, signal transduction, cell growth, reproduction, disease processes, gene regulation, and differentiation. In a more preferred embodiment, the nucleic acid encodes a polypeptide involved in abiotic stress tolerance, enhanced yield, disease resistance, or nutritional content.

In a preferred embodiment, the isolated nucleic acid comprising or consisting of a nucleotide sequence capable of hybridizing to a nucleotide sequence listed in odd numbered sequences of SEQ ID Nos:1-63, or fragment, domain, or feature thereof. In a preferred embodiment, hybridization allows the sequence to form a duplex atmedium or high stringency. Embodiments of the present invention also encompass a nucleotide sequence complementary to a nucleotide sequence listed in odd numbered sequences of SEQ ID Nos:1-63, or fragment, domain, or feature thereof. Embodiments of the present invention further encompass a nucleotide sequence complementary to a nucleotide sequence that has substantial similarity or is capable of hybridizing to a nucleotide sequence listed in odd numbered sequences of SEQ ID Nos:1-63, or fragment, domain, or feature thereof.

In a preferred embodiment, the nucleotide sequence having substantial similarity is an allelic variant of the nucleotide sequence listed in odd numbered sequences of SEQ ID Nos:1-63, or fragment, domain, or feature thereof. In an alternate embodiment, the sequence having substantial similarity is a naturally occurring variant. In another alternate embodiment, the sequence having substantial similarity is a polymorphic variant of the nucleotide sequence listed in odd numbered sequences of SEQ ID Nos:1-63, or fragment, domain, or feature thereof.

In a preferred embodiment, the isolated nucleic acid contains a plurality of regions having the nucleotide sequence listed in odd numbered sequences of SEQ ID Nos:1-63, or exon, domain, or feature thereof.

In a preferred embodiment, the isolated nucleic acid contains a polypeptide-encoding sequence. In a more preferred embodiment, the polypeptide-encoding sequence contains a 20 base pair nucleotide portion identical in sequence to a consecutive 20 base pair nucleotide portion of a nucleic acid sequence listed in odd numbered sequences of SEQ ID Nos:1-63. In a more preferred embodiment, the polypeptide contains a polypeptide sequence listed in even numbered sequences of SEQ ID Nos:2-64, or a fragment thereof. In a more preferred embodiment, a polypeptide described in Tables 1-4. In a more preferred embodiment, the polypeptide is a plant polypeptide. In a more preferred embodiment, the plant is a dicot. In a more preferred embodiment, the plant is a gymnosperm. In a more preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, miloflax, gramma grass, Tripsacum, and teosinte. In a most preferred embodiment, the cereal is rice.

In one embodiment, the polypeptide is expressed throughout the plant. In a more preferred embodiment, the polypeptide is expressed in a specific location or tissue of a plant. In a more preferred embodiment, the location or tissue may be, for example, epidermis, root, vascular tissue, meristem, cambium, cortex, pith, leaf, and flower. In a most preferred embodiment, the location or tissue is a seed.

In a preferred embodiment, the polypeptide is involved in a function such as abiotic stress tolerance, enhanced yield, disease resistance or nutritional content.

In a preferred embodiment, the sequence of the isolated nucleic acid encodes a polypeptide useful for generating an antibody having immunoreactivity against a polypeptide encoded by a nucleotide sequence listed in even numbered sequences of SEQ ID Nos:2-64, or fragment, domain, or feature thereof.

In a preferred embodiment, the sequence having substantial similarity contains a deletion or insertion of at least one nucleotide. In a more preferred embodiment, the deletion or insertion is of less than about thirty nucleotides. In a most preferred embodiment, the deletion or insertion is of less than about five nucleotides.

In a preferred embodiment, the sequence of the isolated nucleic acid having substantial similarity comprises or consists of a substitution in at least one codon. In a preferred embodiment, the substitution is conservative.

Embodiments of the present invention also relate to the an isolated nucleic acid molecule comprising or consisting of a nucleotide sequence, its complement, or its reverse complement, encoding a polypeptide including:

(a) a polypeptide sequence listed in even numbered sequences of SEQ ID Nos:2-64, or a fragment, domain, repeat, feature, or chimera thereof;

(b) a polypeptide sequence having substantial similarity to (a);

(c) a polypeptide sequence encoded by a nucleotide sequence identical to or having substantial similarity to a nucleotide sequence listed in odd numbered sequences of SEQ ID Nos:1-63, or a fragment, domain, or feature thereof, or a sequence complementary thereto;

(d) a polypeptide sequence encoded by a nucleotide sequence capable of hybridizing under medium stringency conditions to a nucleotide sequence listed in odd numbered sequences of SEQ ID Nos:1-63, or to a sequence complementary thereto; and

(e) a functional fragment of (a), (b), (c) or (d).

In another preferred embodiment, the polypeptide having substantial similarity is an allelic variant of a polypeptide sequence listed in even numbered sequences of SEQ ID NOS:2-64, or a fragment, domain, repeat, feature, or chimeras thereof. In another preferred embodiment, the isolated nucleic acid includes a plurality of regions from the polypeptide sequence encoded by a nucleotide sequence identical to or having substantial similarity to a nucleotide sequence listed in odd numbered sequences of SEQ ID NOS:1-63, or fragment, domain, or feature thereof, or a sequence complementary thereto.

In another preferred embodiment, the polypeptide is a polypeptide sequence listed in even numbered sequences of SEQ ID NOS:2-64. In another preferred embodiment, the polypeptide is a functional fragment or domain. In yet another preferred embodiment, the polypeptide is a chimera, where the chimera may include functional protein domains, including domains, repeats, post-translational modification sites, or other features. In a more preferred embodiment, the polypeptide is a plant polypeptide. In a more preferred embodiment, the plant is a dicot. In a more preferred embodiment, the plant is a gymnosperm. In a more preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum, and teosinte. In a most preferred embodiment, the cereal is rice.

In a preferred embodiment, the polypeptide is expressed in a specific location or tissue of a plant. In a more preferred embodiment, the location or tissue may be, for example, epidermis, root, vascular tissue, meristem, cambium, cortex, pith, leaf, and flower. In a more preferred embodiment, the location or tissue is a seed.

In a preferred embodiment, the polypeptide is involved in a function such as abiotic stress tolerance, disease resistance, enhanced yield or nutritional quality or composition.

In a preferred embodiment, the polypeptide sequence encoded by a nucleotide sequence having substantial similarity to a nucleotide sequence listed in odd numbered sequences of SEQ ID Nos:1-63 or a fragment, domain, or feature thereof or a sequence complementary thereto, includes a deletion or insertion of at least one nucleotide. In a more preferred embodiment, the deletion or insertion is of less than about thirty nucleotides. In a most preferred embodiment, the deletion or insertion is of less than about five nucleotides.

In a preferred embodiment, the polypeptide sequence encoded by a nucleotide sequence having substantial similarity to a nucleotide sequence listed in odd numbered sequences of SEQ ID Nos:1-63, or fragment, domain, or feature thereof or a sequence complementary-thereto, includes a substitution of at least one codon. In a more preferred embodiment, the substitution is conservative.

In a preferred embodiment, the polypeptide sequences having substantial similarity to the polypeptide sequence listed in even numbered sequences of SEQ ID Nos:2-64, or a fragment, domain, repeat, feature, or chimeras thereof includes a deletion or insertion of at least one amino acid.

In a preferred embodiment, the polypeptide sequences having substantial similarity to the polypeptide sequence listed in even numbered sequences of SEQ ID Nos:2-64, or a fragment, domain, repeat, feature, or chimeras thereof includes a substitution of at least one amino acid.

Embodiments of the present invention also relate to a shuffled nucleic acid containing a plurality of nucleotide sequence fragments, wherein at least one of the fragments corresponds to a region of a nucleotide sequence listed in odd numbered sequences of SEQ ID NOS:1-63, and wherein at least two of the plurality of sequence fragments are in an order, from 5′ to 3′ which is not an order in which the plurality of fragments naturally occur in a nucleic acid. In a more preferred embodiment, all of the fragments in a shuffled nucleic acid containing a plurality of nucleotide sequence fragments are from a single gene. In a more preferred embodiment, the plurality of fragments originates from at least two different genes. In a more preferred embodiment, the shuffled nucleic acid is operably linked to a promoter sequence. Another more preferred embodiment is a chimeric polynucleotide including a promoter sequence operably linked to the shuffled nucleic acid. In a more preferred embodiment, the shuffled nucleic acid is contained within a host cell.

Embodiments of the present invention also contemplate an expression cassette including a promoter sequence optably linked to an isolated nucleic acid containing a nucleotide sequence including:

(a) a nucleotide sequence listed in odd numbered sequences of SEQ ID NOS:1-63, or fragment, domain, or feature thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c).

Further encompassed within the invention is a recombinant vector comprising an expression cassette according to embodiments of the present invention. Also encompassed are plant cells, which contain expression cassettes, according to the present disclosure, and plants, containing these plant cells. In a preferred embodiment, the plant is a dicot. In another preferred embodiment, the plant is a gymnosperm. In another preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum and teosinte. In a most preferred embodiment, the cereal is rice.

In one embodiment, the expression cassette is expressed throughout the plant. In another embodiment, the expression cassette is expressed in a specific location or tissue of a plant. In a preferred embodiment, the location or tissue may be, for example, epidermis, root, vascular tissue, meristem, cambium, cortex, pith, leaf, and flower. In a more preferred embodiment, the location or tissue is a seed.

In one embodiment, the expression cassette is involved in a function such as, for example, but not limited to, disease resistance, yield, abiotic stress resistance, nutritional quality, carbon metabolism, photosynthesis, signal transduction, cell growth, reproduction, disease processes, gene regulation, and differentiation. In a more preferred embodiment, the chimeric polypeptide is involved in a function such as, abiotic stress tolerance, enhanced yield, disease resistance or nutritional composition.

In one embodiment, the plant contains a modification to a phenotype or measurable characteristic of the plant, the modification being attributable to theexpression cassette. In a preferred embodiment, the modification may be, for example, nutritional enhancement, increased nutrient uptake efficiency, enhanced production of endogenous compounds, and production of heterologous compounds. In another preferred embodiment, the modification includes having increased or decreased resistance to an herbicide, a stress, or a pathogen. In another preferred embodiment, the modification includes having enhanced or diminished requirement for light, water, nitrogen, or trace elements. In another preferred embodiment, the modification includes being enriched for an essential amino acid as a proportion of a protein fraction of the plant. In a more preferred embodiment, the protein fraction may be, for example, total seed protein, soluble protein, insoluble protein, water-extractable protein, and lipid-associated protein. In another preferred embodiment, the modification includes overexpression, underexpression, antisense modulation, sense suppression, inducible expression, inducible repression, or inducible modulation of a gene.

Embodiments of the present invention also provide seed and isolated product from plants which contain an expression cassette including a promoter sequence operably linked to an isolated nucleic acid containing a nucleotide sequence including:

(a) a nucleotide sequence listed in odd numbered sequences of SEQ ID NOS:1-63, or fragment, domain, or feature thereof;

(b) a nucleotide sequence encoding a polypeptide of even numbered sequences of SEQ ID NOS:2-64, fragment, domain or feature thereof;

(c) a nucleotide sequence having substantial similarity to (a) or (b);

(d) a nucleotide sequence capable of hybridizing to (a), (b) or (c);

(e) a nucleotide sequence complementary to (a), (b), (c) or (d); and

(f) a nucleotide sequence that is the reverse complement of (a), (b), (c) or (d) according to the present disclosure.

In a preferred embodiment the isolated product includes an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin and a plant hormone.

Embodiments of the present invention also relate to isolated products produced by expression of an isolated nucleic acid containing a nucleotide sequence including:

(a) a nucleotide sequence listed in odd numbered sequences of SEQ ID NOS:1-63, or fragment, domain, or feature thereof;

(b) a nucleotide sequence encoding a polypeptide listed in even numbered sequences of SEQ ID NOS: 2-64, or fragment, domain or feature thereof;

(c) a nucleotide sequence having substantial similarity to (a) or (b);

(d) a nucleotide sequence capable of hybridizing to (a) or (b);

(e) a nucleotide sequence complementary to (a), (b), (c) or (d); and

(f) a nucleotide sequence that is the reverse complement of (a), (b) (c) or (d) according to the present disclosure.

In a preferred embodiment, the product is produced in a plant. In another preferred embodiment, the product is produced in cell culture. In another preferred embodiment, the product is produced in a cell-free system. In another preferred embodiment, the product includes an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin and a plant hormone.

In a preferred embodiment, the product is a polypeptide containing an amino acid sequence listed in even numbered sequences of SEQ ID NOS:2-64. In a more preferred embodiment, the protein is an enzyme.

Embodiments of the present invention further relate to an isolated polynucleotide including a nucleotide sequence of at least 10 bases, which sequence is identical, complementary, or substantially similar to a region of any sequence of odd numbered sequences of SEQ ID NOS:1-63, and wherein the polynucleotide is adapted for any of numerous uses.

In a preferred embodiment, the polynucleotide is used as a chromosomal marker. In another preferred embodiment, the polynucleotide is used as a marker for RFLP analysis. In another preferred embodiment, the polynucleotide is used as a marker for quantitative trait linked breeding. In another preferred embodiment, the polynucleotide is used as a marker for marker-assisted breeding. In another preferred embodiment, the polynucleotide is used as a bait sequence in a two-hybrid system to identify sequence-encoding polypeptides interacting with the polypeptide encoded by the bait sequence. In another preferred embodiment, the polynucleotide is used as a diagnostic indicator for genotyping or identifying an individual or population of individuals. In another preferred embodiment, the polynucleotide is used for genetic analysis to identify boundaries of genes or exons.

Embodiments of the present invention also relate to an expression vector comprising or consisting of a nucleic acid molecule including:

(a) a nucleic acid encoding a polypeptide as listed in even numbered sequences of SEQ ID NOS:2-64;

(b) a fragment, one or more domains, or featured regions listed in odd numbered sequences of SEQ ID NOS:1-63; and

(c) a complete nucleic acid sequence listed in odd numbered sequences of SEQ ID NOS:1-63, or a fragment thereof, in combination with a heterologous sequence.

In a preferred embodiment, the expression vector includes one or more elements such as, for example, but not limited to, a promoter-enhancer sequence, a selection marker sequence, an origin of replication, an epitope-tag encoding sequence, or an affinity purification-tag encoding sequence. In a more preferred embodiment, the promoter-enhancer sequence may be, for example, the CaMV 35S promoter, the CaMV 19S promoter, the tobacco PR-1a promoter, ubiquitin and the phaseolin promoter. In another embodiment, the promoter is operable in plants, and more preferably, a constitutive or inducible promoter. In another preferred embodiment, the selection marker sequence encodes an antibiotic resistance gene. In another preferred embodiment, the epitope-tag sequence encodes V5, the peptide Phe-His-His-Thr-Thr, hemagglutinin, or glutathione-S-transferase. In another preferred embodiment the affinity purification-tag sequence encodes a polyamino acid sequence or a polypeptide. In a more preferred embodiment, the polyamino acid sequence is polyhistidine. In a more preferred embodiment, the polypeptide is chitin binding domain or glutathione-S-transferase. In a more preferred embodiment, the affinity purification-tag sequence comprises an intein encoding sequence.

In a preferred embodiment, the expression vector is a eukaryotic expression vector or a prokaryotic expression vector. In a more preferred embodiment, the eukaryotic expression vector includes a tissue-specific promoter. More preferably, the expression vector is operable in plants.

Embodiments of the present invention also relate to a cell comprising or consisting of a nucleic acid construct comprising an expression vector and a nucleic acid including a nucleic acid encoding a polypeptide as listed in even numbered sequences of SEQ ID NOS:2-64, or a nucleic acid sequence listed in odd numbered sequences of SEQ ID NOS:1-63, or a segment thereof, in combination with a heterologous sequence.

In a preferred embodiment, the cell is a bacterial cell, a fungal cell, a plant cell, or an animal cell. In a more preferred embodiment, the polypeptide is expressed in a specific location or tissue of a plant. In a most preferred embodiment, the location or tissue may be, for example, epidermis, root, vascular tissue, meristem, cambium, cortex, pith, leaf, and flower. In an alternate most preferred embodiment, the location or tissue is a seed. In a preferred embodiment, the polypeptide is involved in a function such as, for example, carbon metabolism, photosynthesis, signal transduction, cell growth, reproduction, disease processes, gene regulation, and differentiation. More preferably, the polypeptide is involved in a function such as, abiotic stress tolerance, enhanced yield, disease resistance or nutritional composition.

Embodiments of the present invention also relate to polypeptides encoded by the isolated nucleic acid molecules of the present disclosure including a polypeptide containing a polypeptide sequence encoded by an isolated nucleic acid containing a nucleotide sequence including:

(a) a nucleotide sequence listed in odd numbered sequences of SEQ ID NOS:1-63, or exon, domain, or feature thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c);

(f) or a functional fragment thereof.

A polypeptide containing a polypeptide sequence encoded by an isolated nucleic acid containing a nucleotide sequence, its complement, or its reverse complement, encoding a polypeptide including a polypeptide sequence including:

(a) a polypeptide sequence listed in even numbered sequences of SEQ ID NOS:2-64, or a domain, repeat, feature, or chimeras thereof;

(b) a polypeptide sequence having substantial similarity to (a);

(c) a polypeptide sequence encoded by a nucleotide sequence identical to or having substantial similarity to a nucleotide sequence listed in odd numbered sequences of SEQ ID NOS:1-63, or an exon, domain, or feature thereof, or a sequence complementary thereto;

(d) a polypeptide sequence encoded by a nucleotide sequence capable of hybridizing under medium stringency conditions to a nucleotide sequence listed in odd numbered sequences of SEQ ID NOS:1-63, or to a sequence complementary thereto; and

(e) a functional fragment of (a), (b), (c) or (d);

(f) or a functional fragment thereof.

Embodiments of the present invention contemplate a polypeptide containing a polypeptide sequence encoded by an isolated nucleic acid which includes a shuffled nucleic acid containing a plurality of nucleotide sequence fragments, wherein at least one of the fragments corresponds to a region of a nucleotide sequence listed in odd numbered sequences of SEQ ID NOS:1-63, and wherein at least two of the plurality of sequence fragments are in an order, from 5′ to 3′ which is not an order in which the plurality of fragments naturally occur in a nucleic acid, or functional fragment thereof.

Embodiments of the present invention contemplate a polypeptide containing a polypeptide sequence encoded by an isolated polynucleotide containing a nucleotide sequence of at least 10 bases, which sequence is identical, complementary, or substantially similar to a region of any of sequences of odd numbered sequences of SEQ ID NOS:1-63, and wherein the polynucleotide is adapted for a use including:

(a) use as a chromosomal marker to identify the location of the corresponding or complementary polynucleotide on a native or artificial chromosome;

(b) use as a marker for RFLP analysis;

(c) use as a marker for quantitative trait linked breeding;

(d) use as a marker for marker-assisted breeding;

(e) use as a bait sequence in a two-hybrid system to identify sequence encoding polypeptides interacting with the polypeptide encoded by the bait sequence;

(f) use as a diagnostic indicator for genotyping or identifying an individual or population of individuals; and

(g) use for genetic analysis to identify boundaries of genes or exons;

(h) or functional fragment thereof.

Embodiments of the present invention also contemplate an isolated polypeptide containing a polypeptide sequence including:

(a) a polypeptide sequence listed in even numbered sequences of SEQ ID NOS:2-64, or exon, domain, or feature thereof;

(b) a polypeptide sequence having substantial similarity to (a);

(c) a polypeptide sequence encoded by a nucleotide sequence identical to or having substantial similarity to a nucleotide sequence listed in odd numbered sequences of SEQ ID NOS:1-63, or an exon, domain, or feature thereof, or a sequence complementary thereto;

(d) a polypeptide sequence encoded by a nucleotide sequence capable of hybridizing under medium stringency conditions to a nucleotide sequence listed in odd numbered sequences of SEQ ID NOS:1-63, or to a sequence complementary thereto; and

(e) a functional fragment of (a), (b), (c) or (d).

In a preferred embodiment, the substantial similarity is at least about 65% identity. In a more preferred embodiment, the substantial similarity is at least about 80% identity. In a most preferred embodiment, the substantial similarity is at least about 95% identity. In a preferred embodiment, the substantial similarity is at least three percent greater than the percent identity to the closest homologous sequence listed in any of the Tables.

In a preferred embodiment, the sequence having substantial similarity is from a plant. In a more preferred embodiment, the plant is a dicot. In a more preferred embodiment, the plant is a gymnosperm. In a more preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum and teosinte. In a most preferred embodiment, the cereal is rice.

In a preferred embodiment, the polypeptide is expressed in a specific location or tissue of a plant. In a more preferred embodiment, the location or tissue may be, for example, epidermis, root, vascular tissue, meristem, cambium, cortex, pith, leaf, and flower. In a more preferred embodiment, the location or tissue is a seed. In a preferred embodiment, the polypeptide is involved in a function such as, for example, carbon metabolism, photosynthesis, signal transduction, cell growth, reproduction, disease processes, gene regulation, and differentiation.

In a preferred embodiment, hybridization of a polypeptide sequence encoded by a nucleotide sequence identical to or having substantial similarity to a nucleotide sequence listed in odd numbered sequences of SEQ ID NOS:1-63, or an exon, domain, or feature thereof, or a sequence complementary thereto, or a polypeptide sequence encoded by a nucleotide sequence capable of hybridizing under medium stringency conditions to a nucleotide sequence listed in odd numbered sequences of SEQ ID NOS:1-63, or to a sequence complementary thereto, allows the sequence to form a duplex atmedium or high stringency.

In a preferred embodiment, a polypeptide having substantial similarity to a polypeptide sequence listed in even numbered sequences of SEQ ID NOS:2-64, or exon, domain, or feature thereof, is an allelic variant of the polypeptide sequence listed in even numbered sequences of SEQ ID NOS:2-64. In another preferred embodiment, a polypeptide having substantial similarity to a polypeptide sequence listed in even numbered sequences of SEQ ID NOS:2-64, or exon, domain, or feature thereof, is a naturally occurring variant of the polypeptide sequence listed in even numbered sequences of SEQ ID NOS:2-64. In another preferred embodiment, a polypeptide having substantial similarity to a polypeptide sequence listed in even numbered sequences of SEQ ID NOS:2-64, or exon, domain, or feature thereof, is a polymorphic variant of the polypeptide sequence listed in even numbered sequences of SEQ ID NOS:2-64.

In an alternate preferred embodiment, the sequence having substantial similarity contains a deletion or insertion of at least one amino acid. In a more preferred embodiment, the deletion or insertion is of less than about ten amino acids. In a most preferred embodiment, the deletion or insertion is of less than about three amino acids.

In a preferred embodiment, the sequence having substantial similarity encodes a substitution in at least one amino acid.

Also contemplated is a method of producing a plant comprising a modification thereto, including the steps of: (1) providing a nucleic acid which is an isolated nucleic acid containing a nucleotide sequence including:

(a) a nucleotide sequence listed in odd numbered sequences of SEQ ID NOS:1-63, or exon, domain, or feature thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c); and (2) introducing the nucleic acid into the plant, wherein the nucleic acid is expressible in the plant in an amount effective to effect the modification. In one embodiment, the modification comprises an altered trait in the plant, wherein the trait corresponds to the nucleic acid introduced into the plant. In other preferred embodiments, the altered trait is related to a feature listed in any of Tables 1-4, and it is particularly preferred when the trait corresponds to disease resistance, yield, abiotic stress resistance, nutritional composition, carbon metabolism, photosynthesis, signal transduction, cell growth, reproduction, disease processes, or differentiation.

In another embodiment, the modification includes an increased or decreased expression or accumulation of a product of the plant. Preferably, the product is a natural product of the plant. Equally preferably, the product is a new or altered product of the plant. Preferably, the product includes, but is not limited to, an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin and a plant hormone. Another preferred embodiment provides method of controlling a pathogen by delivering an effective amount of a product resulting from modification of the plant.

Embodiments of the present invention also include a method of controlling a pathogen sensitive to a product, including expressing an isolated nucleic acid containing a nucleotide sequence including:

(a) a nucleotide sequence listed odd numbered sequences of SEQ ID Nos:1-63, or exon, domain, or feature thereof;

(b) a nucleotide sequence having substantial similarity to (a);

(c) a nucleotide sequence capable of hybridizing to (a);

(d) a nucleotide sequence complementary to (a), (b) or (c); and

(e) a nucleotide sequence which is the reverse complement of (a), (b) or (c); thereby causing the plant to produce the product. In preferred embodiments, the product is selected from the group consisting of an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin and a plant hormone.

Also encompassed within the presently disclosed invention is a method of producing a recombinant protein, comprising the steps of:

(a) growing recombinant cells comprising a nucleic acid construct under suitable growth conditions, the construct comprising an expression vector and a nucleic acid including: a nucleic acid encoding a protein as listed in even numbered nucleotide sequences of SEQ ID NOS:2-64, or a nucleic acid sequence listed in odd numbered nucleotide sequences of SEQ ID NOS:1-63, or segments thereof; and

(b) isolating from the recombinant cells the recombinant protein expressed thereby.

Embodiments of the present invention provide a method of producing a recombinant protein in which the expression vector includes one or more elements including a promoter-enhancer sequence, a selection marker sequence, an origin of replication, an epitope-tag encoding sequence, and an affinity purification-tag encoding sequence. In one preferred embodiment, the nucleic acid construct includes an epitope-tag encoding sequence and the isolating step includes use of an antibody specific for the epitope-tag. In another preferred embodiment, the nucleic acid construct contains a polyamino acid encoding sequence and the isolating step includes use of a resin comprising a polyamino acid binding substance, preferably where the polyamino acid is polyhistidine and the polyamino binding resin is nickel-charged agarose resin. In yet another preferred embodiment, the nucleic acid construct contains a polypeptide encoding sequence and the isolating step includes the use of a resin containing a polypeptide binding substance, preferably where the polypeptide is a chitin binding domain and the resin contains chitin-sepharose.

Embodiments of the present invention also relate to a plant modified by a method that includes introducing into a plant a nucleic acid where the nucleic acid is expressible in the plant in an amount effective to effect the modification. The modification can be, for example, nutritional enhancement, increased nutrient uptake efficiency, enhanced production of endogenous compounds, and production of heterologous compounds. In one embodiment, the modified plant has increased or decreased resistance to an herbicide, a stress, or a pathogen. In another embodiment, the modified plant has enhanced or diminished requirement for light, water, nitrogen, or trace elements. In yet another embodiment, the modified plant is enriched for an essential amino acid as a proportion of a protein fraction of the plant. The protein fraction may be, for example, total seed protein, soluble protein, insoluble protein, water-extractable protein, and lipid-associated protein. The modification may include overexpression, underexpression, antisense modulation, sense suppression, inducible expression, inducible repression, or inducible modulation of a gene.

The invention further relates to a seed from a modified plant or an isolated product of a modified plant, where the product may be an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin and a plant hormone.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Further aspects, features and advantages of this invention will become apparent from the detailed description of the preferred embodiments that follow.

### BRIEF DESCRIPTION OF THE SEQUENCES IN THE SEQUENCE LIST

Odd numbered SEQ ID NOs:1-63 are nucleotide sequences isolated from Oryza sativa that are more fully described in Tables 1-4 below.

Even numbered SEQ ID Nos: 2-64 are protein sequences encoded by the immediately preceding nucleotide sequence, e.g., SEQ ID NO:2 is the protein encoded by the nucleotide sequence of SEQ ID NO:1, SEQ ID NO:4 is the protein encoded by the nucleotide sequence of SEQ ID NO:3, etc.

### Definitions

For clarity, certain terms used in the specification are defined and presented as follows:

“Associated with/operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

A “chimeric construct” is a recombinant nucleic acid sequence in which a promoter or regulatory nucleic acid sequence is operatively linked to, or associated with, a nucleic acid sequence that codes for an mRNA or which is expressed as a protein, such that the regulatory nucleic acid sequence is able to regulate transcription or expression of the associated nucleic acid sequence. The regulatory nucleic acid sequence of the chimeric construct is not normally operatively linked to the associated nucleic acid sequence as found in nature.

Co-factor: natural reactant, such as an organic molecule or a metal ion, required in an enzyme-catalyzed reaction. A co-factor is e.g. NAD(P), riboflavin (including FAD and FMN), folate, molybdopterin, thiamin, biotin, lipoic acid, pantothenic acid and coenzyme A, S-adenosylmethionine, pyridoxal phosphate, ubiquinone, menaquinone. Optionally, a co-factor can be regenerated and reused.

A “coding sequence” is a nucleic acid sequence that is transcribed into RNA such as mRNA, rRNA, tRNA, snRNA, sense RNA or antisense RNA. Preferably the RNA is then translated in an organism to produce a protein.

Complementary: “complementary” refers to two nucleotide sequences that comprise antiparallel nucleotide sequences capable of pairing with one another upon formation of hydrogen bonds between the complementary base residues in the antiparallel nucleotide sequences.

Enzyme activity: means herein the ability of an enzyme to catalyze the conversion of a substrate into a product. A substrate for the enzyme comprises the natural substrate of the enzyme but also comprises analogues of the natural substrate, which can also be converted, by the enzyme into a product or into an analogue of a product. The activity of the enzyme is measured for example by determining the amount of product in the reaction after a certain period of time, or by determining the amount of substrate remaining in the reaction mixture after a certain period of time. The activity of the enzyme is also measured by determining the amount of an unused co-factor of the reaction remaining in the reaction mixture after a certain period of time or by determining the amount of used co-factor in the reaction mixture after a certain period of time. The activity of the enzyme is also measured by determining the amount of a donor of free energy or energy-rich molecule (e.g. ATP, phosphoenolpyruvate, acetyl phosphate or phosphocreatine) remaining in the reaction mixture after a certain period of time or by determining the amount of a used donor of free energy or energy-rich molecule (e.g. ADP, pyruvate, acetate or creatine) in the reaction mixture after a certain period of time.

Expression Cassette: “Expression cassette” as used herein means a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular external stimulus. In the case of a multicellular organism, such as a plant, the promoter can also be specific to a particular tissue or organ or stage of development.

Gene: the term “gene” is used broadly to refer to any segment of DNA associated with a biological function. Thus, genes include coding sequences and/or the regulatory sequences required for their expression. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

Heterologous/exogenous: The terms “heterologous” and “exogenous” when used herein to refer to a nucleic acid sequence (e.g. a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” nucleic acid (e.g. DNA) sequence is a nucleic acid (e.g. DNA) sequence naturally associated with a host cell into which it is introduced.

Hybridization: The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

Inhibitor: a chemical substance that inactivates the enzymatic activity of a protein such as a biosynthetic enzyme, receptor, signal transduction protein, structural gene product, or transport protein. The term “herbicide”(or “herbicidal compound” is used herein to define an inhibitor applied to a plant at any stage of development, whereby the herbicide inhibits the growth of the plant or kills the plant.

Interaction: quality or state of mutual action such that the effectiveness or toxicity of one protein or compound on another protein is inhibitory (antagonists) or enhancing (agonists).

A nucleic acid sequence is “isocoding with” a reference nucleic acid sequence when the nucleic acid sequence encodes a polypeptide having the same amino acid sequence as the polypeptide encoded by the reference nucleic acid sequence.

Isogenic: plants that are genetically identical, except that they may differ by the presence or absence of a heterologous DNA sequence.

Isolated: in the context of the present invention, an isolated DNA molecule or an isolated enzyme is a DNA molecule or enzyme that, by the hand of man, exists apart from its native environment and is therefore not a product of nature. An isolated DNA molecule or enzyme may exist in a purified form or may exist in a non-native environment such as, for example, in a transgenic host cell.

Mature protein: protein from which the transit peptide, signal peptide, and/or propeptide portions have been removed.

Minimal Promoter the smallest piece of a promoter, such as a TATA element, that can support any transcription. A minimal promoter typically has greatly reduced promoter activity in the absence of upstream activation. In the presence of a suitable transcription factor, the minimal promoter functions to permit transcription.

Modified Enzyme Activity: enzyme activity different from that which naturally occurs in a plant (i.e. enzyme activity that occurs naturally in the absence of direct or indirect manipulation of such activity by man), which is tolerant to inhibitors that inhibit the naturally occurring enzyme activity.

Native: refers to a gene that is present in the genome of an untransformed plant cell.

Naturally occurring: the term “naturally occurring” is used to describe an object that can be found in nature as distinct from being artificially produced by man. For example, a protein or nucleotide sequence present in an organism (including a virus), which can be isolated from a source in nature and which has not been intentionally modified by man in the laboratory, is naturally occurring.

Nucleic acid: the term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al, Nucleic Acid Res. 19: 5081 (1991); Ohtsuka et al., J. Biol. Chem. 260: 2605-2608 (1985); Rossolini et al., Mol. Cell. Probes 8: 91-98 (1994)). The terms “nucleic acid” or “nucleic acid sequence” may also be used interchangeably with gene, cDNA, and mRNA encoded by a gene.

“ORF” means open reading frame.

Percent identity: the phrases “percent identical” or “percent identical,” in the context of two nucleic acid or protein sequences, refers to two or more sequences or subsequences that have for example 60%, preferably 70%, more preferably 80%, still more preferably 90%, even more preferably 95%, and most preferably at least 99% nucleotide or amino acid residue identity, when compared and aligned for maximum correspondence, as measured using one of the following sequence comparison algorithms or by visual inspection. Preferably, the percent identity exists over a region of the sequences that is at least about 50 residues in length, more preferably over a region of at least about 100 residues, and most preferably the percent identity exists over at least about 150 residues. In an especially preferred embodiment, the percent identity exists over the entire length of the coding regions.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Pre-protein: protein that is normally targeted to a cellular organelle, such as a chloroplast, and still comprises its native transit peptide.

Purified: the term “purified,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least about 50% pure, more preferably at least about 85% pure, and most preferably at least about 99% pure.

Two nucleic acids are “recombined” when sequences from each of the two nucleic acids are combined in a progeny nucleic acid. Two sequences are “directly” recombined when both of the nucleic acids are substrates for recombination. Two sequences are “indirectly recombined” when the sequences are recombined using an intermediate such as a cross-over oligonucleotide. For indirect recombination, no more than one of the sequences is an actual substrate for recombination, and in some cases, neither sequence is a substrate for recombination.

“Regulatory elements” refer to sequences involved in controlling the expression of a nucleotide sequence. Regulatory elements comprise a promoter operatively linked to the nucleotide sequence of interest and termination signals. They also typically encompass sequences required for proper translation of the nucleotide sequence.

Significant Increase: an increase in enzymatic activity that is larger than the margin of error inherent in the measurement technique, preferably an increase by about 2-fold or greater of the activity of the wild-type enzyme in the presence of the inhibitor, more preferably an increase by about 5-fold or greater, and most preferably an increase by about 10-fold or greater.

Significantly less: means that the amount of a product of an enzymatic reaction is reduced by more than the margin of error inherent in the measurement technique, preferably a decrease by about 2-fold or greater of the activity of the wild-type enzyme in the absence of the inhibitor, more preferably an decrease by about 5-fold or greater, and most preferably an decrease by about 10-fold or greater.

Specific Binding/Immunological Cross-Reactivity: An indication that two nucleic acid sequences or proteins are substantially identical is that the protein encoded by the first nucleic acid is immunologically cross reactive with, or specifically binds to, the protein encoded by the second nucleic acid. Thus, a protein is typically substantially identical to a second protein, for example, where the two proteins differ only by conservative substitutions. The phrase “specifically (or selectively) binds to an antibody,” or “specifically (or selectively) immunoreactive with,” when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised to the protein with the amino acid sequence encoded by any of the nucleic acid sequences of the invention can be selected to obtain antibodies specifically immunoreactive with that protein and not with other proteins except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See Harlow and Lane (1988) Antibodies, A Laboratory Manual, Cold Spring Harbor Publications, New York “Harlow and Lane”), for a description of immunoassay formats and conditions that can be used to determine specific immunoreactivity. Typically a specific or selective reaction will be at least twice background signal or noise and more typically more than 10 to 100 times background.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% fonmamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences of the present invention: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

A “subsequence” refers to a sequence of nucleic acids or amino acids that comprise a part of a longer sequence of nucleic acids or amino acids (e.g., protein) respectively.

Substrate: a substrate is the molecule that an enzyme naturally recognizes and converts to a product in the biochemical pathway in which the enzyme naturally carries out its function, or is a modified version of the molecule, which is also recognized by the enzyme and is converted by the enzyme to a product in an enzymatic reaction similar to the naturally-occurring reaction.

Transformation: a process for introducing heterologous DNA into a plant cell, plant tissue, or plant. Transformed plant cells, plant tissue, or plants are understood to encompass not only the end product of a transformation process, but also transgenic progeny thereof.

“Transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells, tissues, or plants are understood to encompass not only the end product of a transformation process, but also transgenic progeny thereof. A “non-transformed,” “non-transgenic,” or “non-recombinant” host refers to a wild-type organism, e.g., a bacterium or plant, which does not contain the heterologous nucleic acid molecule.

Viability: “viability” as used herein refers to a fitness parameter of a plant. Plants are assayed for their homozygous performance of plant development, indicating which proteins are essential for plant growth.

### DETAILED DESCRIPTION OF THE INVENTION

### I. General Description of Trait Functional Genomics Project

The goal of functional genomics is to assign functions to the genes of an organism using a variety of methodologies, including but not limited to bioinformatics, gene expression studies, gene and gene product interactions, genetics, biochemistry and molecular genetics. For example, bioinformatics can assign function to a given gene by identifying genes in heterologous organisms with a high degree of similarity (homology) at the amino acid or nucleotide level. Expression of a gene at the mRNA or protein levels can assign function by linking expression of a gene to an environmental response, a developmental process or a genetic (mutational) or molecular genetic (gene overexpression or underexpression) perturbation. Expression of a gene at the mRNA level can be ascertained either alone (Northern analysis) or in concert with other genes (microarray analysis), whereas expression of a gene at the protein level can be ascertained either alone (native or denatured protein gel or immunoblot analysis) or in concert with other genes (proteomic analysis). Knowledge of protein/protein and protein/DNA interactions can assign function by identifying proteins and nucleic acid sequences acting together in the same biological process. Genetics can assign function to a gene by demonstrating that DNA lesions (mutations) in the gene have a quantifiable effect on the organism, including but not limited to: its development; hormone biosynthesis and response; growth and growth habit (plant architecture); mRNA expression profiles; protein expression profiles; ability to resist diseases; tolerance of abiotic stresses; ability to acquire nutrients; photosynthetic efficiency; altered primary and secondary metabolism; and the composition of various plant organs. Biochemistry can assign function by demonstrating that the protein encoded by the gene, typically when expressed in a heterologous organism, possesses a certain enzymatic activity, alone or in combination with other proteins. Molecular genetics can assign function by overexpressing or underexpressing the gene in the native plant or in heterologous organisms, and observing quantifiable effects as described in functional assignment by genetics above.

It is recognized by those skilled in the art that these different methodologies can each provide data as evidence for the function of a particular gene, and that such evidence is stronger with increasing amounts of data used for functional assignment: preferably from a single methodology, more preferably from two methodologies, and even more preferably from more than two methodologies. In addition, those skilled in the art are aware that different methodologies can differ in the strength of the evidence for the assignment of gene function. Typically, but not always, a datum of biochemical, genetic and molecular genetic evidence is considered stronger than a datum of bioinformatic or gene expression evidence. Finally, those skilled in the art recognize that, for different genes, a single datum from a single methodology can differ in terms of the strength of the evidence provided by each distinct datum for the assignment of the function of these different genes.

The objective of trait functional genomics is to identify crop trait genes, i.e. genes capable of conferring useful agronomic traits in crop plants. Such agronomic traits include, but are not limited to: enhanced yield, whether in quantity or quality; enhanced nutrient acquisition and enhanced metabolic efficiency; enhanced or altered nutrient composition of plant tissues used for food, feed, fiber or processing; enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. The deployment of such identified trait genes could materially improve crop plants for the benefit of agriculture, potentially, irrespective of the method of deployment of such genes.

Cereals are the most important crop plants on the planet, in terms of both human and animal consumption. Genomic synteny (conservation of gene order within large chromosomal segments) is observed in the rice, maize, wheat, barley, rye, oats and other agriculturally important monocots, which facilitates the mapping and isolation of orthologous genes from diverse cereal species based on the sequence of a single cereal gene. Rice has the smallest (˜420 Mb) genome among the cereal grains, and has recently been a major focus of public and private genomic and EST sequencing efforts.

To identify crop trait genes in the rice genome, genes with likely or demonstrated effects on agronomic traits of interest as defined above were identified in the scientific literature. The predicted peptides encoded by these genes were then used to search a proprietary database of rice genomic sequences for those with high similarity, using search algorithms familiar to those skilled in the art, resulting in the identification of rice trait gene orthologs. Rice trait gene orthologs were assigned function based on similarity searches of two different public databases: the SwissProt protein database and the GenPept non-redundant (nr) database of conceptual translations of all of the nucleotide sequences in Genbank.

To demonstrate the validity of this approach, and to provide additional evidence for the function of a subset of these genes, full-length and partial cDNAs of rice trait gene orthologs were isolated. Several different commercially available gene prediction programs were used to help predict full-length cDNAs corresponding to the putative rice trait gene orthologs. Full-length and partial cDNAs were isolated based on these predictions, using two different approaches. In one approach, a similarity search algorithm was used to search a database of sequenced cDNA clones. In another approach, the predicted cDNAs were used in combination with the genomic sequence to design primers for PCR amplification using a commercially available PCR primer-picking program. Primers were use d for PCR amplification of full-length or partial cDNAs from rice cDNA libraries or first-strand cDNA. cDNA clones resulting from either approach were used for the construction of vectors designed for overexpression or underexpression of corresponding genes in transgenic rice plants. Assays to identify transgenic plants for alterations in traits of interest are to be used to unambiguously assign the utility of these genes for the improvement of rice, and by extension, other cereals, either by transgenic or classical breeding methods.

### II. Identifying, Cloning and Sequencing cDNAs

The identification of genes of interest and determination of cDNA homologies is set forth in Example 1. The cloning and sequencing of the cDNAs of the present invention are described in Example 2.

The isolated nucleic acids and proteins of the present invention are usable over a range of plants, monocots and dicots, in particular monocots such as rice, wheat, barley and maize. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum sp., or teosinte. In a most preferred embodiment, the cereal is rice. Other plants genera include, but are not limited to, Cucurbita, Rosa, Vitis, Juglans, Gragaria, Lotus, Medicago, Onobrychis; Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium, and Triticum.

The present invention also provides a method of genotyping a plant or plant part comprising a nucleic acid molecule of the present invention. Optionally, the plant is a monocot such as, but not limited rice or wheat. Genotyping provides a means of distinguishing homologs of a chromosome pari and can be used to differentiate segregants in a plant population. Molecular marker methods can be used in phylogenetic studies, characterizing genetic relationships among crop varieties, identifying crosses or somatic hybrids, localizing chromosomeal segments affecting mongenic traits, map based cloning, and the study of quantitative inheritance (see Plant Molecular Biology: A Laboratory Manual, Chapter 7, Clark ed., Springer-Verlag, Berlin 1997; Paterson, A. H., “The DNA Revolution”, chapter 2 in Genome Mapping in Plants, Paterson, A. H. ed., Academic Press/R. G. Lands Co., Austin, Tex. 1996).

The method of genotyping may employ any number of molecular marker analytical techniques such as, but not limited to, restriction length polymorphisms (RFLPs). As is well known in the art, RFLPs are produced by differences in the DNA restriction fragment lengths resulting from nucleotide differences between alleles of the same gene. Thus, the present invention provides a method of following segregation of a gene or nucleic acid of the present invention or chromosomal sequences genetically linked by using RFLP analysis. Linked chromosomal sequences are within 50 centiMorgans (50 cM), within 40 or 30 cM, preferably within 20 or 10 cM, more preferably within 5, 3, 2, or 1 cM of the nucleic acid of the invention.

### III. Traits of Interest

The present invention encompasses the identification and isolation of cDNAs encoding genes of interest in the trait areas of abiotic stress tolerance, enhanced yield, disease resistance, and nutritional composition. Abiotic stresses such as, but not limited to, cold, heat, drought or salt stress can significantly affect the growth and/or yield of plants. Additionally, altering the expression of genes related to these traits are used to improve or modify the rice plants and/or grain as desired. Examples 3-7 describe the isolated genes of interest and methods of analyzing the alteration of expression and their effects on the plant characteristics.

One aspect of the present invention provides compositions and methods for altering (i.e. increasing or decreasing) the level of nucleic acid molecules and polypeptides of the present invention in plants. In particular, the nucleic acid molecules and polypeptides of the invention are expressed constitutively, temporally or spatially, e.g. at developmental stages, in certain tissues, and/or quantities, which are uncharacteristic of non-recombinantly engineered plants. Therefore, the present invention provides utility in such exemplary applications as altering the specified characteristics identified above.

Pathogens of the invention include, but are not limited to, fungi, bacteria, nematodes, viruses or viroids, etc.

Generally Viruses include tobacco or cucumber mosaic virus, ringspot virus, necrosis virus, maize dwarf mosaic virus, etc. Specific fungal, bacterial and viral pathogens of major crops include, but are not limited to: RICE: rice brown spot fungus (Cochliobolus miyabeanus), rice blast fungus—Magnaporthe grisea (Pyricularia grisea), Magnaporthe salvinii (Sclerotium oryzae), Xanthomomas oryzae pv. oryzae, Xanthomomas oryzae pv. oryzicola, Rhizoctonia spp. (including but not limited to Rhizoctonia solani, Rhizoctonia oryzae and Rhizoctonia oryzae-sativae), Pseudomonas spp. (including but not limited to Pseudomonas plantarii, Pseudomonas avenae, Pseudomonas glumae, Pseudomonas fuscovaginae, Pseudomonas alboprecipitans, Pseudomonas syringae pv. panici, Pseudomonas syringae pv. syringae, Pseudomonas syringae pv. oryzae and Pseudomonas syringae pv. aptata), Erwinia spp. (including but not limited to Erwinia herbicola, Erwinia amylovaora, Erwinia chrysanthemi and Erwinia carotovora), Achyla spp. (including but not limited to Achyla conspicua and Achyia klebsiana), Pythium spp. (including but not limited to Pythium dissotocum, Pythium irregulare, Pythium arrhenomanes, Pythium myriotylum, Pythium catenulatum, Pythium graminicola and Pythium spinosum), Saprolegnia spp., Dictyuchus spp., Pythiogeton spp., Phytophthora spp., Alternaria padwickii, Cochliobolus miyabeanus, Curvularia spp. (including but not limited to Curvularia lunata, Curvularia affinis, Curvularia clavata, Curvularia eragrostidis, Curvularia fallax, Curvularia geniculata, Curvularia inaequalis, Curvularia intermedia, Curvularia oryzae, Curvularia oryzae-sativae, Curvularia pallescens, Curvularia senegalensis, Curvularia tuberculata, Curvularia uncinata and Curvularia verruculosa), Sarocladium oryzae, Gerlachia oryzae, Fusarium spp. (including but not limited Fusarium graminearum, Fusarium nivale and to different pathovars of Fusarium monoliforme, including pvs. fujikuroi and zeae), Sclerotium rolfsii, Phoma exigua, Mucor fragilis, Trichoderma viride, Rhizopus spp., Cercospora oryzae, Entyloma oryzae, Dreschlera gigantean, Scierophthora macrospora, Mycovellosiella oryzae, Phomopsis oryzae-sativae, Puccinia graminis, Uromyces coronatus, Cylindrocladium scoparium, Sarocladium oryzae, Gaeumannomyces graminis pv. graminis, Myrothecium verrucaria, Pyrenochaeta oryzae, Ustilaginoidea virens, Neovossia spp. (including but not limited to Neovossia horrida), Tilletia spp., Balansia oryzae-sativae, Phoma spp. (including but not limited to Phoma sorghina, Phoma insidiosa, Phoma glumarum, Phoma glumicola and Phoma oryzina), Nigrospora spp. (including but not limited to Nigrospora oryzae, Nigrospora sphaerica, Nigrospora panici and Nigrospora padwickii), Epiococcum nigrum, Phyllostica spp., Wolkia decolorans, Monascus purpureus, Aspergillus spp., Penicillium spp., Absidia spp., Mucor spp., Chaetomium spp., Dematium spp., Monilia spp., Streptomyces spp., Syncephalastrum spp., Verticillium spp., Nematospora coryli, Nakataea sigmoidea, Cladosporium spp., Bipolaris spp., Coniothyrium spp., Diplodia oryzae, Exserophilum rostratum, Helococera oryzae, Melanomma glumarum, Metashaeria spp., Mycosphaerella spp., Oidium spp., Pestalotia spp., Phaeoseptoria spp., Sphaeropsis spp., Trematosphaerella spp., rice black-streaked dwarf virus, rice dwarf virus, rice gall dwarf virus, barley yellow dwarf virus, rice grassy stunt virus, rice hoja blanca virus, rice necrosis mosaic virus, rice ragged stunt virus, rice stripe virus, rice stripe necrosis virus, rice transitory yellowing virus, rice tungro bacilliform virus, rice tungro spherical virus, rice yellow mottle virus, rice tarsonemid mite virus, Echinochloa hoja blanca virus, Echinochloa ragged stunt virus, orange leaf mycoplasma-like organism, yellow dwarf mycoplasma-like organism, Aphelenchoides besseyi, Ditylenchus angustus, Hirschmanniella spp., Criconemella spp., Meloidogyne spp., Heterodera spp., Pratylenchus spp., Hoplolaimus indicus:

SOYBEANS: Phytophthora sojae, Fusarium solani f. sp. Glycines, Macrophomina phaseolina, Fusarium, Pythium, Rhizoctonia, Phialophora gregata, Sclerotinia sclerotiorum, Diaporthe phaseolorum var. sojae, Colletotrichum truncatum, Phomopsis longicolla, Cercospora kikuchii, Diaporthe phaseolonum var. meridionalis (and var. caulivora), Phakopsora pachyrhyzi, Fusarium solani, Microsphaera diffusa, Septoria glycines, Cercospora kikuchii, Macrophomina phaseolina, Sclerotinia sclerotiorum, Corynespora cassiicola, Rhizoctonia solani, Cercospora sojina,Phytophthora megasperma fsp. glycinea, Macrophomina phaseolina, Fusarium oxysporum, Diapothe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassiicola, Phyllosticta sojicola, Alternaria alternata, Pseudomonas syringae p.v. glycinea, Xanthomonas campestris p.v. phaseoli, Microspaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium dearyanum, Tomato spotted wilted virus, Heterodera glycines, Fusarium solani, Soybean cyst and root knot nematodes.

CORN: Fusarium moniliforme var. subglutinans, Erwinia stewartii, Fusarium moniliforme, Gibberella zeae (Fusarium Graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (cochliobolus heterostrophus), Helminthosporium carbonum I, II, and III (Cochliobolus carbonum), Exserohilum turcicum I, II and III, Helminthosporium pedicellatum, Physoderma maydis, Phyllosticta maydis, Kabatie-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganese subsp. Nebraskense, Trichoderma viride, Maize dwarf Mosaic Virus A and B, Wheat Streak Mosaic Virus, Maize Chlorotic Dwarf Virus, Claviceps sorghi, Pseudonomas avenae, Erwinia chrysantemi p.v. Zea, Erwinia corotovora, Cornstun spiroplasma, Diplodia macrospora, Sclerophthora macrospora, Peronosclerospora sorghi, Peronoscherospora philippinesis, Peronosclerospora maydis, Peronosclerospora sacchari, Spacelotheca reiliana, Physopella zea, Cephalosporium maydis, Caphalosporium acremonium, Maize Chlorotic Mottle Virus, High Plains Virus, Maize Mosaic Virus, Maize Rayado Fino Virus, Maize Streak Virus, Maize Stripe Virus, Maize Rought Dwarf Virus:

WHEAT: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f. sp. Tritici, Puccinia graminis f. sp. Tritici, Puccinia recondite f. sp. tritici, puccinia striiformis, Pyrenophora triticirepentis, Septoria nodorum, Septoria tritici, Spetoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Virus, Wheat Spindle Streak Virus, American Wheat Striate Virus, Claviceps purpurea, Tilletia tritici, Tilletia laevis, Pstilago tritici, Tilletia indica, Rhizoctonia solani, Pythium arrhenomannes, Pythium gramicola, Pythium aphanidermatum, High Plains Virus, European Wheat Striate Virus:

CANOLA: Albugo candida, Alternaria brassicae, Leptosharia maculans, Rhizoctonia solani, Sclerotinia sclerotiorum, Mycospaerella brassiccola, Pythium ultimum, Peronospora parasitica, Fusarium roseum, Alternaria alternata:

SUNFLOWER: Plasmophora halstedii, Scherotinia sclerotiorum, Aster Yellows, Septoria helianthi, Phomopsis helianthi, Alternaria helianthi, Alternaria zinniae, Botrytis cinera, Phoma macdonaldii, Macrophomina phaseolina, Erysiphe cichoracearum, Phizopus oryzae, Rhizopus arrhizus, Rhizopus stolonifer, Puccinia helianthi, Verticillium Dahliae, Erwinia carotovorum p.v. carotovora, Cephalosporium acremonium, Phytophthora cryptogea, Albugo tragopogonis: etc.

SORGHUM: Exserohilum turcicum, Colletotrichum graminicola (Glomerella graminicola), Cercospora sorghi, Gloeocercospora sorghi, Ascochyta sorghi, Pseudomonas syringae p.v. syringae, Xanthomonas campestris p.v. holcicola, Pseudomonas andropogonis, Puccinia purpurea, Macrophomina phaseolina, Periconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari Sporisorium relianum (Sphacelotheca reliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium Oxysporum, Pythium arrhenomanes, Pythium graminicola, etc.

ALFALFA: Clavibater michiganensis subsp. Insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Rhizoctonia solani, Uromyces striatus, Colletotrichum trifolii race 1 and race 2, Leptosphaerulina briosiana, Stemphylium botryosum, Stagonospora meliloti, Sclerotinia trifoliorum, Alfalfa Mosaic Virus, Verticillium albo-atrum, Xanthomonas campestris p.v. alfalfae, Aphanomyces euteiches, Stemphylium herbarum, Stemphylium alfalfae.

### VI. Controlling Gene Expression in Transgenic Plants

The invention further relates to transformed cells comprising the nucleic acid molecules, transformed plants, seeds, and plant parts, and methods of modifying phenotypic traits of interest by altering the expression of the genes of the invention.

### A. Modification of Coding Sequences and Adjacent Sequences

The transgenic expression in plants of genes derived from heterologous sources may involve the modification of those genes to achieve and optimize their expression in plants. In particular, bacterial ORFs which encode separate enzymes but which are encoded by the same transcript in the native microbe are best expressed in plants on separate transcripts. To achieve this, each microbial ORF is isolated individually and cloned within a cassette which provides a plant promoter sequence at the 5′ end of the ORF and a plant transcriptional terminator at the 3′ end of the ORF. The isolated ORF sequence preferably includes the initiating ATG codon and the terminating STOP codon but may include additional sequence beyond the initiating ATG and the STOP codon. In addition, the ORF may be truncated, but still retain the required activity; for particularly long ORFs, truncated versions which retain activity may be preferable for expression in transgenic organisms. By “plant promoter” and “plant transcriptional terminator” it is intended to mean promoters and transcriptional terminators which operate within plant cells. This includes promoters and transcription terminators which may be derived from non-plant sources such as viruses (an example is the Cauliflower Mosaic Virus).

In some cases, modification to the ORF coding sequences and adjacent sequence is not required. It is sufficient to isolate a fragment containing the ORF of interest and to insert it downstream of a plant promoter. For example, Gaffney et al. (Science 261: 754-756 (1993)) have expressed the Pseudomonas nahG gene in transgenic plants under the control of the CaMV 35S promoter and the CaMV tml terminator successfully without modification of the coding sequence and with nucleotides of the Pseudomonas gene upstream of the ATG still attached, and nucleotides downstream of the STOP codon still attached to the nahG ORF. Preferably as little adjacent microbial sequence should be left attached upstream of the ATG and downstream of the STOP codon. In practice, such construction may depend on the availability of restriction sites.

In other cases, the expression of genes derived from microbial sources may provide problems in expression. These problems have been well characterized in the art and are particularly common with genes derived from certain sources such as Bacillus. These problems may apply to the nucleotide sequence of this invention and the modification of these genes can be undertaken using techniques now well known in the art. The following problems may be encountered:

1. Codon Usage.

The preferred codon usage in plants differs from the preferred codon usage in certain microorganisms. Comparison of the usage of codons within a cloned microbial ORF to usage in plant genes (and in particular genes from the target plant) will enable an identification of the codons within the ORF which should preferably be changed. Typically plant evolution has tended towards a strong preference of the nucleotides C and G in the third base position of monocotyledons, whereas dicotyledons often use the nucleotides A or T at this position. By modifying a gene to incorporate preferred codon usage for a particular target transgenic species, many of the problems described below for GC/AT content and illegitimate splicing will be overcome.

2. GC/AT Content.

Plant genes typically have a GC content of more than 35%. ORF sequences which are rich in A and T nucleotides can cause several problems in plants. Firstly, motifs of ATTTA are believed to cause destabilization of messages and are found at the 3′ end of many short-lived mRNAs. Secondly, the occurrence of polyadenylation signals such as AATAAA at inappropriate positions within the message is believed to cause premature truncation of transcription. In addition, monocotyledons may recognize AT-rich sequences as splice sites (see below).

3. Sequences Adjacent to the Initiating Methionine.

Plants differ from microorganisms in that their messages do not possess a defined ribosome binding site. Rather, it is believed that ribosomes attach to the 5′ end of the message and scan for the first available ATG at which to start translation. Nevertheless, it is believed that there is a preference for certain nucleotides adjacent to the ATG and that expression of microbial genes can be enhanced by the inclusion of a eukaryotic consensus translation initiator at the ATG. Clontech (1993/1994 catalog, page 210, incorporated herein by reference) have suggested one sequence as a consensus translation initiator for the expression of the E. coli uidA gene in plants. Further, Joshi (N.A.R. 15: 6643-6653 (1987), incorporated herein by reference) has compared many plant sequences adjacent to the ATG and suggests another consensus sequence. In situations where difficulties are encountered in the expression of microbial ORFs in plants, inclusion of one of these sequences at the initiating ATG may improve translation. In such cases the last three nucleotides of the consensus may not be appropriate for inclusion in the modified sequence due to their modification of the second AA residue. Preferred sequences adjacent to the initiating methionine may differ between different plant species. A survey of 14 maize genes located in the GenBank database provided the following results:

Position Before the Initiating ATG in 14 Maize Genes:

| | −10 | −9 | −8 | −7 | −6 | −5 | −4 | −3 | −2 | −1 |
|---|---|---|---|---|---|---|---|---|---|---|
| C | 3 | 8 | 4 | 6 | 2 | 5 | 6 | 0 | 10 | 7 |
| T | 3 | 0 | 3 | 4 | 3 | 2 | 1 | 1 | 1 | 0 |
| A | 2 | 3 | 1 | 4 | 3 | 2 | 3 | 7 | 2 | 3 |
| G | 6 | 3 | 6 | 0 | 6 | 5 | 4 | 6 | 1 | 5 |

This analysis can be done for the desired plant species into which the nucleotide sequence is being incorporated, and the sequence adjacent to the ATG modified to incorporate the preferred nucleotides.

4. Removal of Illegitimate Splice Sites.

Genes cloned from non-plant sources and not optimized for expression in plants may also contain motifs which may be recognized in plants as 5′ or 3′ splice sites, and be cleaved, thus generating truncated or deleted messages. These sites can be removed using the techniques well known in the art.

Techniques for the modification of coding sequences and adjacent sequences are well known in the art. In cases where the initial expression of a microbial ORF is low and it is deemed appropriate to make alterations to the sequence as described above, then the construction of synthetic genes can be accomplished according to methods well known in the art. These are, for example, described in the published patent disclosures EP 0 385 962 (to Monsanto), EP 0 359 472 (to Lubrizol) and WO 93/07278 (to Ciba-Geigy), all of which are incorporated herein by reference. In most cases it is preferable to assay the expression of gene constructions using transient assay protocols (which are well known in the art) prior to their transfer to transgenic plants.

### B. Construction of Plant Expression Cassettes

Coding sequences intended for expression in transgenic plants are first assembled in expression cassettes behind a suitable promoter expressible in plants. The expression cassettes may also comprise any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors described below. The following is a description of various components of typical expression cassettes.

1. Promoters

The selection of the promoter used in expression cassettes will determine the spatial and temporal expression pattern of the transgene in the transgenic plant. Selected promoters will express transgenes in specific cell types (such as leaf epidermal cells, mesophyll cells, root cortex cells) or in specific tissues or organs (roots, leaves or flowers, for example) and the selection will reflect the desired location of accumulation of the gene product. Alternatively, the selected promoter may drive expression of the gene under various inducing conditions. Promoters vary in their strength, i.e., ability to promote transcription. Depending upon the host cell system utilized, any one of a number of suitable promoters can be used, including the gene's native promoter. The following are non-limiting examples of promoters that may be used in expression cassettes.

a. Constitutive Expression, the Ubiquitin Promoter:

Ubiquitin is a gene product known to accumulate in many cell types and its promoter has been cloned from several species for use in transgenic plants (e.g. sunflower—Binet et al. Plant Science 79: 87-94 (1991); maize—Christensen et al. Plant Molec. Biol. 12: 619-632 (1989); and Arabidopsis—Callis et al., J. Biol. Chem. 265: 12486-12493 (1990) and Norris et al., Plant Mol. Biol. 21: 895-906 (1993)). The maize ubiquitin promoter has been developed in transgenic monocot systems and its sequence and vectors constructed for monocot transformation are disclosed in the patent publication EP 0 342 926 (to Lubrizol) which is herein incorporated by reference. Taylor et al. (Plant Cell Rep. 12: 491495 (1993)) describe a vector (pAHC25) that comprises the maize ubiquitin promoter and first intron and its high activity in cell suspensions of numerous monocotyledons when introduced via microprojectile bombardment. The Arabidopsis ubiquitin promoter is ideal for use with the nucleotide sequences of the present invention. The ubiquitin promoter is suitable for gene expression in transgenic plants, both monocotyledons and dicotyledons. Suitable vectors are derivatives of pAHC25 or any of the transformation vectors described in this application, modified by the introduction of the appropriate ubiquitin promoter and/or intron sequences.

b. Constitutive Expression, the CaMV 35S Promoter:

Construction of the plasmid pCGN1761 is described in the published patent application EP 0 392 225 (Example 23), which is hereby incorporated by reference. pCGN1761 contains the “double” CaMV 35S promoter and the tml transcriptional terminator with a unique EcoRI site between the promoter and the terminator and has a pUC-type backbone. A derivative of pCGN1761 is constructed which has a modified polylinker which includes Notl and XhoI sites in addition to the existing EcoRI site. This derivative is designated pCGN1761ENX. pCGN1761ENX is useful for the cloning of cDNA sequences or coding sequences (including microbial ORF sequences) within its polylinker for the purpose of their expression under the control of the 35S promoter in transgenic plants. The entire 35S promoter-coding sequence-tml terminator cassette of such a construction can be excised by HindIII, SphI, SalI, and Xbal sites 5′ to the promoter and Xbal, BamHI and BglI sites 3′) to the terminator for transfer to transformation vectors such as those described below. Furthermore, the double 35S promoter fragment can be removed by 5′ excision with HindIII, SphI, SalI, Xbal, or Pstl, and 3′ excision with any of the polylinker restriction sites (EcoRI, Notl or XhoI) for replacement with another promoter. If desired, modifications around the cloning sites can be made by the introduction of sequences that may enhance translation. This is particularly useful when overexpression is desired. For example, pCGN1761ENX may be modified by optimization of the translational initiation site as described in Example 37 of U.S. Pat. No. 5,639,949, incorporated herein by reference.

c. Constitutive Expression, the Actin Promoter:

Several isoforms of actin are known to be expressed in most cell types and consequently the actin promoter is a good choice for a constitutive promoter. In particular, the promoter from the rice Actl gene has been cloned and characterized (McElroy et al. Plant Cell 2: 163-171 (1990)). A 1.3 kb fragment of the promoter was found to contain all the regulatory elements required for expression in rice protoplasts. Furthermore, numerous expression vectors based on the Actl promoter have been constructed specifically for use in monocotyledons (McElroy et al. Mol. Gen. Genet. 231: 150-160 (1991)). These incorporate the Actl-intron 1, Adhl 5′ flanking sequence and Adhl-intron 1 (from the maize alcohol dehydrogenase gene) and sequence from the CaMV 35S promoter. Vectors showing highest expression were fusions of 35S and Actl intron or the Actl 5′ flanking sequence and the Actl intron. Optimization of sequences around the initiating ATG (of the GUS reporter gene) also enhanced expression. The promoter expression cassettes described by McElroy et al. (Mol. Gen. Genet. 231: 150-160 (1991)) can be easily modified for gene expression and are particularly suitable for use in monocotyledonous hosts. For example, promoter-containing fragments is removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice Actl promoter with its first intron has also been found to direct high expression in cultured barley cells (Chibbar et al. Plant Cell Rep. 12: 506-509 (1993)).

d. Inducible Expression, PR-1 Promoters:

The double 35S promoter in pCGN1761 ENX may be replaced with any other promoter of choice that will result in suitably high expression levels. By way of example, one of the chemically regulatable promoters described in U.S. Pat. No. 5,614,395, such as the tobacco PR-1 promoter, may replace the double 35S promoter. Alternately, the Arabidopsis PR-1 promoter described in Lebel et al., Plant J. 16: 223-233 (1998) may be used. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemically/pathogen regulatable tobacco PR-1a promoter is cleaved from plasmid pCIB1004 (for construction, see example 21 of EP 0332 104, which is hereby incorporated by reference) and transferred to plasmid pCGN1761ENX (Uknes et al., Plant Cell 4: 645-656 (1992)). pCIB1004 is cleaved with Ncol and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761ENX from which the double 35S promoter has been removed. This is done by cleavage with XhoI and blunting with T4 polymerase, followed by cleavage with HindIII and isolation of the larger vector-terminator containing fragment into which the pCIB1004 promoter fragment is cloned. This generates a pCGN1761ENX derivative with the PR-1a promoter and the tml terminator and an intervening polylinker with unique EcoRI and Notl sites. The selected coding sequence can be inserted into this vector, and the fusion products (i.e. promoter-gene-terminator) can subsequently be transferred to any selected transformation vector, including those described infra. Various chemical regulators may be employed to induce expression of the selected coding sequence in the plants transformed according to the present invention, including the benzothiadiazole, isonicotinic acid, and salicylic acid compounds disclosed in U.S. Pat. Nos. 5,523,311 and 5,614,395.

e. Inducible Expression, an Ethanol-Inducible Promoter:

A promoter inducible by certain alcohols or ketones, such as ethanol, may also be used to confer inducible expression of a coding sequence of the present invention. Such a promoter is for example the alcA gene promoter from Aspergillus nidulans (Caddick et al. (1998) Nat. Biotechnol 16:177-180). In A. nidulans, the alcA gene encodes alcohol dehydrogenase 1, the expression of which is regulated by the AlcR transcription factors in presence of the chemical inducer. For the purposes of the present invention, the CAT coding sequences in plasmid palcA:CAT comprising a alcA gene promoter sequence fused to a minimal 35S promoter (Caddick et al. (1998) Nat. Biotechnol 16:177-180) are replaced by a coding sequence of the present invention to form an expression cassette having the coding sequence under the control of the alcA gene promoter. This is carried out using methods well known in the art.

f. Inducible Expression, a Glucocorticoid-lnducible Promoter:

Induction of expression of a nucleic acid sequence of the present invention using systems based on steroid hormones is also contemplated. For example, a glucocorticoid-mediated induction system is used (Aoyama and Chua (1997) The Plant Journal 11: 605-612) and gene expression is induced by application of a glucocorticoid, for example a synthetic glucocorticoid, preferably dexamethasone, preferably at a concentration ranging from 0.1 mM to 1 mM, more preferably from 10 mM to 100 mM. For the purposes of the present invention, the luciferase gene sequences are replaced by a nucleic acid sequence of the invention to form an expression cassette having a nucleic acid sequence of the invention under the control of six copies of the GAL4 upstream activating sequences fused to the 35S minimal promoter. This is carried out using methods well known in the art. The trans-acting factor comprises the GAL4 DNA-binding domain (Keegan et al. (1986) Science 231: 699-704) fused to the transactivating domain of the herpes viral protein VP16 (Triezenberg et al. (1988) Genes Devel. 2: 718-729) fused to the hormone-binding domain of the rat glucocorticoid receptor (Picard et al. (1988) Cell 54: 1073-1080). The expression of the fusion protein is controlled by any promoter suitable for expression in plants known in the art or described here. This expression cassette is also comprised in the plant comprising a nucleic acid sequence of the invention fused to the 6×GAL4/minimal promoter. Thus, tissue- or organ-specificity of the fusion protein is achieved leading to inducible tissue- or organ-specificity of the insecticidal toxin.

g. Root Specific Expression:

Another pattern of gene expression is root expression. A suitable root promoter is the promoter of the maize metallothionein-like (MTL) gene described by de Framond (FEBS 290: 103-106 (1991)) and also in U.S. Pat. No. 5,466,785, incorporated herein by reference. This “MTL” promoter is transferred to a suitable vector such as pCGN1761ENX for the insertion of a selected gene and subsequent transfer of the entire promoter-gene-terminator cassette to a transformation vector of interest.

h. Wound-Inducible Promoters:

Wound-inducible promoters may also be suitable for gene expression. Numerous such promoters have been described (e.g. Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), Warner et al. Plant J. 3: 191-201 (1993)) and all are suitable for use with the instant invention. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA which is wound induced and which can be used to isolate the cognate promoter using standard techniques. Similar, Firek et al. and Warner et al. have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to this invention, and used to express these genes at the sites of plant wounding.

i. Pith-Preferred Expression:

Patent Application WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the 35S promoter and be used to drive the expression of a foreign gene in a pith-preferred manner. In fact, fragments containing the pith-preferred promoter or parts thereof can be transferred to any vector and modified for utility in transgenic plants.

j. Leaf-Specific Expression:

A maize gene encoding phosphoenol carboxylase (PEPC) has been described by Hudspeth & Grula (Plant Molec Biol 12: 579-589 (1989)). Using standard molecular biological techniques the promoter for this gene can be used to drive the expression of any gene in a leaf-specific manner in transgenic plants.

k. Pollen-Specific Expression:

WO 93/07278 describes the isolation of the maize calcium-dependent protein kinase (CDPK) gene which is expressed in pollen cells. The gene sequence and promoter extend up to 1400 bp from the start of transcription. Using standard molecular biological techniques, this promoter or parts thereof, can be transferred to a vector such as pCGN1761 where it can replace the 35S promoter and be used to drive the expression of a nucleic acid sequence of the invention in a pollen-specific manner.

2. Transcriptional Terminators

A variety of transcriptional terminators are available for use in expression cassettes. These are responsible for the termination of transcription beyond the transgene and correct mRNA polyadenylation. Appropriate transcriptional terminators are those that are known to function in plants and include the CaMV 35S terminator, the tml terminator, the nopaline synthase terminator and the pea rbcS E9 terminator. These can be used in both monocotyledons and dicotyledons. In addition, a gene's native transcription terminator may be used.

3. Sequences for the Enhancement or Regulation of Expression

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adhl gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., Genes Develop. 1: 1183-1200 (1987)). In the same experimental system, the intron from the maize bronze1 gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al. Nucl. Acids Res. 15: 8693-8711 (1987); Skuzeski et al. Plant Molec. Biol. 15: 65-79 (1990)). Other leader sequences known in the art include but are not limited to: picornavirus leaders, for example, EMCV leader (Encephalomyocarditis 5′ noncoding region) (Elroy-Stein, O., Fuerst, T. R., and Moss, B. PNAS USA 86:6126-6130 (1989)); potyvirus leaders, for example, TEV leader (Tobacco Etch Virus) (Allison et al., 1986); MDMV leader (Maize Dwarf Mosaic Virus); Virology 154:9-20); human immunoglobulin heavy-chain binding protein (BiP) leader, (Macejak, D. G., and Sarnow, P., Nature 353: 90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, pages 237-256 (1989); and Maize Chlorotic Mottle Virus leader (MCMV) (Lommel, S. A. et al., Virology 81:382-385 (1991). See also, Della-Cioppa et al., Plant Physiology 84:965-968 (1987).

In addition to incorporating one or more of the aforementioned elements into the 5′ regulatory region of a target expression cassette of the invention, other elements peculiar to the target expression cassette may also be incorporated. Such elements include but are not limited to a minimal promoter. By minimal promoter it is intended that the basal promoter elements are inactive or nearly so without upstream activation. Such a promoter has low background activity in plants when there is no transactivator present or when enhancer or response element binding sites are absent. One minimal promoter that is particularly useful for target genes in plants is the Bz1 minimal promoter, which is obtained from the bronze1 gene of maize. The Bz1 core promoter is obtained from the “myc” mutant Bz1-luciferase construct pBz1LucR98 via cleavage at the Nhel site located at −53 to −58. Roth et al., Plant Cell 3: 317 (1991). The derived Bz1 core promoter fragment thus extends from −53 to +227 and includes the Bz1 intron-1 in the 5′ untranslated region. Also useful for the invention is a minimal promoter created by use of a synthetic TATA element. The TATA element allows recognition of the promoter by RNA polymerase factors and confers a basal level of gene expression in the absence of activation (see generally, Mukumoto (1993) Plant Mol Biol 23: 995-1003; Green (2000) Trends Biochem Sci 25: 59-63)

4. Targeting of the Gene Product Within the Cell

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins which is cleaved during chloroplast import to yield the mature protein (e.g. Comai et al. J. Biol. Chem. 263: 15104-15109 (1988)). These signal sequences can be fused to heterologous gene products to effect the import of heterologous products into the chloroplast (van den Broeck, et al. Nature 313: 358-363 (1985)). DNA encoding for appropriate signal sequences can be isolated from the 5′ end of the cDNAs encoding the RUBISCO protein, the CAB protein, the EPSP synthase enzyme, the GS2 protein and many other proteins which are known to be chloroplast localized. See also, the section entitled “Expression With Chloroplast Targeting” in Example 37 of U.S. Pat. No. 5,639,949.

Other gene products are localized to other organelles such as the mitochondrion and the peroxisome (e.g. Unger et al. Plant Molec. Biol. 13: 411-418 (1989)). The cDNAs encoding these products can also be manipulated to effect the targeting of heterologous gene products to these organelles. Examples of such sequences are the nuclear-encoded ATPases and specific aspartate amino transferase isoforms for mitochondria. Targeting cellular protein bodies has been described by Rogers et al. (Proc. Natl. Acad. Sci. USA 82: 6512-6516 (1985)).

In addition, sequences have been characterized which cause the targeting of gene products to other cell compartments. Amino terminal sequences are responsible for targeting to the ER, the apoplast, and extracellular secretion from aleurone cells (Koehler & Ho, Plant Cell 2: 769-783 (1990)). Additionally, amino terminal sequences in conjunction with carboxy terminal sequences are responsible for vacuolar targeting of gene products (Shinshi et al. Plant Molec. Biol. 14: 357-368 (1990)).

By the fusion of the appropriate targeting sequences described above to transgene sequences of interest it is possible to direct the transgene product to any organelle or cell compartment. For chloroplast targeting, for example, the chloroplast signal sequence from the RUBISCO gene, the CAB gene, the EPSP synthase gene, or the GS2 gene is fused in frame to the amino terminal ATG of the transgene. The signal sequence selected should include the known cleavage site, and the fusion constructed should take into account any amino acids after the cleavage site which are required for cleavage. In some cases this requirement may be fulfilled by the addition of a small number of amino acids between the cleavage site and the transgene ATG or, alternatively, replacement of some amino acids within the transgene sequence. Fusions constructed for chloroplast import can be tested for efficacy of chloroplast uptake by in vitro translation of in vitro transcribed constructions followed by in vitro chloroplast uptake using techniques described by Bartlett et al. In: Edelmann et al. (Eds.) Methods in Chloroplast Molecular Biology, Elsevier pp 1081-1091 (1982) and Wasmann et al. Mol. Gen. Genet. 205: 446-453 (1986). These construction techniques are well known in the art and are equally applicable to mitochondria and peroxisomes.

The above-described mechanisms for cellular targeting can be utilized not only in conjunction with their cognate promoters, but also in conjunction with heterologous promoters so as to effect a specific cell-targeting goal under the transcriptional regulation of a promoter that has an expression pattern different to that of the promoter from which the targeting signal derives.

### C. Construction of Plant Transformation Vectors

Numerous transformation vectors available for plant transformation are known to those of ordinary skill in the plant transformation arts, and the genes pertinent to this invention can be used in conjunction with any such vectors. The selection of vector will depend upon the preferred transformation technique and the target species for transformation. For certain target species, different antibiotic or herbicide selection markers may be preferred. Selection markers used routinely in transformation include the nptll gene, which confers resistance to kanamycin and related antibiotics (Messing & Vierra. Gene 19: 259-268 (1982); Bevan et al., Nature 304:184-187 (1983)), the bar gene, which confers resistance to the herbicide phosphinothricin (White et al., Nucl. Acids Res 18: 1062 (1990), Spencer et al. Theor. Appl. Genet 79: 625-631 (1990)), the hph gene, which confers resistance to the antibiotic hygromycin (Blochinger & Diggelmann, Mol Cell Biol 4: 2929-2931), and the dhfr gene, which confers resistance to methatrexate (Bourouis et al., EMBO J. 2(7): 1099-1104 (1983)), the EPSPS gene, which confers resistance to glyphosate (U.S. Pat. Nos. 4,940,935 and 5,188,642), and the mannose-6-phosphate isomerase gene, which provides the ability to metabolize mannose (U.S. Pat. Nos. 5,767,378 and 5,994,629).

1. Vectors Suitable for Agrobacterium Transformation

Many vectors are available for transformation using Agrobacterium tumefaciens. These typically carry at least one T-DNA border sequence and include vectors such as pBIN19 (Bevan, Nucl. Acids Res. (1984)). Below, the construction of two typical vectors suitable for Agrobacterium transformation is described.

a. pCIB200 and pCIB2001:

The binary vectors pCIB200 and pCIB2001 are used for the construction of recombinant vectors for use with Agrobacterium and are constructed in the following manner. pTJS75kan is created by Narl digestion of pTJS75 (Schmidhauser & Helinski, J. Bacteriol. 164: 446-455 (1985)) allowing excision of the tetracycline-resistance gene, followed by insertion of an Accl fragment from pUC4K carrying an NPTII (Messing & Vierra, Gene 19: 259-268 (1982): Bevan et al., Nature 304: 184-187 (1983): McBride et al., Plant Molecular Biology 14: 266-276 (1990)). XhoI linkers are ligated to the EcoRV fragment of PCIB7 which contains the left and right T-DNA borders, a plant selectable nos/nptlI chimeric gene and the pUC polylinker (Rothstein et al., Gene 53: 153-161 (1987)), and the XhoI-digested fragment are cloned into SalI-digested pTJS75kan to create pCIB200 (see also EP 0 332 104, example 19). pCIB200 contains the following unique polylinker restriction sites: EcoRI, SstI, KpnI, BgmlI, Xbal, and SalI. pCIB2001 is a derivative of pCIB200 created by the insertion into the polylinker of additional restriction sites. Unique restriction sites in the polylinker of pCIB2001 are EcoRI, SstI, KpnI, BglII, Xbal, SalI, MluI, BclI, AvrlI, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

b. pCIB10 and Hygromycin Selection Derivatives Thereof:

The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et at (Gene 53: 153-161 (1987)). Various derivatives of pCIB10 are constructed which incorporate the gene for hygromycin B phosphotransferase described by Gritz et al. (Gene 25: 179-188 (1983)). These derivatives enable selection of transgenic plant cells on hygromycin only (pCIB743), or hygromycin and kanamycin (pCIB715, pCIB717).

2. Vectors Suitable for Non-Agrobacterium Transformation

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector depends largely on the preferred selection for the species being transformed. Below, the construction of typical vectors suitable for non-Agrobacterium transformation is described.

a. pCIB3064:

pCIB3064 is a pUC-derived vector suitable for direct gene transfer techniques in combination with selection by the herbicide basta (or phosphinothricin). The plasmid pCIB246 comprises the CaMV 35S promoter in operational fuson to the E. coli GUS gene and the CaMV 35S transcriptional terminator and is described in the PCT published application WO 93/07278. The 35S promoter of this vector contains two ATG sequences 5′ of the start site. These sites are mutated using standard PCR techniques in such a way as to remove the ATGs and generate the restriction sites SspI and PvulI. The new restriction sites are 96 and 37 bp away from the unique SalI site and 101 and 42 bp away from the actual start site. The resultant derivative of pCIB246 is designated pCIB3025. The GUS gene is then excised from pCIB3025 by digestion with SalI and SacI, the termini rendered blunt and religated to generate plasmid pCIB3060. The plasmid pJIT82 is obtained from the John Innes Centre, Norwich and the a 400 bp SmaI fragment containing the bar gene from Streptomyces vifidochromogenes is excised and inserted into the HpaI site of pCIB3060 (Thompson et al. EMBO J 6: 2519-2523 (1987)). This generated pCIB3064, which comprises the bar gene under the control of the CaMV 35S promoter and terminator for herbicide selection, a gene for ampicillin resistance (for selection in E. coli) and a polylinker with the unique sites SphI, Pstl, HindIII, and BamHI. This vector is suitable for the cloning of plant expression cassettes containing their own regulatory signals.

b. pSOG19 and pSOG35:

pSOG35 is a transformation vector that utilizes the E. coli gene dihydrofolate reductase (DFR) as a selectable marker conferring resistance to methotrexate. PCR is used to amplify the 35S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-Pstl fragment from pB1221 (Clontech) which comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry the pUC gene for ampicillin resistance and have HindIII, SphI, Pstl and EcoRI sites available for the cloning of foreign substances.

3. Vector Suitable for Chloroplast Transformation

For expression of a nucleotide sequence of the present invention in plant plastids, plastid transformation vector pPH143 (WO 97/32011, example 36) is used. The nucleotide sequence is inserted into pPH143 thereby replacing the PROTOX coding sequence. This vector is then used for plastid transformation and selection of transformants for spectinomycin resistance. Alternatively, the nucleotide sequence is inserted in pPH143 so that it replaces the aadH gene. In this case, transformants are selected for resistance to PROTOX inhibitors.

### D. Transformation

Once a nucleic acid sequence of the invention has been cloned into an expression system, it is transformed into a plant cell. The receptor and target expression cassettes of the present invention can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

1. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation mediated uptake, particle bombardment-mediated delivery, or microinjection. Examples of these techniques are described by Paszkowski et al., EMBO J 3: 2717-2722 (1984), Potrykus et al., Mol. Gen. Genet. 199: 169-177 (1985), Reich et al., Biotechnology 4: 1001-1004 (1986), and Klein et al., Nature 327: 70-73 (1987). In each case the transformed cells are regenerated to whole plants using standard techniques known in the art.

Agrobacterium-mediated transformation is a preferred technique for transformation of dicotyledons because of its high efficiency of transformation and its broad utility with many different species. Agrobacterium transformation typically involves the transfer of the binary vector carrying the foreign DNA of interest (e.g. pCIB200 or pCIB2001) to an appropriate Agrobacterium strain which may depend of the complement of vir genes carried by the host Agrobacterium strain either on a co-resident Ti plasmid or chromosomally (e.g. strain CIB542 for pCIB200 and pCIB2001 (Uknes et al. Plant Cell 5: 159-169 (1993)). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, Nucl. Acids Res. 16: 9877 (1988)).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050, 5,036,006, and 5,100,792 all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium or a bacteriophage, each containing DNA sought to be introduced) can also be propelled into plant cell tissue.

2. Transformation of Monocotyledons

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e. co-transformation) and both these techniques are suitable for use with this invention. Co-transformation may have the advantage of avoiding complete vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. Biotechnology 4: 1093-1096 (1986)).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al. (Plant Cell 2: 603-618 (1990)) and Fromm et al. (Biotechnology 8: 833-839 (1990)) have published techniques for transformation of A188-derived maize line using particle bombardment. Furthermore, WO 93/07278 and Koziel et al. (Biotechnology 11: 194-200 (1993)) describe techniques for the transformation of elite inbred lines of maize by particle bombardment. This technique utilizes immature maize embryos of 1.5-2.5 mm length excised from a maize ear 14-15 days after pollination and a PDS-1000He Biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. Plant Cell Rep 7: 379-384 (1988); Shimamoto et al. Nature 338: 274-277 (1989); Datta et al. Biotechnology 8: 736-740 (1990)). Both types are also routinely transformable using particle bombardment (Christou et al. Biotechnology 9: 957-962 (1991)). Furthermore, WO 93/21335 describes techniques for the transformation of rice via electroporation.

Patent Application EP 0 332 581 describes techniques for the generation, transformation and regeneration of Pooideae protoplasts. These techniques allow the transformation of Dactylis and wheat. Furthermore, wheat transformation has been described by Vasil et al. (Biotechnology 10: 667-674 (1992)) using particle bombardment into cells of type C long-term regenerable callus, and also by Vasil et al. (Biotechnology11:

1553-1558 (1993)) and Weeks et al. (Plant Physiol. 102: 1077-1084 (1993)) using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashiga & Skoog, Physiologia Plantarum 15: 473-497 (1962)) and 3 mg/l 2,4-D for induction of somatic embryos, which is allowed to proceed in the dark. On the chosen day of bombardment, embryos are removed from the induction medium and placed onto the osmoticum (i.e. induction medium with sucrose or maltose added at the desired concentration, typically 15%). The embryos are allowed to plasmolyze for 2-3 hours and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics®) helium device using a burst pressure of ˜1000 psi using a standard 80 mesh screen. After bombardment, the embryos are placed back into the dark to recover for about 24 hours (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Tranformation of monocotyledons using Agrobacterium has also been described. See, WO 94/00977 and U.S. Pat. No. 5,591,616, both of which are incorporated herein by reference. See also, Negrotto et al., Plant Cell Reports 19: 798-803 (2000), incorporated herein by reference.

For this example, rice (Oryza sativa) is used for generating transgenic plants. Various rice cultivars can be used (Hiei et al., 1994, Plant Journal 6:271-282; Dong et al., 1996, Molecular Breeding 2:267-276; Hiei et al., 1997, Plant Molecular Biology, 35:205-218). Also, the various media constituents described below may be either varied in quantity or substituted. Embryogenic responses are initiated and/or cultures are established from mature embryos by culturing on MS-CIM medium (MS basal salts, 4.3 g/liter; B5 vitamins (200×), 5 ml/liter; Sucrose, 30 g/liter; proline, 500 mg/liter; glutamine, 500 mg/liter; casein hydrolysate, 300 mg/liter; 2,4-D (1 mg/ml), 2 ml/liter; adjust pH to 5.8 with 1 N KOH; Phytagel, 3 g/liter). Either mature embryos at the initial stages of culture response or established culture lines are inoculated and co-cultivated with the Agrobacterium tumefaciens strain LBA4404 (Agrobacterium) containing the desired vector construction. Agrobacterium is cultured from glycerol stocks on solid YPC medium (100 mg/L spectinomycin and any other appropriate antibiotic) for ˜2 days at 28° C. Agrobacterium is re-suspended in liquid MS-CIM medium. The Agrobacterium culture is diluted to an OD600 of 0.2-0.3 and acetosyringone is added to a final concentration of 200 uM. Acetosyringone is added before mixing the solution with the rice cultures to induce Agrobacterium for DNA transfer to the plant cells. For inoculation, the plant cultures are immersed in the bacterial suspension. The liquid bacterial suspension is removed and the inoculated cultures are placed on co-cultivation medium and incubated at 22° C. for two days. The cultures are then transferred to MS-CIM medium with Ticarcillin (400 mg/liter) to inhibit the growth of Agrobacterium. For constructs utilizing the PMI selectable marker gene (Reed et al., In Vitro Cell. Dev. Biol.-Plant 37:127-132), cultures are transferred to selection medium containing Mannose as a carbohydrate source (MS with 2% Mannose, 300 mg/liter Ticarcillin) after 7 days, and cultured for 3-4 weeks in the dark. Resistant colonies are then transferred to regeneration induction medium (MS with no 2,4-D, 0.5 mg/liter IAA, 1 mg/liter zeatin, 200 mg/liter timentin 2% Mannose and 3% Sorbitol) and grown in the dark for 14 days. Proliferating colonies are then transferred to another round of regeneration induction media and moved to the light growth room. Regenerated shoots are transferred to GA7 containers with GA7-1 medium (MS with no hormones and 2% Sorbitol) for 2 weeks and then moved to the greenhouse when they are large enough and have adequate roots. Plants are transplanted to soil in the greenhouse (To generation) grown to maturity, and the T₁ seed is harvested.

3. Transformation of Plastids

Seeds of Nicotiana tabacum c.v. ‘Xanthienc’ are germinated seven per plate in a 1” circular array on T agar medium and bombarded 12-14 days after sowing with 1 μm tungsten particles (M10, Biorad, Hercules, CA) coated with DNA from plasmids pPH143 and pPH145 essentially as described (Svab, Z. and Maliga, P. (1993) PNAS 90, 913-917). Bombarded seedlings are incubated on T medium for two days after which leaves are excised and placed abaxial side up in bright light (350-500 μmol photons/m²/s) on plates of RMOP medium (Svab, Z., Hajdukiewicz, P. and Maliga, P. (1990) PNAS 87, 8526-8530) containing 500 μg/ml spectinomycin dihydrochloride (Sigma, St. Louis, Mo.). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook et al., (1989) Molecular Cloning: A Laboratory Manual, Cold Spring Harbor Laboratory, Cold Spring Harbor). BamHI/EcoRI-digested total cellular DNA (Mettler, I. J. (1987) Plant Mol Biol Reporter 5, 346349) is separated on 1% Tris-borate (TBE) agarose gels, transferred to nylon membranes (Amersham) and probed with ³²P-labeled random primed DNA sequences corresponding to a 0.7 kb BamHI/HindIII DNA fragment from pC8 containing a portion of the rps7/12plastid targeting sequence. Homoplasmic shoots are rooted aseptically on spectinomycin-containing MS/IBA medium (McBride, K. E. et al. (1994) PNAS 91, 7301-7305) and transferred to the greenhouse.

### V. Breeding and Seed Production

### A. Breeding

The plants obtained via tranformation with a nucleic acid sequence of the present invention can be any of a wide variety of plant species, including those of monocots and dicots; however, the plants used in the method of the invention are preferably selected from the list of agronomically important target crops set forth supra. The expression of a gene of the present invention in combination with other characteristics important for production and quality can be incorporated into plant lines through breeding. Breeding approaches and techniques are known in the art. See, for example, Welsh J. R., Fundamentals of Plant Genetics and Breeding, John Wiley & Sons, NY (1981); Crop Breeding, Wood D. R. (Ed.) American Society of Agronomy Madison, Wis. (1983); Mayo O., The Theory of Plant Breeding, Second Edition, Clarendon Press, Oxford (1987); Singh, D.P., Breeding for Resistance to Diseases and Insect Pests, Springer-Verlag, NY (1986); and Wricke and Weber, Quantitative Genetics and Selection Plant Breeding, Walter de Gruyter and Co., Berlin (1986).

The genetic properties engineered into the transgenic seeds and plants described above are passed on by sexual reproduction or vegetative growth and can thus be maintained and propagated in progeny plants. Generally said maintenance and propagation make use of known agricultural methods developed to fit specific purposes such as tilling, sowing or harvesting, Specialized processes such as hydroponics or greenhouse technologies can also be applied. As the growing crop is vulnerable to attack and damages caused by insects or infections as well as to competition by weed plants, measures are undertaken to control weeds, plant diseases, insects, nematodes, and other adverse conditions to improve yield. These include mechanical measures such a tillage of the soil or removal of weeds and infected plants, as well as the application of agrochemicals such as herbicides, fungicides, gametocides, nematicides, growth regulants, ripening agents and insecticides.

Use of the advantageous genetic properties of the transgenic plants and seeds according to the invention can further be made in plant breeding, which aims at the development of plants with improved properties such as tolerance of pests, herbicides, or stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants. Depending on the desired properties, different breeding measures are taken. The relevant techniques are well known in the art and include but are not limited to hybridization, inbreeding, backcross breeding, multiline breeding, variety blend, interspecific hybridization, aneuploid techniques, etc. Hybridization techniques also include the sterilization of plants to yield male or female sterile plants by mechanical, chemical, or biochemical means. Cross pollination of a male sterile plant with pollen of a different line assures that the genome of the male sterile but female fertile plant will uniformly obtain properties of both parental lines. Thus, the transgenic seeds and plants according to the invention can be used for the breeding of improved plant lines, that for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions.

### B. Seed Production

In seed production, germination quality and uniformity of seeds are essential product characteristics. As it is difficult to keep a crop free from other crop and weed seeds, to control seedborne diseases, and to produce seed with good germination, fairly extensive and well-defined seed production practices have been developed by seed producers, who are experienced in the art of growing, conditioning and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (TMTD®), methalaxyl (Apron®), and pirimiphos-methyl (Actellic®). If desired, these compounds are formulated together with further carriers, surfactants or application-promoting adjuvants customarily employed in the art of formulation to provide protection against damage caused by bacterial, fungal or animal pests. The protectant coatings may be applied by impregnating propagation material with a liquid formulation or by coating with a combined wet or dry formulation. Other methods of application are also possible such as treatment directed at the buds or the fruit.

VI. Alteration of Expression of Nucleic Acid Molecules

For example, the alteration in expression of the nucleic acid molecules of the present invention is achieved in one of the following ways:

A. “Sense” Suppression

Alteration of the expression of a nucleotide sequence of the present invention, preferably reduction of its expression, is obtained by “sense” suppression (referenced in e.g. Jorgensen et al. (1996) Plant Mol. Biol. 31, 957-973). In this case, the entirety or a portion of a nucleotide sequence of the present invention is comprised in a DNA molecule. The DNA molecule is preferably operatively linked to a promoter functional in a cell comprising the target gene, preferably a plant cell, and introduced into the cell, in which the nucleotide sequence is expressible. The nucleotide sequence is inserted in the DNA molecule in the “sense orientation”, meaning that the coding strand of the nucleotide sequence can be transcribed. In a preferred embodiment, the nucleotide sequence is fully translatable and all the genetic information comprised in the nucleotide sequence, or portion thereof, is translated into a polypeptide. In another preferred embodiment, the nucleotide sequence is partially translatable and a short peptide is translated. In a preferred embodiment, this is achieved by inserting at least one premature stop codon in the nucleotide sequence, which bring translation to a halt. In another more preferred embodiment, the nucleotide sequence is transcribed but no translation product is being made. This is usually achieved by removing the start codon, e.g. the “ATG”, of the polypeptide encoded by the nucleotide sequence. In a further preferred embodiment, the DNA molecule comprising the nucleotide sequence, or a portion thereof, is stably integrated in the genome of the plant cell. In another preferred embodiment, the DNA molecule comprising the nucleotide sequence, or a portion thereof, is comprised in an extrachromosomally replicating molecule.

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

B. “Anti-Sense” Suppression

In another preferred embodiment, the alteration of the expression of a nucleotide sequence of the present invention, preferably the reduction of its expression is obtained by “anti-sense” suppression. The entirety or a portion of a nucleotide sequence of the present invention is comprised in a DNA molecule. The DNA molecule is preferably operatively linked to a promoter functional in a plant cell, and introduced in a plant cell, in which the nucleotide sequence is expressible. The nucleotide sequence is inserted in the DNA molecule in the “anti-sense orientation”, meaning that the reverse complement (also called sometimes non-coding strand) of the nucleotide sequence can be transcribed. In a preferred embodiment, the DNA molecule comprising the nucleotide sequence, or a portion thereof, is stably integrated in the genome of the plant cell. In another preferred embodiment the DNA molecule comprising the nucleotide sequence, or a portion thereof, is comprised in an extrachromosomally replicating molecule. Several publications describing this approach are cited for further illustration (Green, P. J. et al., Ann. Rev. Biochem. 55:569-597 (1986); van der Krol, A. R. et al, Antisense Nuc. Acids & Proteins, pp. 125-141 (1991); Abel, P. P. et al., PNAS. USA 86:6949-6952 (1989); Ecker, J. R. et al., Proc. Natl. Acad. Sci. USA 83:5372-5376 (August 1986)).

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical. Antisense suppression of the RARI nucleic acid molecules of the invention is more specifically described below in Example 5.

C. Homologous Recombination

In another preferred embodiment, at least one genomic copy corresponding to a nucleotide sequence of the present invention is modified in the genome of the plant by homologous recombination as further illustrated in Paszkowski et al., EMBO Journal 7:4021-26 (1988). This technique uses the property of homologous sequences to recognize each other and to exchange nucleotide sequences between each by a process known in the art as homologous recombination. Homologous recombination can occur between the chromosomal copy of a nucleotide sequence in a cell and an incoming copy of the nucleotide sequence introduced in the cell by transformation. Specific modifications are thus accurately introduced in the chromosomal copy of the nucleotide sequence. In one embodiment, the regulatory elements of the nucleotide sequence of the present invention are modified. Such regulatory elements are easily obtainable by screening a genomic library using the nucleotide sequence of the present invention, or a portion thereof, as a probe. The existing regulatory elements are replaced by different regulatory elements, thus altering expression of the nucleotide sequence, or they are mutated or deleted, thus abolishing the expression of the nucleotide sequence. In another embodiment, the nucleotide sequence is modified by deletion of a part of the nucleotide sequence or the entire nucleotide sequence, or by mutation. Expression of a mutated polypeptide in a plant cell is also contemplated in the present invention. More recent refinements of this technique to disrupt endogenous plant genes have been described (Kempin et al., Nature 389:802-803 (1997) and Miao and Lam, Plant J., 7:359-365 (1995).

In another preferred embodiment, a mutation in the chromosomal copy of a nucleotide sequence is introduced by transforming a cell with a chimeric oligonucleotide composed of a contiguous stretch of RNA and DNA residues in a duplex conformation with double hairpin caps on the ends. An additional feature of the oligonucleotide is for example the presence of 2′-O-methylation at the RNA residues. The RNA/DNA sequence is designed to align with the sequence of a chromosomal copy of a nucleotide sequence of the present invention and to contain the desired nucleotide change. For example, this technique is further illustrated in U.S. Pat. No. 5,501,967 and Zhu et al. (1999) Proc. Natl. Acad. Sci. USA 96: 8768-8773.

D. Ribozymes

In a further embodiment, the RNA coding for a polypeptide of the present invention is cleaved by a catalytic RNA, or ribozyme, specific for such RNA. The ribozyme is expressed in transgenic plants and results in reduced amounts of RNA coding for the polypeptide of the present invention in plant cells, thus leading to reduced amounts of polypeptide accumulated in the cells. This method is further illustrated in U.S. Pat. No. 4,987,071.

E. Dominant-Negative Mutants

In another preferred embodiment, the activity of the polypeptide encoded by the nucleotide sequences of this invention is changed. This is achieved by expression of dominant negative mutants of the proteins in transgenic plants, leading to the loss of activity of the endogenous protein.

F. Aptamers

In a further embodiment, the activity of polypeptide of the present invention is inhibited by expressing in transgenic plants nucleic acid ligands, so-called aptamers, which specifically bind to the protein. Aptamers are preferentially obtained by the SELEX (Systematic Evolution of Ligands by EXponential Enrichment) method. In the SELEX method, a candidate mixture of single stranded nucleic acids having regions of randomized sequence is contacted with the protein and those nucleic acids having an increased affinity to the target are partitioned from the remainder of the candidate mixture. The partitioned nucleic acids are amplified to yield a ligand enriched mixture. After several iterations a nucleic acid with optimal affinity to the polypeptide is obtained and is used for expression in transgenic plants. This method is further illustrated in U.S. Pat. No. 5,270,163.

G. Zinc Finger Proteins

A zinc finger protein that binds a nucleotide sequence of the present invention or to its regulatory region is also used to alter expression of the nucleotide sequence. Preferably, transcription of the nucleotide sequence is reduced or increased. Zinc finger proteins are for example described in Beerli et al. (1998) PNAS PNAS 95:14628-14633., or in WO 95/19431, WO 98/54311, or WO 96/06166, all incorporated herein by reference in their entirety.

H. dsRNA

Alteration of the expression of a nucleotide sequence of the present invention is also obtained by dsRNA interference as described for example in WO 99/32619, WO 99/53050 or WO 99/61631, all incorporated herein by reference in their entirety. In another preferred embodiment, the alteration of the expression of a nucleotide sequence of the present invention, preferably the reduction of its expression, is obtained by double-stranded RNA (dsRNA) interference. The entirety or, preferably a portion of a nucleotide sequence of the present invention is comprised in a DNA molecule. The size of the DNA molecule is preferably from 100 to 1000 nucleotides or more; the optimal size to be determined empirically. Two copies of the identical DNA molecule are linked, separated by a spacer DNA molecule, such that the first and second copies are in opposite orientations. In the preferred embodiment, the first copy of the DNA molecule is in the reverse complement (also known as the non-coding strand) and the second copy is the coding strand; in the most preferred embodiment, the first copy is the coding strand, and the second copy is the reverse complement. The size of the spacer DNA molecule is preferably 200 to 10,000 nucleotides, more preferably 400 to 5000 nucleotides and most preferably 600 to 1500 nucleotides in length. The spacer is preferably a random piece of DNA, more preferably a random piece of DNA without homology to the target organism for dsRNA interference, and most preferably a functional intron which is effectively spliced by the target organism. The two copies of the DNA molecule separated by the spacer are operatively linked to a promoter functional in a plant cell, and introduced in a plant cell, in which the nucleotide sequence is expressible. In a preferred embodiment, the DNA molecule comprising the nucleotide sequence, or a portion thereof, is stably integrated in the genome of the plant cell.

In another preferred embodiment the DNA molecule comprising the nucleotide sequence, or a portion thereof, is comprised in an extrachromosomally replicating molecule. Several publications describing this approach are cited for further illustration (Waterhouse et al. (1998) PNAS 95:13959-13964; Chuang and Meyerowitz (2000) PNAS 97:49854990; Smith et al. (2000) Nature 407:319-320). Alteration of the expression of a nucleotide sequence by dsRNA interference is also described in, for example WO 99/32619, WO 99/53050 or WO 99/61631, all incorporated herein by reference in their entirety

In transgenic plants containing one of the DNA molecules described immediately above, the expression of the nucleotide sequence corresponding to the nucleotide sequence comprised in the DNA molecule is preferably reduced. Preferably, the nucleotide sequence in the DNA molecule is at least 70% identical to the nucleotide sequence the expression of which is reduced, more preferably it is at least 80% identical, yet more preferably at least 90% identical, yet more preferably at least 95% identical, yet more preferably at least 99% identical.

I. Insertion of a DNA Molecule (Insertional Mutagenesis)

In another preferred embodiment, a DNA molecule is inserted into a chromosomal copy of a nucleotide sequence of the present invention, or into a regulatory region thereof. Preferably, such DNA molecule comprises a transposable element capable of transposition in a plant cell, such as e.g. Ac/Ds, Em/Spm, mutator. Alternatively, the DNA molecule comprises a T-DNA border of an Agrobacterium T-DNA. The DNA molecule may also comprise a recombinase or integrase recognition site which can be used to remove part of the DNA molecule from the chromosome of the plant cell. Methods of insertional mutagenesis using T-DNA, transposons, oligonucleotides or other methods known to those skilled in the art are also encompassed. Methods of using T-DNA and transposon for insertional mutagenesis are described in Winkler et al. (1989) Methods Mol. Biol. 82:129-136 and Martienssen (1998) PNAS 95:2021-2026, incorporated herein by reference in their entireties.

J. Deletion Mutagenesis

In yet another embodiment, a mutation of a nucleic acid molecule of the present invention is created in the genomic copy of the sequence in the cell or plant by deletion of a portion of the nucleotide sequence or regulator sequence. Methods of deletion mutagenesis are known to those skilled in the art. See, for example, Miao et al, (1995) Plant J. 7:359. In yet another embodiment, this deletion is created at random in a large population of plants by chemical mutagenesis or irradiation and a plant with a deletion in a gene of the present invention is isolated by forward or reverse genetics. Irradiation with fast neutrons or gamma rays is known to cause deletion mutations in plants (Silverstone et al, (1998) Plant Cell, 10:155-169; Bruggemann et al., (1996) Plant J., 10:755-760; Redei and Koncz in Methods in Arabidopsis Research, World Scientific Press (1992), pp. 16-82). Deletion mutations in a gene of the present invention can be recovered in a reverse genetics strategy using PCR with pooled sets of genomic DNAs as has been shown in C. elegans (Liu et al., (1999), Genome Research, 9:859-867.). A forward genetics strategy would involve mutagenesis of a line displaying PTGS followed by screening the M2 progeny for the absence of PTGS. Among these mutants would be expected to be some that disrupt a gene of the present invention. This could be assessed by Southern blot or PCR for a gene of the present invention with genomic DNA from these mutants.

K. Overexpression in a Plant Cell

In yet another preferred embodiment, a nucleotide sequence of the present invention encoding a polypeptide is over-expressed. Examples of nucleic acid molecules and expression cassettes for over-expression of a nucleic acid molecule of the present invention are described above. Methods known to those skilled in the art of over-expression of nucleic acid molecules are also encompassed by the present invention.

In a preferred embodiment, the expression of the nucleotide sequence of the present invention is altered in every cell of a plant. This is for example obtained though homologous recombination or by insertion in the chromosome. This is also for example obtained by expressing a sense or antisense RNA, zinc finger protein or ribozyme under the control of a promoter capable of expressing the sense or antisense RNA, zinc finger protein or ribozyme in every cell of a plant. Constitutive expression, inducible, tissue-specific or developmentally-regulated expression are also within the scope of the present invention and result in a constitutive, inducible, tissue-specific or developmentally-regulated alteration of the expression of a nucleotide sequence of the present invention in the plant cell. Constructs for expression of the sense or antisense RNA, zinc finger protein or ribozyme, or for over-expression of a nucleotide sequence of the present invention, are prepared and transformed into a plant cell according to the teachings of the present invention, e.g. as described infra.

### VII. Polypeptides

The present invention further relates to isolated polypeptides comprising the amino acid sequence of SEQ ID NO:2. In particular, isolated polypeptides comprising the amino acid sequence of SEQ ID NO:2, and variants having conservative amino acid modifications. One skilled in the art will recognize that individual substitutions, deletions or additions to a nucleic acid, peptide, polypeptide or protein sequence which alters, adds or deletes a single amino acid or a small percent of amino acids in the encoded sequence is a “conservative modification” where the modification results in the substitution of an amino acid with a chemically similar amino acid. Conservative modified variants provide similar biological activity as the unmodified polypeptide. Conservative substitution tables listing functionally similar amino acids are known in the art. See Crighton (1984) Proteins, W.H. Freeman and Company.

In a preferred embodiment, a polypeptide having substantial similarity to a polypeptide sequence listed in even numbered SEQ ID NO:2-64, or exon, domain, or feature thereof, is an allelic variant of the polypeptide sequence listed in even numbered SEQ ID NO:2-64. In another preferred embodiment, a polypeptide having substantial similarity to a polypeptide sequence listed in even numbered SEQ ID NO:2-64, or exon, domain, or feature thereof, is a naturally occurring variant of the polypeptide sequence listed in even numbered SEQ ID NO:2-64. In another preferred embodiment, a polypeptide having substantial similarity to a polypeptide sequence listed in even numbered SEQ ID NO:2-64, or exon, domain, or feature thereof, is a polymorphic variant of the polypeptide sequence listed in even numbered SEQ ID NO:2-64.

In an alternate preferred embodiment, the sequence having substantial similarity contains a deletion or insertion of at least one amino acid. In a more preferred embodiment, the deletion or insertion is of less than about ten amino acids. In a most preferred embodiment, the deletion or insertion is of less than about three amino acids.

In a preferred embodiment, the sequence having substantial similarity encodes a substitution in at least one amino acid.

Embodiments of the present invention also contemplate an isolated polypeptide containing a polypeptide sequence including:

(f) a polypeptide sequence listed in even numbered SEQ ID NO:2-64, or exon, domain, or feature thereof;

(g) a polypeptide sequence having substantial similarity to (a);

(h) a polypeptide sequence encoded by a nucleotide sequence identical to or having substantial similarity to a nucleotide sequence listed in odd numbered SEQ ID NO:1-63, or an exon, domain, or feature thereof, or a sequence complementary thereto;

(i) a polypeptide sequence encoded by a nucleotide sequence capable of hybridizing under medium stringency conditions to a nucleotide sequence listed in odd numbered SEQ ID NO:1-63, or to a sequence complementary thereto; and

(j) A Functional Fragment of (a), (b), (c) or (d).

In another preferred embodiment, the polypeptide having substantial similarity is an allelic variant of a polypeptide sequence listed in even numbered SEQ ID NO:2-64, or a fragment, domain, repeat, feature, or chimeras thereof. In another preferred embodiment, the isolated nucleic acid includes a plurality of regions from the polypeptide sequence encoded by a nucleotide sequence identical to or having substantial similarity to a nucleotide sequence listed in odd numbered SEQ ID NO:1-63, or fragment, domain, or feature thereof, or a sequence complementary thereto.

In another preferred embodiment, the polypeptide is a polypeptide sequence listed in odd numbered SEQ ID NO:2-64. In another preferred embodiment, the polypeptide is a functional fragment or domain. In yet another preferred embodiment, the polypeptide is a chimera, where the chimera may include functional protein domains, including domains, repeats, post-translational modification sites, or other features. In a more preferred embodiment, the polypeptide is a plant polypeptide. In a more preferred embodiment, the plant is a dicot. In a more preferred embodiment, the plant is a gymnosperm. In a more preferred embodiment, the plant is a monocot. In a more preferred embodiment, the monocot is a cereal. In a more preferred embodiment, the cereal may be, for example, maize, wheat, barley, oats, rye, millet, sorghum, triticale, secale, einkorn, spelt, emmer, teff, milo, flax, gramma grass, Tripsacum, and teosinte. In a most preferred embodiment, the cereal is rice.

In a preferred embodiment, the polypeptide is expressed in a specific location or tissue of a plant. In a more preferred embodiment, the location or tissue is for example, but not limited to, epidermis, vascular tissue, meristem, cambium, cortex or pith. In a most preferred embodiment, the location or tissue is leaf or sheath, root, flower, and developing ovule or seed. In a more preferred embodiment, the location or tissue may be, for example, epidermis, root, vascular tissue, meristem, cambium, cortex, pith, leaf, and flower. In a more preferred embodiment, the location or tissue is a seed.

In a preferred embodiment, the polypeptide sequence encoded by a nucleotide sequence having substantial similarity to a nucleotide sequence listed in odd numbered SEQ ID NO:1-63 or a fragment, domain, or feature thereof or a sequence complementary thereto, includes a deletion or insertion of at least one nucleotide. In a more preferred embodiment, the deletion or insertion is of less than about thirty nucleotides. In a most preferred embodiment, the deletion or insertion is of less than about five nucleotides.

In a preferred embodiment, the polypeptide sequence encoded by a nucleotide sequence having substantial similarity to a nucleotide sequence listed in odd numbered SEQ ID NO:1-63, or fragment, domain, or feature thereof or a sequence complementary thereto, includes a substitution of at least one codon. In a more preferred embodiment, the substitution is conservative.

In a preferred embodiment, the polypeptide sequences having substantial similarity to the polypeptide sequence listed in even numbered SEQ ID NO:2-64, or a fragment, domain, repeat, feature, or chimeras thereof includes a deletion or insertion of at least one amino acid.

The polypeptides of the invention, fragments thereof or variants thereof can comprise any number of contiguous amino acid residues from a polypeptide of the invention, wherein the number of residues is selected from the group of integers consisting of from 10 to the number of residues in a full-length polypeptide of the invention. Preferably, the portion or fragment of the polypeptide is a functional protein. The present invention includes active polypeptides having specific activity of at least 20%, 30%, or 40%, and preferably at least 505, 60%, or 70%, and most preferably at least 805, 90% or 95% that of the native (non-synthetic) endogenous polypeptide. Further, the substrate specificity (k_{cat}Kₘ) is optionally substantially similar to the native (non-synthetic), endogenous polypeptide. Typically the Kₘ will be at least 30%, 40%, or 50% of the native, endogenous polypeptide; and more preferably at least 605, 70%, 80%, or 90%. Methods of assaying and quantifying measures of activity and substrate specificity are well known to those of skill in the art.

The isolated polypeptides of the present invention will elicit production of an antibody specifically reactive to a polypeptide of the present invention when presented as an immunogen. Therefore, the polypeptides of the present invention can be employed as immunogens for constructing antibodies immunoreactive to a protein of the present invention for such purposes, but not limited to, immunoassays or protein purification techniques. Immunoassays for determining binding are well known to those of skill in the art such as, but not limited to, ELISAs or competitive immunoassays.

Embodiments of the present invention also relate to chimeric polypeptides encoded by the isolated nucleic acid molecules of the present disclosure including a chimeric polypeptide containing a polypeptide sequence encoded by an isolated nucleic acid containing a nucleotide sequence including:

(g) a nucleotide sequence listed in odd numbered SEQ ID NO:1-63, or exon, domain, or feature thereof;

(h) a nucleotide sequence having substantial similarity to (a);

(i) a nucleotide sequence capable of hybridizing to (a);

(j) a nucleotide sequence complementary to (a), (b) or (c); and

(k) a nucleotide sequence which is the reverse complement of (a), (b) or (c);

(l) or a functional fragment thereof.

A polypeptide containing a polypeptide sequence encoded by an isolated nucleic acid containing a nucleotide sequence, its complement, or its reverse complement, encoding a polypeptide including a polypeptide sequence including:

(g) a polypeptide sequence listed in even numbered SEQ ID NO:2-64, or a domain, repeat, feature, or chimeras thereof;

(h) a polypeptide sequence having substantial similarity to (a);

(i) a polypeptide sequence encoded by a nucleotide sequence identical to or having substantial similarity to a nucleotide sequence listed in odd numbered SEQ ID NO:1-63, or an exon, domain, or feature thereof, or a sequence complementary thereto;

(j) a polypeptide sequence encoded by a. nucleotide sequence capable of hybridizing under medium stringency conditions to a nucleotide sequence listed in odd numbered SEQ ID NO:1-63, or to a sequence complementary thereto; and

(k) a functional fragment of (a), (b), (c) or (d);

(l) or a functional fragment thereof.

The isolated nucleic acid molecules of the present invention are useful for expressing a polypeptide of the present invention in a recombinantly engineered cell such as a bacteria, yeast, insect, mammalian or plant cell. The cells produce the polypeptide in a non-natural condition (e.g. in quantity, composition, location and/or time) because they have been genetically altered to do so. Those skilled in the art are knowledgeable in the numerous expression systems available for expression of nucleic acids encoding a protein of the present invention, and will not be described in detail below.

Briefly, the expression of isolated nucleic acids encoding a polypeptide of the invention will typically be achieved, for example, by operably linking the nucleic acid or cDNA to a promoter (constitutive or regulatable) followed by incorporation into an expression vector. The vectors are suitable for replication and/or integration in either prokaryotes or eukaryotes. Commonly used expression vectors comprise transcription and translation terminators, initiation sequences and promoters for regulation of the expression of the nucleic acid molecule encoding the polypeptide. To obtain high levels of expression of the cloned nucleic acid molecule, it is desirable to use expression vectors comprising a strong promoter to direct transcription, a ribosome binding site for translation initiation, and a transcription/translation terminator. One skilled in the art will recognize that modifications may be made to the polypeptide of the present invention without diminishing its biological activity. Some modifications may be made to facilitate the cloning, expression or incorporation of the polypeptide of the invention into a fusion protein. Such modification are well known in the art and include, but are not limited to, a methionine added at the amino terminus to provide an initiation site, or additional amino acids (e.g. poly Histadine) placed on either terminus to create conveniently located purification sequences. Restricton sites or termination codons can also be introduced into the vector.

In a preferred embodiment, the expression vector includes one or more elements such as, for example, but not limited to, a promoter-enhancer sequence, a selection marker sequence, an origin of replication, an epitope-tag encoding sequence, or an affinity purification-tag encoding sequence. In a more preferred embodiment, the promoter-enhancer sequence may be, for example, the CaMV 35S promoter, the CaMV 19S promoter, the tobacco PR-la promoter, the ubiquitin promoter, and the phaseolin promoter. In another embodiment, the promoter is operable in plants, and more preferably, a constitutive or inducible promoter. In another preferred embodiment, the selection marker sequence encodes an antibiotic resistance gene. In another preferred embodiment, the epitope-tag sequence encodes V5, the peptide Phe-His-His-Thr-Thr, hemagglutinin, or glutathione-S-transferase. In another preferred embodiment the affinity purification-tag sequence encodes a polyamino acid sequence or a polypeptide. In a more preferred embodiment, the polyamino acid sequence is polyhistidine. In a more preferred embodiment, the polypeptide is chitin binding domain or glutathione-S-transferase. In a more preferred embodiment, the affinity purification-tag sequence comprises an intein encoding sequence.

Prokaryotic cells may be used a host cells, for example, but not limited to, Escherichia coli, and other microbial strains known to those in the art. Methods for expressing proteins in prokaryotic cells are well known to those in the art and can be found in many laboratory manuals such as Molecular Cloning: A Laboratory Manual, by J. Sambrook et al. (1989, Cold Spring Harbor Laboratory Press). A variety of promoters, ribosome binding sites, and operators to control expression are available to those skilled in the art, as are selectable markers such as antibiotic resistance genes. The type of vector chosen is to allow for optimal growth and expression in the selected cell type.

A variety of eukaryotic expression systems are available such as, but not limited to, yeast, insect cell lines, plant cells and mammalian cells. Expression and synthesis of heterologous proteins in yeast is well known (see Sherman et al., Methods in Yeast Genetics, Cold Spring Harbor Laboratory Press, 1982). Commonly used yeast strains widely used for production of eukaryotic proteins are Saccharomyces cerevisiae and Pichia pastoris, and vectors, strains and protocols for expression are available from commercial suppliers (e.g., Invitrogen).

Mammalian cell systems may be transfected with expression vectors for production of proteins. Many suitable host cell lines are available to those in the art, such as, but not limited to the HEK293, BHK21 and CHO cells lines. Expression vectors for these cells can include expression control sequences such as an origin of replication, a promoter, ( e.g., the CMV promoter, a HSV tk promoter or phosphoglycerate kinase (pgk) promoter), an enhancer, and protein processing sites such as ribosome binding sites, RNA splice sites, polyadenylation sites, and transcription terminator sequences. Other animal cell lines useful for the production of proteins are available commercially or from depositories such as the American Type Culture Collection.

Expression vectors for expressing proteins in insect cells are usually derived from the SF9 baculovirus or other viruses known in the art. A number of suitable insect cell lines are available including but not limited to, mosquito larvae, silkworm, armyworm, moth and Drosophila cell lines.

Methods of transfecting animal and lower eukaryotic cells are known. Numerous methods are used to make eukaryotic cells competent to introduce DNA such as but not limited to: calcium phosphate precipitation, fusion of the recipient cell with bacterial protoplasts containing the DNA, treatment of the recipient cells with liposomes containing the DNA, DEAE dextrin, electroporation, biolistics, and microinjection of the DNA directly into the cells. Tranfected cells are cultured using means well known in the art (see, Kuchler, R. J., Biochemical Methods in Cell Culture and Virology, Dowden, Hutchinson and Ross, Inc. 1997).

Once a polypeptide of the present invention is expressed it may be isolated and purified from the cells using methods known to those skilled in the art. The purification process may be monitored using Western blot techniques or radioimmunoassay or other standard immunoassay techniques. Protein purification techniques are commonly known and used by those in the art (see R. Scopes, Protein Purification: Principles and Practice, Springer-Verlag, New York 1982: Deutscher, Guide to Protein Purification, Academic Press (1990). Embodiments of the present invention provide a method of producing a recombinant protein in which the expression vector includes one or more elements including a promoter-enhancer sequence, a selection marker sequence, an origin of replication, an epitope-tag encoding sequence, and an affinity purification-tag encoding sequence. In one preferred embodiment, the nucleic acid construct includes an epitope-tag encoding sequence and the isolating step includes use of an antibody specific for the epitope-tag. In another preferred embodiment, the nucleic acid construct contains a polyamino acid encoding sequence and the isolating step includes use of a resin comprising a polyamino acid binding substance, preferably where the polyamino acid is polyhistidine and the polyamino binding resin is nickel-charged agarose resin. In yet another preferred embodiment, the nucleic acid construct contains a polypeptide encoding sequence and the isolating step includes the use of a resin containing a polypeptide binding substance, preferably where the polypeptide is a chitin binding domain and the resin contains chitin-sepharose.

The polypeptides of the present invention cam be synthesized using non-cellular synthetic methods known to those in the art. Techniques for solid phase synthesis are described by Barany and Mayfield, Solid-Phase Peptide Synthesis, pp. 3-284 in the Peptides: Analysis, Synthesis, Biology, Vol.2 Special Methods in Peptide Synthesis, Part A; Merrifield, et al., J. Am. Chem. Soc. 85:2149-56 (1963) and Stewart et al., Solid Phase Peptide Synthesis, 2^{nd} ed. Pierce Chem. Co., Rockford, Ill. (1984).

The present invention further provides a method for modifying (i.e. increasing or decreasing) the concentration or composition of the polypeptides of the invention in a plant or part thereof. Modification can be effected by increasing or decreasing the concentration and/or the composition (i.e. the ration of the polypeptides of the present invention) in a plant. The method comprised introducing into a plant cell with an expression cassette comprising a nucleic acid molecule of the present invention, or an nucleic acid encoding a RAR1 sequence as described above to obtain a transformed plant cell or tissue, culturing the transformed plant cell or tissue. The nucleic acid molecule can be under the regulation of a constitutive or inducible promoter. The method can further comprise inducing or repressing expression of a nucleic acid molecule of a sequence in the plant for a time sufficient to modify the concentration and/or composition in the plant or plant part.

A plant or plant part having modified expression of a nucleic acid molecule of the invention can be analyzed and selected using methods known to those skilled in the art such as, but not limited to, Southern blot, DNA sequencing, or PCR analysis using primers specific to the nucleic acid molecule and detecting amplicons produced therefrom.

In general, concentration or composition in increased or decreased by at least 5%, 10%, 20%, 30%, 40%, 50%, 60%, 70%, 80% or 90% relative to a native control plant, plant part or cell lacking the expression cassette.

The invention will be further described by reference to the following detailed examples. These examples are provided for purposes of illustration only, and are not intended to be limiting unless otherwise specified.

### EXAMPLES

Standard recombinant DNA and molecular cloning techniques used here are well known in the art and are described by J. Sambrook, et al., Molecular Cloning: A Laboratory Manual, 3d Ed., Cold Spring Harbor, N.Y.: Cold Spring Harbor Laboratory Press (2001); by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, New York, John Wiley and Sons Inc., (1988), Reiter, et al., Methods in Arabidopsis Research, World Scientific Press (1992), and Schultz et al., Plant Molecular Biology Manual, Kluwer Academic Publishers (1998).

### Example 1

### Identification of Nucleic Acid Molecules (cDNAs) from Rice

A list of over 250 genes from diverse plant species was compiled based on their adjudged potential to confer an agronomic trait in a crop plant. Genes were identified by the species name and the name of the gene. For example the Arabidopsis ethylene insensitive 2 (EIN2) gene is referred to as AtEIN2. The traits of interest included, but were not limited to, disease resistance, abiotic stress tolerance, enhanced nutritional value, yield, flowering time, and hormone response. These genes were conceptually translated, and the resultant predicted peptides encoded by these genes (defined as “queries”) were used to search for similar genes in the Syngenta/Myriad Genetics proprietary rice genomic database ( Myriad contigs version 8). Similarity searching was done using the TBlastN algorithm and both a Blosum 62 and a Pam70 matrix, with other parameters at default settings. Similar results were obtained with the two matrices. Searches were carried out on the TimeLogic DeCypher server at TMRI in San Diego, or locally, using the same database. High scoring genomic contig hits (P value <10⁻¹⁰) were designated as containing homologs; the highest scoring genomic contig was in most, but not all, cases designated as containing the rice ortholog. Orthologs were given an “Os” designation in front of their gene. Thus, the rice ortholog to the AtEIN2 query was designated OsEIN2. In exceptional cases, more than one putative rice ortholog to a single query was identified and further pursued (eg. OsEIN2, OsEIN2b, etc. . . ).

The rice ortholog thus identified resided in a contiguous stretch (“genomic contig”) of rice genomic DNA sequence. Various commercially available gene prediction algorithms (FGENESH, GenMark, GenMarkHMM, Genescan, GeneWise) were used to predict partial or full-length cDNAs of the rice orthologs. Predicted cDNAs (sequences in SBI predicted cDNAs.doc; conceptual translations in SBI predicted peptides) were used to search a database of assembled consensus sequences of sequenced rice cDNA library clones (“cDNA contigs”) using BlastN with default settings on the TimeLogic DeCypher server at TMRI in San Diego. cDNA contigs with identity or near-identity to the predicted cDNA were identified and DNA from the longest cDNA clone was obtained from stocks at Torrey Mesa Research Institute (“TMRI”) (San Diego). Additional cDNAs were obtained from designing primers to the full-length, or partial predicted cDNAs, amplifying the cDNAs by PCR from rice cDNA libraries or first-strand cDNA, and cloning them by conventional TA cloning or Gateway™ technology (using pCR2.1TOPO or pDONR201 vectors (Invitrogen), respectively). In either case, cDNA clones were sequenced to confirm their identity. These cDNAs constitute a subset of those included in the SBI predicted cDNAs and are physical DNA clones that have been validated by sequencing (Listing 3) and by comparing them against the original target rice ortholog sequence using similarity search algorithms as outlined above. BLASTX—compares a translation of the DNA sequencing in 6 frames to a protein database. The protein database used was GenBank non-reduntant peptides.

To assign function to these genes, the conceptual translations of the sequences in SBI Predicted cDNAs.doc and in SBI cDNAs.doc were used to search the SwissProt protein database and GenPept non-redundant conceptual translation database using the BlastP algorithm at default settings. Functional assignments to rice cDNAs, based on similarity for the corresponding peptides to proteins and genes of known function, are included in Tables 1-4 and the Examples below.

### Example 2

### Cloning and Sequence of Nucleic Acid Molecules from Rice

The longest clones corresponding to full-length contigs identified in the rice cDNA library were streaked out and DNA. was made from these cells. Alternatively, oligonucleotide primers to the 5′ and 3′ ends end of the predicted gene, and inclusive of the predicted start and stop codons, were used to PCR amplify the gene from rice first strand cDNA or the cDNA library. In some instances, these PCR primers included additional 5′ sequences for Gateway™ recombination-based cloning (invitrogen). PCR amplification was carried out using the HF Advantage II (Clonetech) or EXPAND (Roche) PCR kits according to the manufacturer's instructions. PCR products were cloned into pCR2.1-TOPO or pDONR201 according to the manufacturer's instructions (Invitrogen).

DNA preps for 24 independent clones was miniprepped following the manufacturer's instructions. DNA was subjected to sequencing analysis using the BigDyeT™ Terminator Kit according to manufacturer's instructions (ABI). Sequencing made use of primers designed to both strands of the predicted gene. Alternatively, the DNA was transformed into a strain with an active transposon, followed by sequencing of random independent colonies using primers from each of the transposon ends using the EZ::TN™ TET-1 Insertion kit (Epicentre). All sequencing data were analyzed and assembled using the Phred/Phrap/Consed software package (University of Washington) to an error ratio equal to or less than 10⁻⁴ at the consensus sequence level.

Consensus sequences were validated as being intact and the correct gene in several ways. The coding region was checked for being full length (predicted start and stop codons present) and uninterrupted (no internal stop codons). Alignment with the gene prediction and BLAST analysis was used to ascertain if this was the intended target gene. Most of the cDNA clones included 5′ and 3′ untranslated sequences, providing additional evidence that these were indeed full length. In some instances, silent or missense changes were observed (changes encoding the same or a different amino acid, respectively). These changes were most likely due to sequencing errors in the genomic reference sequence, although errors generated during PCR amplification could not be completely ruled out for clones generated using PCR.

Full-length cDNA clones or full-length open reading frames generated by PCR were cloned into custom-made binary destination vectors using Gateway™ recombination-based cloning per the manufacturer's instructions (Invitrogen). Alternatively, PCR products were cloned using conventional restriction enzyme-based cloning.

### Example 3

### Expression Vectors and Tranformation of Plants

Binary destination vectors for plant transformation consist of a binary backbone and a T-DNA portion. The binary backbone contains the sequences necessary for selection and growth in Escherichia coli DH-5□ (Invitrogen) and Agrobacterium tumefaciens LBA4404, including the bacterial spectinomycin antibiotic resistance aadA gene from E. coli transposon Tn7, origins of replication for E. coli (ColE1) and A. tumefaciens (VS1), and the A. tumefaciens virG gene. The T-DNA portion was flanked by the right and left border sequences and includes the Positech™ (Syngenta) plant selectable marker and a gene expression cassette which varies depending on the application. The Positech™ plant selectable marker in this instance consists of a rice ACT1 (actin) promoter driving expression of the PMI (phosphomannose isomerase) gene, followed by the cauliflower mosaic virus transcriptional terminator, and confers resistance to mannose.

The gene expression cassette portion of the binary destination vectors varies depending on the application. In general, the cassette consists of a promoter designed to express the gene in certain tissues of the plant, followed by cloning sites (in some cases interrupted by a segment of spacer DNA), and finally by the A. tumefaciens nos 3′ end transcriptional terminator. The promoters used are designed to express the gene of interest in specific target tissues (eg. endosperm: maize ADPgpp or □-zein, or barley □-thionin; eg. embryo: maize globulin or oleosin; eg. aleurone: barley a-amylase; eg. root: maize MSR1 and MRS3; eg. green tissue: maize PEPC) or constitutively (eg. maize UBI plus intron), depending on the gene of interest. The cloning site contains either unique restriction enzyme sites (for conventional cloning) and/or a Gateway™ recombination-based cloning cassette (Invitrogen), in either the forward or reverse orientation. In gene expression cassettes designed for double-stranded interfering RNA (dsRNAi) production, the cloning site is divided by a spacer region (eg. first intron of the rice SH1 gene), thus permitting the cloning of two gene fragments, in the forward and reverse orientations respectively. dsRNAi and antisense are two technologies available for silencing genes of interest.

Transformation of the nucleic acid molecules of the present invention into plants is performed using methods described above in the Detailed Description.

### Example 4

### Abiotic Stress Tolerance cDNAs and Analysis

The function of the protein encoded by each abiotic stress tolerance gene is determined from analysis of the open reading frame (ORF) in each cDNA as described in Example 1. Table 1 describes the assigned functions for the abiotic stress tolerance genes described in this application.

TABLE 1
Abiotic Stress Tolerance Genes

| | SEQ ID | Putative Function & Similar | | Homology Reference |
|---|---|---|---|---|
| Gene | Nos: | Genes | E value | and % Homology |
| ABF3 | 1-2 | drought tolerance | 1.00E−100 | Hobo et al., Proc. Natl. |
| | | TRAB1 from rice (Oryza | | Acad. Sci. U.S.A. 96 |
| | | sativa) - bZIP transcription | | (26), 15348-15353 |
| | | factor, interacts with VP1 | | (1999) |
| | | transcription factor, a key | | 203/326 (62%) |
| | | regulator of ABA | | |
| | | response genes. ABA | | |
| | | plant hormone mediates | | |
| | | drought tolerance | | |
| | | response. | | |
| ASK1 | 3-4 | drought & salt tolerance | 1.00E−162 | Yoon et al., Mol. Gen. |
| | | Protein kinase SPK-3 | | Genet. 255 (4), 359-371 |
| | | from soybean (Glycine | | (1997) |
| | | max); upregulated by | | 279/365 (76%) |
| | | drought and salt. | | |
| ES147 | 5-6 | salt tolerance | 0 | Galvez et al., Plant |
| | | E5I47 protein kinase from | | Physiol. 103, 257-265 |
| | | Lophopyrum elongatum; | | (1993) |
| | | upregulated by salt stress. | | 365/442 (82%) |
| FAD8 | 7-8 | cold tolerance | 0 | Horiguchi et al., |
| | | Fatty acid desaturase | | Physiol. Plantarum 96, |
| | | from wheat (Triticum | | 275-283 (1996) |
| | | aestivum); overexpression | | 300/353 (84%) |
| | | of Arabidopsis thaliana | | |
| | | gene confers cold | | |
| | | tolerance. | | |
| SAL1 | 9-10 | salt tolerance | 0 | Peng and Verma, J. |
| | | 3′(2′),5′-bisphosphate | | Biol. Chem. 270 (49), |
| | | nucleotidase from rice | | 29105-29110 (1995) |
| | | (Oryza sativa); | | 355/358 (99%) |
| | | overexpression enhances | | |
| | | salt tolerance | | |

The abiotic stress tolerance genes are evaluated for their effect(s) in transformed plants by testing the transgenic transformed plants or progeny plants as compared with non-transgenic plants. The plants are tested for their altered tolerance cold, drought, salt and heat using methods known to those skilled in the art, and examples of such assays are described below.

Cold tolerance is tested by placing transgenic and non-transgenic plants of the same age in growth chambers at 5° C., with a 12 hr light/dark cycle, at 80% humidity for 72 hours. The plants are observed for enhanced tolerance or sensitivity to cold.

Tolerance to salt is measured by using any salt tolerance assay known to those skilled in the art. In particular, the salt tolerance assay is performed essentially as follows:

Seeds from transformed plants and untransformed parental lines are sown on filter paper soaked with Yoshida solution placed in petri dishes. After 7 days of growth in the climate chamber seedlings (about 4 cm shoot length and 4 cm root length) will be exposed to salt stress as follows: seedlings will be transferred to 24 well plates supplemented with Yoshida solution (control) or Yoshida solution enriched with two different salt concentrations (as below). To ensure the contact of the entire root with the solution a piece of moistened absorbent cotton is placed on top of the root within the well flooded with the solution. Alternatively, the seeds may be grown in sand as a growth medium.

Control: Yoshida Solution Without Supplementary Salt

1) Yoshida solution enriched with NaCl 3.2 mg/l (48 mM)+CaCl₂ 3.6 mg/l (24 mM). This salt concentration evoked 50% growth reduction in shoot length.

2) Yoshida solution enriched with NaCl 6.4 mg/l (96 mM)+CaCl₂ 7.1 mg/l (48 mM).

Tissue is harvested at: 0, 6, 12, 24, and 36 hours. After exposure, the seedlings are separated into shoots and roots, or whole seedlings (whichever you prefer) and then immediately frozen in liquid nitrogen for RNA extraction and analysis. Total RNA extraction is performed using any known method in the art such as, an RNA extraction kit from Qiagen.

Alternatively, seedlings are observed for enhanced or decreased tolerance to grown under salt stress as compared to the untransformed parental variety or line. Samples are also taken for analysis of protein expression.

Yoshida Solutions

| | | Stock preparation | Culture solution | Final conc. | |
|---|---|---|---|---|---|
| Elem. | Chemical names | (g/10 L stock) | (ml stock/10 L solutions) | (mM (ppm)) | |
| (Macro) | | | | | |
| N | NH₄NO₃ | 914 | 12.5 | 1.43 | mM (114 pm) |
| P | NaH₂PO₄.2H₂O | 403 | 12.5 | 0.33 | mM (51 ppm) |
| K | K₂SO₄ | 714 | 12.5 | 0.51 | mM (89 ppm) |
| Ca | CaCl₂ | 886 | 12.5 | 1.0 | mM (111 ppm) |
| Mg | MgSO₄.7H₂O | 3240 | 12.5 | 1.6 | mM (394 ppm) |
| (Micro)* | | | | | |
| Mn | MnCl₂.4H₂O | 15-20 | | >0.01 | mM |
| Mo | (NH₄)₆.MO₇O₂₄.4H₂O | 1.5 | | 1.5 × 10⁻⁴ | mM |
| B | H₃BO₃ | 12 | | 0.02 | mM |
| Zn | ZnSO₄.7H₂O | 0.7 | | 3 × 10⁻⁴ | mM |
| Cu | CuSO₄.5H₂O | 0.31 | | 1.6 × 10⁻⁴ | mM |
| | Citric acid | 119 | 12.5 of mixture | 0.071 | mM |
| (Iron) | | | | | |
| Fe | Iron chelate | 160 | 12.5 | | |

*Mix microelements in 10 L distilled water in the order above.
pH, 5.8 adjust with H₂SO₄ if necessary

For drought tolerance, substitute 20% polyethylene glycol (PEG; MW 8000) for the salt solution.

### Example 5

### Disease Resistance Genes and Analysis

The function of the protein encoded by each disease resistance gene is determined from analysis of the open reading frame (ORF) in each cDNA as described in Example 1. Table 2 describes the assigned functions for the disease resistance genes described in this application.

TABLE 2
Disease Resistance Genes

| | | | | Homology |
|---|---|---|---|---|
| | SEQ | Putative Function | | Reference and |
| Gene | ID NO: | and Similar Genes | E value | % Homology |
| AOS2 | 11-12 | disease resistance | 1.00E−163 | Sivasankar |
| | | Allene oxide | | et al., |
| | | synthase from | | unpublished |
| | | tomato | | 271/418 (64%) |
| | | (Lycopersicon | | |
| | | esculentum); key | | |
| | | jasmonic acid | | |
| | | biosynthetic | | |
| | | enzyme gene. | | |
| BWMK1 | 13-14 | disease resistance | 0 | He et al., Mol. |
| | | MAP kinase from | | Plant Microbe |
| | | rice (Oryza sativa); | | Interact. |
| | | upregulated by | | 12: 1064-1073 |
| | | pathogen infection. | | (1999). |
| | | | | 505/506 (99%) |
| DDE1 | 15-16 | disease resistance | 1.00E−170 | Strassner et al., |
| | | 12- | | unpublished |
| | | oxophytodienoate | | 283/374 (75%) |
| | | reductase 3 from | | |
| | | tomato | | |
| | | (Lycopersicon | | |
| | | esculentum) - key | | |
| | | jasmonic acid | | |
| | | biosynthetic | | |
| | | enzyme gene. | | |
| ERF1 | 17-18 | disease resistance | 2.00E−36 | Solano et al., |
| | | Arabidopsis | | Genes Dev. |
| | | thaliana ethylene | | 12(23): |
| | | reponse factor | | 3703-3714 |
| | | transcription factor. | | (1998) |
| | | Ethylene plant | | 80/140 (57%) |
| | | hormone mediates | | |
| | | defense responses. | | |
| JISP6 | 19-20 | disease resistance | 1E−126 | Sasaki et al., |
| | | Bowman Birk | | unpublished |
| | | trypsin inhibitor | | 194/195 (99%) |
| | | from rice (Oryza | | 61/145 (42%) |
| | | sativa); induced by | | |
| | | jasmonic acid, plant | | |
| | | hormone mediating | | |
| | | plant defense | | |
| | | responses. | | |
| LOX1 | 21-22 | disease resistance | 0 | Peng et al., J. |
| | | Lipoxygenase from | | Biol. Chem. |
| | | rice (Oryza sativa) | | 269 (5), |
| | | induced by | | 3755-3761 |
| | | incompatible | | (1994) |
| | | pathogen infection. | | 906/924 (98%) |
| PIPLC1 | 23-24 | disease resistance | 0 | Song and |
| | | Phosphoinositide- | | Goodman, |
| | | specific | | unpublished |
| | | phospholipase C | | 562/599 (93%) |
| | | from rice (Oryza | | |
| | | sativa); upregulated | | |
| | | by pathogen | | |
| | | infection | | |

Assays for testing disease resistance to a variety of pathogens known to those skilled in the art are performed on transgenic plants and non-transgenic parental lines to determine alteration in disease resistance.

For example, but by no means limiting, such disease resistance assays are performed essentially as described below.

Rice Detached Leaf Assay for Bacterial Blight

This example describes the disease resistance assay of the rice gene transformed rice plants and control plants using the detached leaf assays for bacterial blight (Xanthomonas oryzae pv otyzae (Xoo or Xanthomonas; Mmixture of isolates XOO 698 and PXO 112)). Transgenic plants are also compared to resistance of rice plants treated with Bion™.

1. Rice seedlings are planted 1 seed per pot in 4 cm×4 cm pots with a mix of 50% peat and 50% John Innes Potting compost number 3 soil. Plants are checked twice daily and spot watered if soil appears dry on the surface. Plants are grown in a growth room (16 hour light cycle at a light intensity of 15000 μMol; 27° C. day 80% humidity; 20° C. night 90% humidity) until testing.

2. Plants treated with Bion™ (formulation type and strength e.g. azibenzolar-s-methly 800 g/kg wettable powder) are subjected to soil drench application is used 7 days prior to inoculating with bacteria. The 4 cm×4 cm pots have a volume of 40 ml with a headspace of 4 ml for the solution. Thus, applying a 600 ppm solution to the top of the plants in the pots will result in a 60 ppm treatment. A 600 ppm solution comprises 60 mg active ingredient in 100 ml water. Dilutions from this solution are made for treatments with lower concentrations. Prior to application of Bion™, the plants to be treated are placed in saucers 2 cm deep and are not watered 24-hours pre treatment. The Bion™ application is made by pipetting 40 ml of the chemical solution onto the surface of the soil in each pot. After 24 hours post treatment the saucers are removed and normal watering regime is restored

3. Xooanthomonas oryzae pv oryzae cultures for inoculation are produced from single isolate bacterial stocks (kept at 4° C. stored on) 2 days prior test date. Xooanthomonas oryzae pv oryzae bacterial cultures are grown in 500 ml nutrient broth. Bacteria are picked up on the tip of a sterile pipette and resuspended in 500 ml nutrient broth (recipe below). Cultures are incubated at 25° C. on a platform shaker (115 rpm) for between 1 and 4 days (typically flasks are used 2 days after introduction of the bacteria). Successful bacterial growth is indicated by the nutrient broth becoming opaque and a more intense yellow colour. Immediately before inoculation of leaf pieces flasks of Isolates XOO 698 (JH code K4214) and PXO 112 (JH code K4211) are mixed to produce a dual isolate inoculation.

4. For the Xanthomonas detached leaf assay, plants approximately 12 weeks old are used. A total of 15 leaf samples are cut from randomly selected plants of each line of interest (i.e. transgenic event or non transgenic germplasm), or each individual treatment (i.e. combination of line and chemical application). A leaf sample is a section of the leaf between 5 cm and 6 cm long, and the width of the leaf wide, and may include the tip of the leaf. Multiple leaf samples can be obtained from one leaf. Leaf samples are always taken from the youngest fully expanded leaf available on the plant.

Control lines and treatments are included consisting of leaf 30 leaf samples from 12-week-old non-transgenic (wildtype) plants of the same variety as that used in the generation of the transgenic events and 30 leaf samples from Bion™ treated wildtype plants (12-week-old). As some level of senescence regularly occurs in detached leaf assays further plates of leaves that are only inoculated with nutrient broth (i.e. uninoculated controls) are also prepared. These plates consist of 30 leaf samples from wfildtype plants, 30 leaf samples from Bion™ treated wildtype plants and 15 leaf samples from 2 transgenic lines selected at random. These control plates allow assessors to clearly establish the difference in appearance between disease symptomology and unrelated senescence in the leaf samples.

5. Leaf samples are placed adaxial side up onto petri dishes containing 1% tap water agar amended with 75 ppm benzimidazole. Leaf samples are fully randomised between plates with a maximum of 6 samples per plate.

6. Leaf samples are inoculated individually with a syringe by twice injecting approximately 0.1 ml of Xooanthomonas oryzae pv ofyzae (isolates XOO 698 mixed with PXO 112) bacterial culture solution into the tip end of the leaf sample (one injection either side of the vascular bundle). Inoculations are completed in a laminar flow hood to reduce contamination of the bacterial cultures. After inoculation the plates are placed into a controlled environment incubator with conditions set at 32° C. day, 25° C. night and a 16 hour light cycle. A maximum humidity of 90% is maintained in the cabinet throughout the plate incubation time.

Assessments of disease development and senescence levels are completed every 48 hours, for up to 10 days after inoculation. Assessments made after 2 days for “spontaneous suicide” and every 3 days for curl, health and levels of “ooze.” The key indication of disease establishment within the leaf sample was the presence of yellow bacterial exudates (ooze) at one or both cut ends of the leaf. Lower incidence of exudates is a measure of decreased disease development and, hence, enhanced disease resistance and in the line(s) of interest resistance within the leaf sample.

The data for the blight assay is collated from the levels of ooze that is observed on the leaf pieces. Ooze is a symptom of XZanthomonas infection documented as occurring in detached leaf assays and is based on a method described by G. L. Xie (Plant Disease 82:1007-1011 (September 1998). Ooze manifests as a yellow exudated that occurs at the cut ends of an infected leaf. Leaf pieces are scored differently depending on if the ooze is observed at the inoculated end only or if the ooze had developed through the leaf and is also present at the opposing end of the leaf to the end innocuated. It is assumed that if ooze is observed at both ends, the leaf sample is exhibiting no resistance to the disease. If the inoculated end only exhibited ooze, there is some indication that the leaf piece is showing some resistance to the disease. If no ooze is observed at either end of the leaf piece there is an indication of strong resistance to the disease. Leaf pieces are scored as having presense or absense of ooze at each end (no quantification of the amount of ooze present). Transgenic plants expressing disease resistance genes show improved disease resistance and produce less ooze than wildtype plants.

Recipes

Nutrient broth for Xanthornonas oryzae pv oiyzae inoculum production 6.5 g Nutrient Broth (Oxoid CM1) into 500 ml Demonized Water. Stir until fully dissolved (about 5 minutes). Autoclave at 121° C. for 20 minutes.

Rice Assay for Rice Blast Disease (Caused by Pyricularia Grisea; Also Known as Magnaporthe Grisea)

This example describes the bioassay for resistance of RAR1 transgenic rice to rice blast Pyricularia grisea (strain K4005).

1. Rice seedlings are planted 1 seed per pot in 4 cm×4 cm pots with a mix of 50% peat and 50% John Innes Potting compost number 3 soil. Plants are checked twice daily and spot watered if soil appears dry on the surface. Plants are grown in a growthroom (16 hour light cycle at a light intensity of 15000 μMol; 27° C. day 80% humidity; 20° C. night 90% humidity) until testing.

2. Plants treated with Bion, are treated using a drench application 7 days prior to inoculation. The 4 cm×4 cm diameter pots have a volume of 150 mis with a headspace of 15 mls for the solution. Thus, applying a 600 ppm solution to the top of the plants in the pots will result in a 60 ppm treatment. A 600 ppm solution is made up of 60 mg active ingredient in 100 ml water. Make dilutions from that solution for treatments with lower concentrations.

3. Pyricularia grisea inoculum is prepared from 5 day old single isolate stock plates (kept at 25° C. on rice leaf extract agar—recipe below) immediately before required for inoculation. 20 ml sterile deionised water is added to a plate of Pyricularia grisea, which is then rubbed with a small soft brush to encourage the spores into solution. The resulting spore and mycelium solution is then filtered through one layer of fine mesh muslin. Spores are counted in with using haemocytometer and the inoculum solution was diluted to produce a concentration of 200,000 spores/ml. The inoculum is used within one hour of production. It is recommended to allow 5 ml of inoculum per plate. Rice leaf extract agar for Pyricularia inoculum production: 45 g Czapek Dox Agar, 10 g Oxide Agar No.3, 1000 ml rice leaf extract. Extract 50 g of dried straw with 1000 ml of water at 100° C. for 1 hour. Autoclave at 121° C. for 20 minutes.

For the Pyricularia grisea detached leaf assay plants approximately 12 weeks old were used. A total of 15 leaf samples are cut from randomly selected plants of each line of interest (i.e. transgenic event or non transgenic germplasm), or each individual treatment (i.e. combination of line and chemical application). A leaf sample is a section of the leaf between 5 cm and 6 cm long, and the width of the leaf wide, and may include the tip of the leaf. Leaf pieces are placed so that both ends of the leaf were buried into the agar as this increases the green life of the leaf samples. Multiple leaf samples can be obtained from one leaf. Leaf samples are always taken from the youngest fully expanded leaf available on the plant. Control lines and treatments are included consisting of leaf 30 leaf samples from 12 week old non-transgenic (wildtype) plants of the same variety as that used in the generation of the transgenic events and 30 leaf samples from Bion™ treated wildtype plants (12 weeks old). As some level of senescence regularly occurs in detached leaf assays further plates of leaves that are inoculated with only sterile deionized water (i.e. uninoculated controls) were also prepared. These plates consisted of 30 leaf samples from wildtype plants, 30 leaf samples from Bion™ treated wildtype plants and 15 leaf samples from 2 transgenic lines selected at random. These control plates allow assessors to establish clearly the difference in appearance between disease symptomology and unrelated senescence in the leaf samples.

5. Leaf samples are placed adaxial surface upwards onto petri dishes containing 1% tap water agar amended with 75 ppm benzimidazole. Leaf samples are fully randomised .between plates with a maximum of 6 samples per plate.

6. Inoculum is sprayed onto the plates using a Devilbiss spray gun. Leaf pieces are sprayed to produce an equal coverage of droplets over the exposed leaf surface. The petri dish plate lids are replaced immediately and plates are incubated in a controlled environment cabinet for up to 8 days (conditions—14 hour light cycle; 24° C. day; 24° C. night constant 90% humidity).

7. Plates are assessed for disease development (expressed as a estimated % disease coverage) and senescence levels every 48 hours for up to 8 days.

Medium for Xanthomonas otyzae pv oryzae Storage-Wakimoto Media

The data from the rice blast assay show that the transgenic lines assayed showed less disease coverage than the wildtype Kaybonnet rice line, demonstrating enhanced disease resistance in these lines. The wildtype treated with Bion™ showed the expected effect of decreased disease coverage.

This data clearly demonstrates that overexpression of the disease resistance gene in transgenic plants enhances disease resistance.

### Example 6

### Grain Quality and Nutritional Composition Genes and Analysis

The function of the protein encoded by each grain quality or nutritional composition gene is determined from analysis of the open reading frame (ORF) in each cDNA as described in Example 1. Table 3 describes the assigned functions for these genes described in this application.

TABLE 3
Grain Quality and Nutritional Composition Genes

| | SEQ | Putative Function and | | Homology Reference and % |
|---|---|---|---|---|
| Gene | ID NO | Similar Gene | E value | Homology |
| BT1 | 25-26 | starch level | 8.00E−70 | Sullivan et al., Plant Cell 3 |
| | | Adenylate transporter | | (12), 1337-1348 (1991) |
| | | from maize (Zea mays) - | | 165/305 (54%) |
| | | brittle-1, key starch | | 53/165 (32%) |
| | | biosynthesis protein | | 29/90 (32%) |
| BTH1 | 27-28 | thiamin level | 1.00E−180 | Theologis et al., Nature 408 |
| | | Arabidopsis thaliana | | (6814), 816-820 (2000) |
| | | phosphomethylpyrimidine | | 328/499 (65%) |
| | | kinase - thiamin B vitamin | | |
| | | biosynthetic enzyme gene | | |
| DHPS1 | 29-30 | lysine level | 1.00E−139 | Suh et al., unpublished |
| | | Dihydrodipicolinate | | 308/336 (91%) |
| | | synthase from rice (Oryza | | |
| | | sativa) - key lysine | | |
| | | biosynthetic enzyme | | |
| FER | 31-32 | iron level | 1.00E−139 | Wu et al., unpublished |
| | | Ferritin iron storage | | 251/255 (98%) |
| | | protein from rice (Oryza | | |
| | | sativa). | | |
| FER1 | 33-34 | iron level | 1.00E−135 | Wu et al., unpublished |
| | | Ferritin iron storage | | 245/255 (96%) |
| | | protein from rice (Oryza | | |
| | | sativa). | | |
| GAMYB1 | 35-36 | enzyme activation | 0 | Gubler, F. et al. Plant Cell |
| | | Myb transcription factor | | Physiol. 38(3): 362-365 |
| | | from rice; barley ortholog | | (1997) |
| | | mediates activation of | | 478/555 (86%) |
| | | aleurone enzymes for | | |
| | | mobilization of endoperm | | |
| | | food stores for the | | |
| | | developing embryo. | | |
| GTMT | 37-38 | vitamin E level | 1.00E−120 | Shintani and DellaPenna, |
| | | Arabidopsis thaliana | | Science 282(5396): 2098-2100 |
| | | gamma-tocopherol | | (1998) |
| | | methyltransferase, key | | 215/331 (64%) |
| | | Vitamin E biosynthetic | | |
| | | enzyme. Overexpression | | |
| | | in Arabidopsis increases | | |
| | | Vitamin E levels. | | |
| ISPE | 39-40 | vitamin A level | 1.00E−154 | Lawrence et al., Plant Mol. |
| | | 4-diphosphocytidyl-2-C- | | Biol. 33 (3), 483-492 (1997) |
| | | methyl-D-erythritol kinase | | 258/336 (76%) |
| | | from tomato | | |
| | | (Lycopersicon | | |
| | | esculentum) - key Vitamin | | |
| | | A biosynthetic enzyme. | | |
| OHP1 | 41-42 | protein level | 0 | Nakase et al., Plant Mol. |
| | | bZIP transcription factor | | Biol. 33 (3), 513-522 (1997) |
| | | from rice (Oryza sativa); | | 423/425 (99%) |
| | | positive regulator of seed | | |
| | | storage protein genes. | | |

Assays for testing for a variety of grain quality or nutritional composition qualities known to those skilled in the art are performed on transgenic plants and non-transgenic parental lines to determine alteration in any such qualities.

For example, but by no means limiting, such quality assays are performed essentially as described below.

An assay to analyze alterations in vitamin E content or beta-carotene (pro-vitamin A) content are performed essentially as described using high performance liquid chromatography (HPLC) in Fraser et al. Plant J. (2000) 24:551-458.

Assays for alteration of iron content are performed essentially as described in Goto et al. (1999) Nat. Biotech. 17:282-286 or Kamachi et al. (1992) Plant Physiol. 99:1481-1486.

Assays for alteration of enhanced phosphorus uptake are performed by growing plants under phosphorus limiting conditions for their entire life cycle, and growth (final dry mass) and yield (seed size and weight) are measured essentially as described in Fohse et al. (1988) Plant Soil 110:101-109.

Assays for alterantion of thiamin levels are performed essentially as described in Esteve et al. (2001) J. Agric. Food Chem. 49:1450.

The malting assays below are used for analyzing alterations due to the presence of GAMYB1. Malt and bacterial beta glucanase and cellulase assay procedure (azo barley glucan method) from Megazyme, Megazyme Int'l Ireland Ltd., Bray, co. Wicklow, Ireland, www.megazyme.com and the Amylazyme- alpha- amylase assay procedure from Megazyme.

A preferred assay for amino acid content of seeds is as follows:

Prepare: 80% EtOH in 45° C. water bath; 80% EtOH at room temp.; hexane; sealable glass tubes; 2M NH₄OH; 0.02M HCl; ddH₂O; and Dowex columns.

This protocol has been used for extraction of amino acids from seeds at 4DAF to mature. All samples are stored at −30° C. until extraction. Seeds are separated from the siliques and counted. Approximately 600 seeds are required for the each sample. This protocol is for extraction from approximately 600 seeds. Amino acids are quantified according to amount per 100 seeds (total amount), NOT amount per mg seeds (concentration). We believe this is a more accurate approach.

Crude Extract Preparation:

Homogenise approximately 600 seeds in 1 ml 45° C. 80% EtOH

1. Split the sample into 2 eppendorf tubes

2. Rinse the homogeniser 4 times with 300 μl of 45° C. 80% EtOH, transfering each rinse evenly into the 2 eppen tubes

3. Centrifuge sample at 5,000 rpm for 10 min at 4° C.

4. Transfer sup to fresh eppen tubes on ice

5. Re-suspend pellets in 100 μl of 45° C. 80% EtOH (pellet wash)

6. Vortex to re-suspend and place on shaker for 5 min

7. Centrifuge sample at 5,000 rpm for 10 min at 4° C.

8. Transfer sup to the eppen tubes on ice

9. Supernatant fraction: Soluble amino acids fraction Pellet fraction: Protein-bound amino acids fraction

For the soluble amino acids fraction, go direct to Dowex Column Purification (step 23). For the protein-bound fraction, continue with Extraction of Protein-bound Amino Acids (step 12)

Extraction of Protein Bound Amino Acids

12. Evaporate excess EtOH by putting sample on 45° C. heat block for 3 min

13. Add 600 μl hexane and vortex on shaker for 30 min (in draft hood)

14. Centrifuge sample at 5,000 rpm for 10 min at 4° C.

15. Discard sup and resuspend pellet as much as possible in 1 ml 6M HCl

16. Transfer the 2 solutions/sample into 1 sealable glass tube

17. Seal the glass tube under vacuum and incubate at 110° C. for 18 hr to hydrolyze the protein

18. After samples have cooled to rt, break open glass tube and transfer to eppen tubes

19. Dry up samples in vacuum centrifuge (takes 3 to 4 hrs)

20. Add 500 μl of 45° C. 80% EtOH. Vortex to resuspend and place on shaker for 5 min

21. Centrifuge sample at 5,000 rpm for 10 min at 4° C.

Transfer sup to new eppen (solubilised protein-bound amino acids) and continue to Dowex Column Purification (step 23)

23. Prepare 200 μl Dowex H⁺ column

24. Flow through about 1 ml of rt 80% EtOH

25. Apply sample in 4 stages, allowing to flow through by gravity

26. Wash 3 times with 400 μl (2× column volume) of rt 80% EtOH (total=1.2 ml, 6× column volume)

27. Wash 3 times with 400 μl (2× column volume) of ddH₂O (total1 .2 ml, 6× column volume)

28. Elute sample into fresh eppen tubes over 4 stages with 250 μl of 2M NH₄OH (total=1.0 ml, 5× column volume)

29. Keep samples on ice—samples can be stored o/n at 4° C. if desired

30. Split the samples into 2 eppen tubes and dry sample in vacuum centrifuge with heater (approx. 2.5 hrs). Although sample can fit in 1 eppen, drying is quicker with the 2 smaller volumes

31. Add 40 μl ice-cold 0.02M HCl to each eppen

32. Vortex to re-suspend, place on a medium-speed shaker for 15 minutes

33. Combine the samples into one of the eppen tubes then rinse the empty eppen with another 30 μl and add to the sample

34. Transfer to millipore columns (yellow lid)

35. Centrifuge for 1 min at 7,000 rpm at 4° C.

36. Discard the filter column from the tube and seal with parafilm

37. Store samples in 4° C., can be stored for up to 3 months

For the soluble amino acid fraction samples, use undiluted for HPLC analysis. The protein-bound samples may also need to be diluted

The millipore columns we use are Ultrafre-MC 0.22 μm filter units that resemble an eppendorf tube. The samples are analyzed by HPLC, preferably an M analyzer L8800 from Hitachi.

See references, Inaba, K., Fujiwara, T., Hayashi, H., Chino, M., Komeda, Y. and Naito, S. (1994). Isolation of an Arabidopsis thaliana mutant mto1 that overaccumulates soluble methionine: temporal and spatial patterns of soluble methionine accumulation. Plant Physiol. 10 104: 881-887, and Kho, C. and De Lumen, B. O. (1988). Identification and isolation of methionine-cysteine rich protein in soybean seed. Plant Food for Human Nutrition. 38: 287-296.

### Example 7

### Enhanced Yield Genes and Analysis

The function of the protein encoded by each yield related gene is determined from analysis of the open reading frame (ORF) in each cDNA as described in Example 1. Table 4 describes the assigned functions for these genes described in this application.

TABLE 4
Enhanced Yield Genes

| | SEQ ID | Putative Function and | | Homology References |
|---|---|---|---|---|
| Gene | NO | Similar Gene | E value | and % Homology |
| FT | 43-44 | time to harvest | 1.00E−197 | Kardailsky et al., Science 286 |
| | | Arabidopsis thaliana | | (5446), 1962-1965 (1999) |
| | | flowering locus T (FT) | | 173/176 (98%) |
| | | gene; mutation results in | | |
| | | delay of flowering. | | |
| IDS1 | 45-46 | seed number | 1.00E−66 | Chuck et al., Genes Dev. 12 (8), |
| | | IDS1 indeterminate | | 1145-1154 (1998) |
| | | spikelet 1 from maize (Zea | | 135/214 (63%) |
| | | mays) - regulator of floral | | 24/42 (57%) |
| | | meristem determinacy | | |
| IDS2 | 47-48 | iron acquisition | 1.00E−115 | Mori and Nakanishi, unpublished |
| | | Iron siderophore | | 204/344 (59%) |
| | | biosynthetic enzyme gene | | |
| | | IDS3 from barley | | |
| | | (Hordeum vulgare); | | |
| | | enhances iron uptake by | | |
| | | roots. | | |
| IDS3 | 49-50 | iron acquisition | 1.00E−120 | Mori and Nakanishi, unpublished |
| | | Iron siderophore | | 204/315 (64%) |
| | | biosynthetic enzyme gene | | |
| | | IDS3 from barley | | |
| | | (Hordeum vulgare); | | |
| | | enhances iron uptake by | | |
| | | roots. | | |
| IFL1 | 51-42 | seed number | 0 | Zhong and Ye, Plant Cell 11 |
| | | Arabidopsis thaliana | | (11), 2139-2152 (1999) |
| | | homeodomain-leucine | | 576/840 (68%) |
| | | zipper transcription factor | | |
| | | IFL1 - positive regulator of | | |
| | | inflorescence branching | | |
| IRT1 | 53-54 | iron acquisition | 2.00E−97 | Eckhardt et al., Plant Mol. Biol. |
| | | Iron transporter 1 (IRT1) | | 45 (4), 437-448 (2001) |
| | | from tomato (Lycopersicon | | 186/338 (55%) |
| | | esculentum). | | |
| NAAT | 55-56 | iron acquisition | 1.00E−139 | Takahashi et al., Plant Physiol. |
| | | Nicotianamine | | 121 (3), 947-956 (1999) |
| | | aminotransferase from | | 228/379 (60%) |
| | | barley (Hordeum vulgare) | | |
| | | iron siderophore | | |
| | | biosynthetic enzyme gene; | | |
| | | enhances iron uptake by | | |
| | | roots. | | |
| NAS1 | 57-58 | iron acquisition | 0 | Higuchi et al., Plant J. 25 (2), |
| | | Nicotianamine synthase 1 | | 159-167 (2001) |
| | | (NAS1) from rice (Oryza | | 331/332 (99%) |
| | | sativa); iron siderophore | | |
| | | biosynthetic enzyme gene; | | |
| | | enhances iron uptake by | | |
| | | roots. | | |
| NRAMP1 | 59-60 | iron acquisition | 0 | Belouchi et al., Plant Mol. Biol. |
| | | Iron transporter Nramp1 | | 29 (6), 1181-1196 (1995) |
| | | from rice (Oryza sativa); | | 512/518 (98%) |
| | | enhances iron uptake by | | |
| | | roots. | | |
| NRAMP2 | 61-62 | iron acquisition | 0 | Belouchi et al., Plant Mol. Biol. |
| | | Iron transporter from rice | | 33 (6), 1085-1092 (1997) |
| | | (Oryza sativa); enhances | | 459/464 (98%) |
| | | iron uptake by roots. | | |
| PT1 | 63-64 | phosphate acquisition | 0 | Kai and Adachi, unpublished |
| | | Phospate transporter from | | 414/518 (79%) |
| | | tobacco (Nicotiana | | |
| | | tabacum); functions in | | |
| | | uptake of phoshphate, a | | |
| | | key plant nutrient, in roots. | | |

There are a wide variety of methods for analyzing grain quality including, but not limited to, starch analysis, protein content and analysis, phosphate acquisition, and iron acquisition.

A preferred starch content assay is the Sigma Starch Assay Kit (Product no. STA-20; Sigma Chemical Co., St. Louis, Mo.). The protocol is available from the manufacturer.

A preferred protein content assay is the Biorad Bradford Assay Kit (#500-0002; Biorad, Hercules, Calif.).

The phosphate acquisition assay, is essentially performed by growing plants hydroponically under phosphate-limiting conditions (for example, 10 uM final concentration). Leaf phosphorus levels (Murphy and Riley (1962) Anal. Chim. Acta 27: 31-36) and growth (final dry weight and seed weight) are measured, comparing transgenic and non-transgenic plants.

Similarly, for the iron acquisition assay, plants are grown hydroponically under iron-limiting conditions, essentially as described in Thoiron et al. (1997) Plant Cell Env. 20:1051-1060. Assays include measurement of the extent of low iron-induced chlorosis (essentially as described in Takahashi et al. (2001) Nat. Biotech. 19: 466469) and growth measurements (final dry weight and seed weight), comparing transgenic and non-transgenic plants.

NIRS Assay

Near Infrared Reflectance Spectroscopy (NIRS) is used for evaluation of protein and amino acids in seeds by those in the art. Techniques are used to test individual grains or a sample of ground seeds as preferred or required. A number of NIRS assay techniques and devices are available to those in the art for analyzing a variety of samples, including grain quality.

NIRS calibrations for evaluation of a number of feed components (including protein) can be purchased on the open market and the best place to find what is available is probably the manufacturers of NIRS equipment. There are two main suppliers of NIRS-FOSS NIRS systems and Bran & Lubbe. There is also a group run by Pierre Dardenne at the University of Gembloux in Belgium who produce and sell calibrations.

Similarly, Perten Instruments, provides for all sorts of applications for NIR including measuring starch and protein. For altered protein composition, the highest resolution methods available currently are believed to be, for example, high performance capillary electrophoresis as described by George Lookhart in Journal of Chromatography A, 881 (2000) 23-36 and in Electrophoresis 2001, 1503-1509. There is a review in Current Opinion in Biotechnology 1999 on Production of modified polymeric carbohydrates 10,169-174, which is a good source of a range of methods.

The USDA intemet page, also has application notes with a list of methods to analyze grain quality listed under cultivar development, and the ARS News and Information section. And a general reference for the analysis of rice is by Barton et al., Agricultural Research, August 1998, pages 18-21.

The above-disclosed embodiments are illustrative. This disclosure of the invention will place one skilled in the art in possession of many variations of the invention. All such obvious and foreseeable variations are intended to be encompassed by the present invention. All references cited within are hereby incorporated by reference in their entirety.

What is claimed is:

1. An isolated nucleic acid molecule comprising or consisting of a nucleotide sequence, its complement, or its reverse complement, encoding a polypeptide including:
a) a polypeptide sequence listed in even numbered sequences of SEQ ID Nos:2-64, or a fragment, domain, repeat, feature, or chimera thereof;
b) a polypeptide sequence having substantial similarity to (a);
c) a polypeptide sequence encoded by a nucleotide sequence identical to or having substantial similarity to a nucleotide sequence listed in odd numbered sequences of SEQ ID Nos:1-63, or a fragment, domain, or feature thereof, or a sequence complementary thereto;
d) a polypeptide sequence encoded by a nucleotide sequence capable of hybridizing under medium stringency conditions to a nucleotide sequence listed in odd numbered sequences of SEQ ID Nos:1-63, or to a sequence complementary thereto; and
e) a functional fragment of (a), (b), (c) or (d).2. An isolated nucleic acid of claim 1, comprising a nucleotide sequence including:
a) a nucleotide sequence listed in odd numbered sequences of SEQ ID NO:1-63, fragment, domain, or feature thereof;
b) a nucleotide sequence having substantial similarity to (a);
c) a nucleotide sequence capable of hybridizing to (a);
d) a nucleotide sequence complementary to (a), (b) or (c); and
e) a nucleotide sequence which is the reverse complement of (a), (b) or (c).

3. An expression cassette comprising a promoter and the nucleic acid molecule of claim 1.

4. A recombinant vector comprising the expression cassette of claim 3.

5. A cell comprising the expression cassette of claim 3.

6. A transgenic plant comprising the expression cassette of claim 3.

7. Progeny and seed from the transgenic plant of claim 6.

8. The transgenic plant of claim 6, wherein the expression cassette is expressed in the tissue of the epidermis, root, vascular tissue, meristem, cambium, cortex, pith, leaf, and flower.

9. The plant of claim 6, wherein the plant has altered abiotic stress tolerance, enhanced yield, altered disease resistance, or altered nutritional content.

10. The transgenic plant of claim 6, wherein the plant is selected from the group consisting of: rice, wheat, barley, rye, corn, potato, canola, soybean, sunflower, carrot, sweet potato, sugarbeet, bean, pea, chicory, lettuce, cabbage, cauliflower, broccoli, turnip, radish, spinach, asparagus, onion, garlic, eggplant, pepper, celery, squash, pumpkin, cucumber, apple, pear, quince, melon, plum, cherry, peach, nectarine, apricot, strawberry, grape, raspberry, blackberry, pineapple, avocado, papaya, mango, banana, soybean, tobacco, tomato, sorghum and sugarcane.

11. The transgenic plant of claim 10, wherein the plant is rice.

12. The transgenic plant of claim 6, wherein the plant is a monocot.

13. The transgenic plant of claim 12, wherein the monocot is selected from the group consisting ofL maize, wheat, barley, oats, rye, millet, sorghum, trticale, secale, einkorn, spelt, emmer, teff, milo, flax, gramma grass, Tripsacum, and teosinte.

14. A transgenic plant comprising the nucleic acid molecule of claim 2.

15. Progeny and seed from the transgenic plant of claim 14.

16. A method of altering the abiotic stress tolerance of a plant, comprising expressing an expression cassette comprising a nucleic acid molecule encoding a polypeptide having SEQ ID NO:2, 4, 6, 8, or 10 in a plant.

17. A method of altering pathogen or disease resistance in a plant, comprising expressing an expression cassette comprising a nucleic acid molecule encoding a polypeptide having SEQ ID NO:12, 14, 16, 18, 20, 22, or 24 in the plant.

18. A method of altering the grain quality, nutritional composition or yield of a plant, comprising expressing an expression cassette comprising a nucleic acid molecule encoding a polypeptide having SEQ ID NO:26, 28, 30, 32, 34, 36, 38, 40, 42, 44, 46, 48, 50, 52, 54, 56, 58, 60, 62 or 64.

19. The method of claim 18, wherein the altered grairi quality, nutritional composition or yield is altered starch level, altered thiamin level, altered lysine level, altered iron level, altered aleurone enzyme levels, altered vitamin E content, altered protein level, altered time to harvest, altered seed number, altered iron acquisition, or altered phosphate acquisition.

20. A shuffled nucleic acid containing a plurality of nucleotide sequence fragments, wherein at least one of the fragments corresponds to a region of a nucleotide sequence listed in odd numbered sequences of SEQ ID NOS:1-63, and wherein at least two of the plurality of sequence fragments are in an order, from 5′ to 3′ which is not an order in which the plurality of fragments naturally occur in a nucleic acid.

21. A polypeptide comprising:
a) a polypeptide sequence of SEQ ID NO:2;
b) a polypeptide sequence having substantial similarity to (a);
c) a polypeptide sequence encoded by a nucleotide sequence identical or substantially similar to a nucleotide sequence of SEQ ID NO:1;
d) a polypeptide sequence encoded by a nucleic acid molecule capable of hybridizing under high stringency conditions to a nucleic acid molecule listed in SEQ ID NO:1 or to a sequence complementary thereto; and
e) a functional fragment of (a), (b), (c) or (d).

22. A method of producing a polypeptide of claim 21, comprising the steps of:
a) growing recombinant cells comprising an expression cassette under suitable growth conditions, the expression cassette comprising a nucleic acid molecule of claim 1; and
b) isolating the polypeptide from the recombinant cells.

23. A method of decreasing the expression of a nucleic acid molecule of claim 1 in a plant comprising:
(a) expressing in said plant a DNA molecule of claim 1 or a portion thereof in “sense” orientation; or
(b) expressing in said plant a DNA molecule of claim 1 or a portion thereof in “anti-sense” orientation; or
(c) expressing in said plant a ribozyme capable of specifically cleaving a messenger RNA transcript encoded by an endogenous gene corresponding to a DNA molecule of claim 1; or
(d) expressing in a plant an aptamer specifically directed to a protein encoded by a DNA molecules of claim 1; or
(e) expressing in a plant a mutated or a truncated form of a DNA molecule of claim 1;
(f) modifying by homologous recombination in a plant at least one chromosomal copy of the gene corresponding to a DNA molecule of claim 1; or
g) modifying by homologous recombination in a plant at least one chromosomal copy of the regulatory elements of a gene corresponding to any one of the DNA molecules of claim 1; or
h) expressing in said plant a DNA molecule of claim 1 or a portion thereof in the “sense” and “antisense” orientation.

28. An antibody cross-reactive to the polypeptide of claim 21.

29. The polypeptide of claim 21, wherein the polypeptide is involved in a function such as abiotic stress tolerance, enhanced yield, disease resistance or nutritional content.

30. A method of altering the expression of a polypeptide of claim 21 in a plant, comprising the step of expressing an expression cassette of claim 3 in the plant.

31. The method of claim 30, wherein the polypeptide is expressed in a specific location or tissue of a plant.

32. The method of claim 31, wherein the location or tissue is the epidermis, root, vascular tissue, meristem, cambium, cortex, pith, leaf, flower or seed.

33. An isolated product from the plant which comprises an expression cassette comprising a promoter sequence operably linked to an isolated nucleic acid comprising a nucleotide sequence including:
a) a nucleotide sequence listed in odd numbered sequences of SEQ ID NOS:1-63, or fragment, domain, or feature thereof;
b) a nucleotide sequence encoding a polypeptide listed in even numbered sequences of SEQ ID NOS: 2-64;
c) a nucleotide sequence having substantial similarity to (a) or (b);
d) a nucleotide sequence capable of hybridizing to (a) or (b);
e) a nucleotide sequence complementary to (a), (b), (c) or (d); and
f) a nucleotide sequence that is the reverse complement of (a), (b), (c) or (d) according to the present disclosure.

34. The isolated product of claim 33, wherein the isolated product includes an enzyme, a nutritional protein, a structural protein, an amino acid, a lipid, a fatty acid, a polysaccharide, a sugar, an alcohol, an alkaloid, a carotenoid, a propanoid, a steroid, a pigment, a vitamin or a plant hormone.

35. A method of producing a recombinant protein, comprising the steps of:
(a) growing recombinant cells comprising a nucleic acid construct under suitable growth conditions, the construct comprising an expression vector and a nucleic acid including: a nucleic acid encoding a protein as listed in even numbered nucleotide sequences of SEQ ID NOS:2-64, or a nucleic acid sequence listed in odd numbered nucleotide sequences of SEQ ID NOS:1-63, or segments thereof; and
(b) isolating from the recombinant cells the recombinant protein expressed thereby.

36. The method of claim 35, wherein the expression vector includes one or more elements of a promoter-enhancer sequence, a selection marker sequence, an origin of replication, an epitope-tag encoding sequence, an affinity purification-tag encoding sequence, a polyamino acid binding substance, or chitin-binding domain.
